SEQUENCE LISTING (23 sequences)

SEQ ID NO: 1 - protein, 1215 aa, Homo sapiens
MGAMAYPLLLCLLLAQLGLGAVGASRDPQGRPDSPRERTPKGKPHAQQPGRASASDSSAPWSRSTDGTILAQKLAEEVPMDVASYLYTGDSHQLKRANCSGRYELAGLPGKWPALASAHPSLHRALDTLTHATNFLNVMLQSNKSREQNLQDDLDWYQALVWSLLEGEPSISRAAITFSTDSLSAPAPQVFLQATREESRILLQDLSSSAPHLANATLETEWFHGLRRKWRPHLHRRGPNQGPRGLGHSWRRKDGLGGDKSHFKWSPPYLECENGSYKPGWLVTLSSAIYGLQPNLVPEFRGVMKVDINLQKVDIDQCSSDGWFSGTHKCHLNNSECMPIKGLGFVLGAYECICKAGFYHPGVLPVNNFRRRGPDQHISGSTKDVSEEAYVCLPCREGCPFCADDSPCFVQEDKYLRLAIISFQALCMLLDFVSMLVVYHFRKAKSIRASGLILLETILFGSLLLYFPVVILYFEPSTFRCILLRWARLLGFATVYGTVTLKLHRVLKVFLSRTAQRIPYMTGGRVMRMLAVILLVVFWFLIGWTSSVCQNLEKQISLIGQGKTSDHLIFNMCLIDRWDYMTAVAEFLFLLWGVYLCYAVRTVPSAFHEPRYMAVAVHNELIISAIFHTIRFVLASRLQSDWMLMLYFAHTHLTVTVTIGLLLIPKFSHSSNNPRDDIATEAYEDELDMGRSGSYLNSSINSAWSEHSLDPEDIRDELKKLYAQLEIYKRKKMITNNPHLQKKRCSKKGLGRSIMRRITEIPETVSRQCSKEDKEGADHGTAKGTALIRKNPPESSGNTGKSKEETLKNRVFSLKKSHSTYDHVRDQTEESSSLPTESQEEETTENSTLESLSGKKLTQKLKEDSEAESTESVPLVCKSASAHNLSSEKKTGHPRTSMLQKSLSVIASAKEKTLGLAGKTQTAGVEERTKSQKPLPKDKETNRNHSNSDNTETKDPAPQNSNPAEEPRKPQKSGIMKQQRVNPTTANSDLNPGTTQMKDNFDIGEVCPWEVYDLTPGPVPSESKVQKHVSIVASEMEKNPTFSLKEKSHHKPKAAEVCQQSNQKRIDKAEVCLWESQGQSILEDEKLLISKTPVLPERAKEENGGQPRAANVCAGQSEELPPKAVASKTENENLNQIGHQEKKTSSSEENVRGSYNSSNNFQQPLTSRAEVCPWEFETPAQPNAGRSVALPASSALSANKIAGPRKEEIWDSFKV

SEQ ID NO: 2 - protein, 131 aa, Synthetic construct
MAEVQLQESGGGLVQAGGSLRLSCAASGSIGNIYIMGWYRQTPGPQRELVATIRTVRWTKYEDYADSVKGRFTISDDDAKNTVYLQMNSLKPEDTAVYYCNYKDYNAPSDGYWGQGTQVTVSSEPKTPKPQ

SEQ ID NO: 3 - protein, 8 aa, Synthetic construct
GSIGNIYI

SEQ ID NO: 4 - protein, 10 aa, Synthetic construct
IRTVRWTKYE

SEQ ID NO: 5 - protein, 12 aa, Synthetic construct
NYKDYNAPSDGY

SEQ ID NO: 6 - DNA, 54 bp, Synthetic construct
  source 1..54: mol_type = other DNA
gtcgtcggcccagccggccatggccgaggtgcagctggtggagtctgggggagg

SEQ ID NO: 7 - DNA, 54 bp, Synthetic construct
  source 1..54: mol_type = other DNA
gtcgtcggcccagccggccatggccgaggtgcagctgcaggmgtctgggggagg

SEQ ID NO: 8 - DNA, 48 bp, Synthetic construct
  source 1..48: mol_type = other DNA
gtcgtcggcccagccggccatggccgaggtgcagctgcaggcgtctgg

SEQ ID NO: 9 - DNA, 48 bp, Synthetic construct
  source 1..48: mol_type = other DNA
gtcgtcggcccagccggccatggccgaggtgcagctgcaggagtcwgg

SEQ ID NO: 10 - DNA, 35 bp, Synthetic construct
  source 1..35: mol_type = other DNA
gctgctgcggccgcggggtcttcgctgtggtgcgc

SEQ ID NO: 11 - DNA, 35 bp, Synthetic construct
  source 1..35: mol_type = other DNA
gctgctgcggccgcttgtggttttggtgtcttggg

SEQ ID NO: 12 - protein, 417 aa, Homo sapiens
MGAMAYPLLLCLLLAQLGLGAVGASRDPQGRPDSPRERTPKGKPHAQQPGRASASDSSAPWSRSTDGTILAQKLAEEVPMDVASYLYTGDSHQLKRANCSGRYELAGLPGKWPALASAHPSLHRALDTLTHATNFLNVMLQSNKSREQNLQDDLDWYQALVWSLLEGEPSISRAAITFSTDSLSAPAPQVFLQATREESRILLQDLSSSAPHLANATLETEWFHGLRRKWRPHLHRRGPNQGPRGLGHSWRRKDGLGGDKSHFKWSPPYLECENGSYKPGWLVTLSSAIYGLQPNLVPEFRGVMKVDINLQKVDIDQCSSDGWFSGTHKCHLNNSECMPIKGLGFVLGAYECICKAGFYHPGVLPVNNFRRRGPDQHISGSTKDVSEEAYVCLPCREGCPFCADDSPCFVQEDKYLR

SEQ ID NO: 13 - protein, 131 aa, Synthetic construct
MAEVQLQESGGGLVQAGGSLRLSCAASGSGGAGAGMGWYRQTPGPQRELVATIGAVGGAAAGDYADSVKGRFTISDDDAKNTVYLQMNSLKPEDTAVYYCNYKDYNAPSDGYWGQGTQVTVSSEPKTPKPQ

SEQ ID NO: 14 - protein, 8 aa, Synthetic construct
GSGGAGAG

SEQ ID NO: 15 - protein, 10 aa, Synthetic construct
IGAVGGAAAG

SEQ ID NO: 16 - DNA, 38 bp, Synthetic construct
  source 1..38: mol_type = other DNA
gcggcggtgctggcgctggcatgggctggtaccgccag

SEQ ID NO: 17 - DNA, 39 bp, Synthetic construct
  source 1..39: mol_type = other DNA
cgccagcaccgccgcttccagaggctgcacaggagagtc

SEQ ID NO: 18 - DNA, 47 bp, Synthetic construct
  source 1..47: mol_type = other DNA
cagccgcagccgcagccgcagccgactatgcagactccgtaaagggc

SEQ ID NO: 19 - DNA, 40 bp, Synthetic construct
  source 1..40: mol_type = other DNA
ctgcggctgcggctgcggctgcagttgcgaccagctcgcg

SEQ ID NO: 20 - protein, 6 aa, Synthetic construct
IGNIYI

SEQ ID NO: 21 - protein, 6 aa, Synthetic construct
GGAGAG

SEQ ID NO: 22 - protein, 9 aa, Synthetic construct
RTVRWTKYE

SEQ ID NO: 23 - protein, 9 aa, Synthetic construct
GAVGGAAAG

(19) United States
(12) Patent Application Publication
(10) Pub. No.: US 2026/0193347 A1
(43) Pub. Date: Jul. 9, 2026

(54) TARGETING GPR158 (MGLYR) WITH NANOBODIES FOR THERAPEUTIC BENEFITS

(71) Applicant: UNIVERSITY OF FLORIDA RESEARCH FOUNDATION, INCORPORATED, Gainesville, FL (US)

(72) Inventors: Kirill MARTEMYANOV, Jupiter, FL (US); Thibaut LABOUTE, Jensen Beach, FL (US)

(21) Appl. No.: 19/133,585

(22) PCT Filed: Nov. 28, 2023
(86) PCT No.: PCT/US2023/081400

§ 371 Date: May 28, 2025

Related U.S. Application Data
(60) Provisional application No. 63/385,387, filed on Nov. 29, 2022.
(60) Provisional application No. 63/503,281, filed on May 19, 2023.

(51) Int. Cl. C07K 16/28; A61K 39/00; A61P 25/24; G01N 33/74

(52) U.S. Cl. CPC C07K 16/28; A61P 25/24; G01N 33/74; A61K 2039/505; C07K 2317/22; C07K 2317/569; C07K 2317/76; C07K 2317/92; G01N 2333/726

(57) ABSTRACT

The invention provides antibodies that specifically bind GPR158 and inhibit GAP activity of GPR158 via RGS7/Gβ5. The antibodies are useful in the diagnosis and treatment of affective disorders, mood disorders, and brain disorders.

### CROSS REFERENCE TO RELATED APPLICATIONS

This application is related to U.S. Provisional Application No. 63/385,387, filed Nov. 29, 2022, and U.S. Provisional Application No. 63/503,281, filed May 19, 2023, each of which is incorporated by reference in its entirety for all purposes.

### STATEMENT REGARDING FEDERALLY SPONSORED RESEARCH OR DEVELOPMENT

This invention was made with government support under Grant No. R01 MH105482 awarded by the National Institutes of Health. The government has certain rights in the invention.

### REFERENCE TO A SEQUENCE LISTING

The Sequence Listing written in file 605274SEQLST.xml is 22.5 kilobytes, was created on Nov. 22, 2023, and is hereby incorporated by reference.

### BACKGROUND

Glycine is the simplest amino acid ubiquitously present in all mammalian tissues. Glycine serves as an inhibitory neurotransmitter but it can be excitatory in developing neurons (1,2). Glycine is transported into the axonal terminals and packaged into vesicles for discrete synaptic release by system of dedicated glycine transporters (3). Glycinergic neurons are distributed across the brain, however glycine can also be released by glial cells (4). Known receptors for glycine belong to family of pentameric ligand-gated ion channels (5). Glycine also serves as co-agonist of N-methyl-D-aspartate (NMDA) receptors (6). Interestingly, metabotropic neuromodulatory effects of glycine have been observed (7,8) but no receptors mediating these actions have been found. Glycine has distinct effects on neural circuits (4) and glycinergic transmission has been implicated in pathological conditions including depression (9-11). A chemically related compound to glycine, taurine engages many of the same receptor targets to modulate neuronal signaling leading to a debate that it could serve as a neuromodulator (12).

Metabotropic neuromodulation in the nervous system is mainly mediated by the G Protein Coupled Receptors (GPCR). GPCRs play essential roles in a neuronal physiology, pathology and present an unrivaled targets for the development of drugs (13). Canonically, GPCRs transduce their signals by activating heterotrimeric G proteins (14-16). However, G protein independent modes of signal transduction triggered by the recruitment of β-arrestins and other scaffolds to activated GPCRs have also been described (17-19). G protein signaling is tightly regulated by the Regulator of Protein Signaling (RGS) proteins which facilitate their deactivation (20). Recent evidence points to significant interactions of RGS proteins with several different GPCRs prominently including members of the Class C receptors (21-25).

With the exception of glycine and taurine, GPCRs are known to mediate the effects of all major neurotransmitters. However, many GPCRs still remain orphan with no identified endogenous ligands. It is generally agreed that orphan GPCRs have high potential for obtaining novel insights into physiology and for drug development (26, 27).

GPR158 is one of the most abundant orphan GPCRs in the brain. It has an unusual biology coupling to RGS proteins to transduce signals. In neurons, it regulates signaling to second messenger cAMP and controls key ion channels, kinases and neurotrophic factors involved in neuronal excitability and synaptic transmission (28). Accordingly, GPR158 has been heavily implicated in cognition and affective states (29-31). Genetic suppression of GPR158 in mice results in prominent antidepressant phenotype and stress resiliency making GPR158 an attractive target for development of novel anti-depressants (29).

Major depressive disorder (MDD) is a prevalent neuropsychiatric condition that affects nearly 5% of the population in developed countries (34). While there has been tremendous progress in treating the depression, the efficacy of currently approved therapeutics is limited (35). Exploring new strategies and drug targets has been deemed a relevant goal for managing MDD (36-38).

The molecular etiology of MDD is complex and not fully understood. Disbalance in neurotransmitter signaling is thought to trigger a range of maladaptive changes involving ion channels, kinases and second messengers, in particular cAMP, influencing synaptic communication and neuronal excitability (39-40). These changes are precipitated by environmental factors such as stress, a major aggravating factor in MDD (41). The neuronal circuits underlying processing of emotional states and impacted by MDD are similarly complex and involve many structures including the prefrontal cortex (PFC), a region heavily implicated in affective disorders and the effects of stress (42).

MDD treatment has traditionally focused on the elements that mediate signaling by monoamine neurotransmitters targeted by a vast majority of currently approved antidepressants (43). However, several recent medications also target GABA, glutamate and opioid receptors highlighting the potential of other neurotransmitter systems in developing MDD treatments (44-45). One such untapped system involves the neurotransmitter glycine. It is released by specific neurons and has unique effects on neural circuits and the activity of neurons (1, 46). Glycine and its related naturally occurring compound taurine have been heavily implicated in mood regulation and depression (9, 11, 47).

The effects of glycine had been thought to be mediated mainly by dedicated glycine receptor GlyR, an inhibitory ion channel (5). Recently, a metabotropic receptor for glycine was discovered—mGlyR (48). Formerly known as an orphan receptor GPR158, it exerts excitatory effects on neurons via modulation of second messenger cAMP. The mGlyR is prominently expressed in the PFC and its expression is regulated by stress (29, 49). The levels of mGlyR are markedly upregulated in patients diagnosed with MDD and its knockout in mice produces antidepressant phenotype and stress resilience (29). The mGlyR employs extracellular Cache domain for glycine recognition (48). Binding of glycine or taurine to the ligand binding pocket in this domain changes the activity of the associated Regulator of G Protein Signaling (RGS) complex at the intracellular side. This influences G protein signaling to second messengers and ion channels thereby transducing the signal (48). Notably, loss of RGS regulation also produces antidepressant effects in mouse models (50). Together, these observations point to mGlyR as an attractive target for developing of new anti-depressant therapies.

### SUMMARY OF THE INVENTION

In one aspect, the invention provides an isolated monoclonal antibody that competes for binding to human GPR158 with antibody Nb20. Some antibodies bind to the same epitope on human GPR158 as antibody Nb20.

Some antibodies comprise three heavy chain CDRs of antibody Nb20, wherein Nb20 is a llama antibody characterized by a heavy chain variable region having an amino acid sequence comprising SEQ ID NO:2. In some antibodies, the three heavy chain CDRs are as defined by IMGT (SEQ ID NOs: 3-5). In some antibodies, the heavy chain variable region comprises an amino acid sequence of SEQ ID NO:2.

Some antibodies are Nb20 or a chimeric, veneered, or humanized form thereof. For example, the antibody can be a humanized antibody. Some antibodies are a humanized Nb20 antibody that specifically binds to human GPR158, wherein Nb20 is a llama antibody characterized by a mature heavy chain variable region of SEQ ID NO:2. Some antibodies comprise a humanized mature heavy chain variable region comprising the three heavy chain CDRs of Nb20. In some antibodies, the CDRs are of a definition selected from the group of Kabat, Chothia, Kabat/Chothia Composite, AbM, Contact, and IMGT.

The antibody can be an intact antibody. The antibody can be a binding fragment. In some such antibodies the binding fragment is a single-chain antibody, Fab, or F(ab′)₂ fragment. The antibody can be a Fab fragment, or single chain Fv. The antibody can be a nanobody.

In some antibodies, the isotype is human IgG1. In some antibodies, the mature heavy chain variable region is fused to a heavy chain constant region. In some antibodies, the heavy chain constant region is a mutant form of a natural human heavy chain constant region which has reduced binding to a Fcγ receptor relative to the natural human heavy chain constant region. In some antibodies, the heavy chain constant region is of IgG1 isotype.

Some antibodies have at least one mutation in the constant region. In some antibodies, the mutation reduces complement fixation or activation by the constant region. Some antibodies have a mutation at one or more of positions 241, 264, 265, 270, 296, 297, 318, 320, 322, 329 and 331 by EU numbering. Some antibodies have alanine at positions 318, 320 and 322.

Some antibodies have a human IgG2, IgG3, or IgG4 isotype. Some antibodies are at least 95% w/w pure. Some antibodies are conjugated to a therapeutic, cytotoxic, cytostatic, neurotrophic, or neuroprotective agent.

In another aspect, the invention provides a pharmaceutical composition comprising any of the antibodies disclosed herein and a pharmaceutically-acceptable carrier.

In another aspect, the invention provides a nucleic acid encoding the heavy chain of any of the antibodies disclosed herein. In another aspect, the invention provides a recombinant expression vector comprising such a nucleic acid. In another aspect, the invention provides a host cell transformed such a recombinant expression vector.

In another aspect, the invention provides a method of humanizing a nanobody, the method comprising: (a) selecting one or more acceptor humanized nanobody scaffolds; (b) identifying amino acid residues of the nanobody to be retained; (c) synthesizing a nucleic acid encoding a humanized heavy chain comprising CDRs of the nanobody heavy chain; and (d) expressing the nucleic acids in a host cell to produce a humanized nanobody; wherein the nanobody is Nb20, wherein Nb20 is characterized by a mature heavy chain variable region of SEQ ID NO:2.

In another aspect, the invention provides a method of producing a humanized, chimeric, or veneered antibody, the method comprising: (a) culturing cells transformed with a nucleic acid encoding the heavy chain of the antibody, so that the cells secrete the antibody; and (b) purifying the antibody from cell culture media; wherein the antibody is a humanized, chimeric, or veneered form of an antibody characterized by a mature heavy chain variable region of SEQ ID NO: 2.

In another aspect, the invention provides a method of producing a cell line producing a humanized, chimeric, or veneered antibody, the method comprising: (a) introducing a vector encoding a heavy chain of an antibody and a selectable marker into cells; (b) propagating the cells under conditions to select for cells having increased copy number of the vector; (c) isolating single cells from the selected cells; and (d) banking cells cloned from a single cell selected based on yield of antibody; wherein the antibody is a humanized, chimeric, or veneered form of an antibody characterized by a mature heavy chain variable region of SEQ ID NO:2. Some such methods further comprise propagating the cells under selective conditions and screening for cell lines naturally expressing and secreting at least 100 mg/L/10⁶ cells/24 h.

In another aspect, the invention provides a method of treating or effecting prophylaxis of an affective disorder, a mood disorder, or a brain disorder in a subject, comprising administering to the subject an effective regime of any of the antibodies disclosed herein and thereby treating or effecting prophylaxis of the affective disorder, mood disorder, or brain disorder in the subject. In some such methods, the affective disorder, mood disorder, or brain disorder is depression, disruptive mood dysregulation disorder, major depressive disorder (MDD), dysthymia, stress induced depression, a generalized mood disorder, chronic stress disorder, anhedonia, or an anxiety disorder.

In another aspect, the invention provides a method of detecting of GPR158 in a biological sample from a subject, comprising contacting the biological sample with an effective amount of any of the antibodies disclosed herein. Some such methods further comprise detecting the binding of antibody to GPR158. Some such methods further comprise comparing binding of the antibody to the biological sample with binding of the antibody to a control sample.

A further understanding of the nature and advantages of the present invention may be realized by reference to the remaining portions of the specification and claims.

### BRIEF DESCRIPTION OF THE DRAWINGS

FIG. 1 depicts a strategy for the identification of nanobodies specifically interacting with GPR158. Briefly, a llama was immunized with membranes expressing GPR158 and lymphocytes were isolated from llama's blood. mRNA was extracted from lymphocytes and reverse transcription was performed on the extracted mRNA. Reverse transcribed cDNA were amplified using primers made to amplify coding sequence for the variable domain of IgG2 and IgG3 (no light chain). A phage library was constructed with amplified cDNA, followed by 3 rounds of panning on mGlyR to enrich for specific binders which were isolated and tested.

FIG. 2 depicts sequence of Nanobody-20 (Nb20) (SEQ ID NO:2).

FIGS. 3A-3B show Nanobody-20 specifically interacts with GPR158 (mGlyR). FIG. 3A, top left panel, is a schematic of recombinant Nb20 protein binding to full-length GPR158 expressed in HEK293 cells transiently transfected. FIG. 3A, top right panel, depicts results of flow cytometry study of purified recombinant Nb20 protein binding to full-length GPR158 expressed in HEK293 cells. x-axis measures Nb20 binding, and γ-axis measures expression of GPR158 in cells. FIG. 3A, bottom left panel, depicts histograms of binding by flow cytometry of cells transiently expressing GPR158 or mock transfected or dopamine DIR transfected cells incubated with Nb20. Only cells expressing GPR158 show a rightward shift of the histogram demonstrating specificity of the Nb20. FIG. 3A, bottom right panel, depicts dose response profiles of Nb20 binding experiment to cells expressing GPR158 in flow cytometry experiments and shows specificity of Nb20 binding only cells expressing GPR158. FIG. 3B, left panel, is a schematic of SPR strategy showing binding of Nb20 to purified extracellular domain of GPR158 (ectodomain). FIG. 3B, right panel, depicts SPR sensograms of binding and dissociation of Nb20.

FIGS. 4A-4B show Nanobody-20 inhibits GPR158 (mGlyR) signalling via regulating GAP activity of RGS7. FIG. 4A depicts schematics of the BRET-based GAP assay to study activity of mGlyR. First, Gao is activated by dopamine D2R receptor. After reaching steady state, D2R antagonist haloperidol is injected and kinetics of G protein deactivation is monitored by following quenching of the BRET signal. FIG. 4B, left panel, depicts traces of BRET signal showing Gao activation and deactivation time course with or without Nb20 treatment in cells transfected with GPR158. Nb20 significantly decelerated Gao deactivation through GPR158-RGS7/Gβ5 complex. FIG. 4B, right panel, depicts quantification of deactivation time constant (1/τ of the reactions presented in right panels with different transfection conditions. 1/τ is calculated from deactivation curves. Effect of the Nb20 was only measured when both GPR158 and RGS7/Gβ5 are co-transfected. Solid circles (BRET B), circles with no fill (Nb), columns with angled-line fill (BRET B), columns with no fill (Nb).

FIGS. 5A-5G show development of mGlyR-selective nanobodies. A) Schematic of the nanobody development pipeline. A phage library was constructed from leukocytes of a llama immunized with mGlyR, followed by 3 rounds of panning on mGlyR to enrich for specific binders which were isolated and tested. B) Schematic of the detection strategy in flow cytometry experiments. C) Analysis of nanobody binding by flow cytometry of HEK cells transiently expressing mGlyR incubated with or without Nb20 and anti-myc-APC conjugated antibody. Percentages of cells in each quadrant are indicated. D) Dose-response profiles of representative Nb20 binding experiment to cells expressing mGlyR in flow cytometry experiments. Concentrations of Nb20 are shown. E) Quantification of data in 5D. Error bars are SEM values (n=3 independent experiments). F) Schematic of the surface plasmon resonance (SPR) assays that detect Nb20 binding to the chip containing immobilized recombinant ectodomain of mGlyR (Ecto-mGlyR). G) SPR sensogram of binding and dissociation of Nb20.

FIGS. 6A-6B show specificity of Nb20 for mGlyR. A) Schematic of the detection strategy and analysis of the binding. Anti-Myc APC conjugated antibody±Nb20 were incubated on cells not expressing mGlyR. Percentages of cells in each quadrant are indicated. B) Analysis of nanobody binding by flow cytometry of HEK suspension cells transiently expressing mGlyR or other receptors incubated with Nb20 and anti-myc-APC conjugated antibody.

FIG. 7 shows the effect of glycine on Nb20 binding to mGlyR. HEK293 cells transfected with mGlyR were incubated with Nb20 (1 μM) and anti-myc APC conjugated antibody in the presence or absence of 100 μM of glycine.

FIGS. 8A-8E show regulation of GAP activity of mGlyR-RGS7/Gβ5 complex by Nb20. A) Schematics of the BRET-based GAP assay to study activity of mGlyR. First, Gao is activated by dopamine D2R receptor. After reaching steady state, D2R antagonist haloperidol is injected and kinetics of G protein deactivation is monitored by following quenching of the BRET signal. B, C) Traces of BRET signal showing Gao activation and deactivation time course with or without Nb20 treatment in cells without mGlyR (B) or cells transfected with mGlyR (C). D) Quantification of deactivation time constant of the reactions presented in 8B and 8C. 1/τ is calculated from deactivation curves of n=3 independent experiments conducted in triplicate from each cell transfection group. Data represent mean±SEM. ****p<0.0001, ns (not significant)=p>0.05, two-way ANOVA. Columns with angled-line fill (Mock), columns with no fill (Nb20) E) Dose-response profile of changes in GAP activity (KGAP) calculated by subtracting the baseline deactivation rate (1/τ) from the rate of the reaction in the presence of GPR158-RGS7-Gβ5. Data are mean±SEM of n=3 independent experiments conducted in triplicate.

FIGS. 9A-9B show regulation of mGlyR activity by Nb20 and glycine. A) Representative traces of BRET signal showing Goo activation and deactivation time course with or without Nb20 or glycine or both in HEK293 cells transfected with mGlyR and D2R. D2R was activated by dopamine at time point 0 s. Arrow indicate addition of D2R antagonist haloperidol. B) Quantification of deactivation time constant of the reactions presented in 9A. 1/τ is calculated from deactivation curves of n=3 independent experiments conducted in triplicate from each cell transfection group. Data represent mean±SEM ****p<0.0001, one-way ANOVA. No statistically significant differences (p>0.05) were detected between Nb20, glycine or both treatment groups were observed.

FIGS. 10A-10E show antidepressant effects of Nb20 administration in mice. A-C) Vehicle or nanobody (9.6 μg) in naïve mice. A) Schematic of the intracerebroventricular (ICV) injection strategy for the administration of vehicle or Nb20 to mice. B) Evaluation of mice injected with Nb20 or vehicle control in a panel of behavioral tests consisting of elevated plus maze (EPM), marble burying (MB), tail suspension test (TST), and forced swim test (TST) (n=12 mice in vehicle (6 males and 6 females) group and 11 (6 males and 5 females) in ketamine treated and 12 (6 males and 6 females) in Nb20 treated). C) Calculation of emotionality scores based on superscoring of four behavioral tests. D) Schematic of the intranasal delivery for the administration of ketamine, Nb20 or control Nb20* to mice, timeline and experimental strategy for stress induction, treatment and behavioral evaluation. E) Evaluation of mice in behavioral paradigms as indicated (n=12 mice/group: 6 males and 6 females). Data are mean±SEM (Nonparametric One-way ANOVA; Dunnett's test, *p<0.05, **p<0.01, ***p<0.001, ****p<0.0001). Columns with angled-line fill (Nb20*), columns with no fill (Nb20), columns with cross-hatch fill (Ketamine).

FIG. 11 shows anti-depressant effects of Nb20 persist for two weeks after administration in mice. Mice were evaluated in elevated plus maze (EPM), marble burying (MB), tail suspension test (TST), and forced swim test (TST) paradigms 2 weeks after ICV injection of Nb20 or vehicle control. (n=8 mice in vehicle (4 males and 4 females) and 7 (4 males and 3 females) in Nb20 treated groups). Calculation of emotionality scores based on superscoring of four behavioral tests. Data are mean±SEM (Nonparametric One-way ANOVA; Kruskal-Wallis test, *p<0.05, **p<0.01, ***p<0.001, ****p<0.0001). Columns with angled-line fill (Vehicle), columns with no fill (Nb20).

FIG. 12 shows effect of the control nanobody (Nb20*) in mice. Mutated Nb20* (9.6 μg) incapable of binding to mGlyR or vehicle were injected ICV in naïve mice. Mice were evaluated in elevated plus maze (EPM), marble burying (MB), tail suspension test (TST), and forced swim test (TST) paradigms (n=18 mice in vehicle (9 males and 9 females) and 36 (18 males and 18 females) in Nb20* treated groups). Data are mean±SEM (Nonparametric One-way ANOVA; Kruskal-Wallis test, ns=p>0.05). Columns with angled-line fill (Vehicle), columns with no fill (Nb20*).

FIG. 13 shows effects of the initial treatment of mice with Nb20 or ketamine. After undergoing CVS mice were intranasally treated with ketamine (20 mg/mg), Nb20 (9.6 μg) or control Nb20* (9.6 μg) and evaluated 24 h after treatment in marble burying (MB), tail suspension test (TST), and forced swim test (TST) paradigms (n=10 mice/group: 5 males and 5 females). Data are mean±SEM (Nonparametric One-way ANOVA; Kruskal-Wallis test, ns=p>0.05, *p<0.05.) Columns with angled-line fill (Nb20*), columns with no fill (Nb20), columns with cross-hatch fill (Ketamine).

FIG. 14 shows quantification of BDNF in mice treated by ketamine or Nb20. Prefrontal cortex tissue punches (2 mm) from mice treated twice with Nb20*, Nb20 or ketamine were analyzed for the presence of BDNF by ELISA (n=10 mice/group: 5 males and 5 females). Data are mean±SEM (Nonparametric One-way ANOVA; Kruskal-Wallis test, **p<0.01, **p<0.001). Column with angled-line fill (Nb20*), column with no fill (Nb20), column with cross-hatch fill (Ketamine).

FIGS. 15A-15E show effect of Nb20 on neuronal excitability. A) Schematic of the electrophysiological recordings in slice preparation targeting mPFC neurons of layer II-III in WT mice. Slices were incubated with ACSF (Ctrl), Nb20 or mutated Nb20* before recording B) Representative traces of voltage responses to a 200 pA current ramp injection under different conditions. C) Quantification of changes in excitability by number of action potentials fired in response to 200 pA current ramp (n=6-8 neurons, from 3-6 mice). Nonparametric One-way ANOVA; Kruskal-Wallis test, **p<0.01, *p<0.05 and ns=p>0.05. D) Quantification of changes in excitability by rheobase current (n=6-8 neurons, from 3-6 mice). Nonparametric One-way ANOVA; Kruskal-Wallis test, **p<0.01, *p<0.05 and ns=p>0.05. E) Resting membrane potential of layer II-III pyramidal neurons in WT mice (n=6-8 neurons, from 3-6 mice). One-way ANOVA; Kruskal-Wallis test, ns=p>0.05. In 15C, 15D, and 15E, Columns with angled-line fill (Ctrl), columns with no fill (Nb20), columns with cross-hatch fill (Nb20*).

FIG. 16 depicts evaluation of stress paradigm in vehicle injected mice. Evaluation of stressed vs non-stressed mice injected with vehicle control in a panel of behavioral tests consisting of marble burying (MB), splash test (ST), tail suspension test (TST), and forced swim test (TST) (n=12 mice in both groups (6 males and 6 females). Calculation of emotionality scores based on superscoring of four behavioral tests. Data are mean±SEM (Unpaired t-test, *p<0.05, **p<0.01, ***p<0.001, ****p<0.0001).

FIGS. 17A-17G depict CryoEM structure of the mGlyR in complex with Nb20. A) Side view of the cryo-EM map (left) and corresponding model (right) of the mGlyR-Nb20 homodimer, with individual monomers shown. Phospholipids and cholesterols are shown. B) Cryo-EM map (left) and corresponding model (right) of the Nb20-mGlyR-RGS7-Gβ5 homodimer, with monomers as in (A). RGS7 and Gβ5 are marked. C) Top view of Nb20-bound Cache domain of mGlyR. D) Detailed interaction network between Nb20 and the Cache domain. (E) Side view of the TM-based structural superimposition of mGlyR-apo structure with that of mGlyR-Nb20 structure. F) A 90° rotation relative to 17E showing a top view of the structural comparison of the Cache domain. G) Structural superimposition of RGS bound to mGlyR-apo and Nb20-mGlyR.

FIG. 18 depicts Work flow of cryo-EM data processing. Cryo-EM processing steps to obtain high resolution maps of human mGlyR-Nb20 and Nb20-mGlyR-RGS7-Gβ5 complexes.

FIGS. 19A-19F depicts Cryo-EM data processing of human mGlyR-Nb20 and Nb20-mGlyR-RGS7-Gβ5 complexes. A) Representative cryo-EM micrograph, the scale bar corresponds to 100 nm. B) Representative 2D class averages for mGlyR-Nb20 and Nb20-mGlyR-RGS7-Gβ5 complex. C) and D) Gold standard Fourier shell correlation (FSC) curves (Resolutions reported for the maps determined by an FSC cut-off value of 0.143 (horizontal line marked by arrow) for mGlyR-Nb20 (3.47 Å) (D) Nb20-mGlyR-RGS7-Gβ5 complex (3.89 Å). E) and F) Euler angle orientation distribution plots, from CryoSPARC, for the final maps of mGlyR-Nb20 (D) and Nb20-mGlyR-RGS7-Gβ5 complexes.

FIGS. 20a-20B depict validation of Nb20 binding determinants by mutagenesis. A) The binding ability of Nb20* on mGlyR was monitored by flow cytometry. 1.10⁶ cells transiently transfected to express mGlyR were incubated with 1 μM of Nb20 or Nb20* or vehicle and anti-Myc APC conjugated antibody and binding was measured. A) representative histogram of the binding measurement. B) Quantification of anti-Myc-APC binding detected in flow cytometry experiments. Data represent mean±SEM of n=3 independent experiments, ns=p>0.05, ****p<0.0001, one-way ANOVA.

FIGS. 21A-21G depict Cryo-EM structures of Nb20-bound mGlyR. A) Top view of the cryo-EM map showing Nb20 bound to mGlyR. B) Putative ligand binding pocket view highlighting residues involved in ligand binding. C) Side view and 90° rotated view of the global structural superimposition of mGlyR-Nb20 and mGlyR-apo. E) Comparison of the stalk domain between mGlyR-Nb20 and mGlyR-apo structures with global alignment. E) Structural changes in 7TM region of mGlyR-apo compared to Nb20-mGlyR as viewed from the extracellular side. F) TM-anchored structural superimposition of mGlyR-RGS7/Gβ5 with Nb20-mGlyR-RGS7/Gβ5. G) Bottom view of Gβ5 from the alignment in 21F.

FIG. 22 presents other exemplary FRET and BRET biosensors useful in screening assays for antibodies of the invention are shown in FIG. 22 (see Kim, H. et al, (2022) Front. Cell Dev. Biol. 10:1007893). Column 1 shows detection step, column 2 shows target GPCR, column 3 shows ligand used, column 4 shows detection method, column 5 shows FRET or BRET pair, column 6 shows cell lines used, column 7 shows notes, and column 8 shows reference.

GitI, Glutamate/aspartate import solute-binding protein; α2AAR, α2A adrenergic receptor; β1AR, β1 adrenergic receptor; β2AR, β2 adrenergic receptor; A2AR, A2A adenosine receptors; B1R, β1-bradykinin receptor; β2R, β2-bradykinin receptor; H1R, Histamine H1 receptor; mAChR, Muscarinic acetylcholine receptor; PTH1R, Parathyroid hormone 1 receptor; AT1R, Angiotensin II type 1 receptor; V1AR, Vasopressin receptor 1A; A1AR, ALA adenosine receptors; GLP-1R, Glucagon like peptide-1 receptor; V2R, Vasopressin receptor 2; SHT2A, Serotonin receptor 2A; DRD2, Dopamine receptor 2; mGluR1, Metabotropic glutamate receptor 1; PTH, Parathyroid hormone; AVP, Arginine Vasopressin; GLP-1, Glucagon like peptide 1; DOI, 2,5-Dimethoxy-4-iodoamphetamine.
BERKY: BRET-based biosensor with ER/K linker and YFP;
Mini-G: Engineered G alpha protein that contains only essential sequences for the coupling to the GPCR

### BRIEF DESCRIPTION OF THE SEQUENCES

SEQ ID NO: 1 sets forth the amino acid sequence of human GPR158.

SEQ ID NO:2 sets forth the amino acid sequence of llama nanobody Nb20.

SEQ ID NO:3 sets forth the amino acid sequence of llama nanobody Nb20 IMGT CDR-H1.

SEQ ID NO:4 sets forth the amino acid sequence of llama nanobody Nb20 IMGT CDR-H2.

SEQ ID NO:5 sets forth the amino acid sequence of llama nanobody Nb20 IMGT CDR-H3.

SEQ ID NO:6 sets forth the nucleotide sequence of forward primer VH_11.

SEQ ID NO:7 sets forth the nucleotide sequence of forward primer VH_12.

SEQ ID NO:8 sets forth the nucleotide sequence of forward primer VH_14.

SEQ ID NO:9 sets forth the nucleotide sequence of forward primer VH_13.

SEQ ID NO: 10 sets forth the nucleotide sequence of reverse primer VH_sh.

SEQ ID NO:11 sets forth the nucleotide sequence of reverse primer VH_lg.

SEQ ID NO:12 sets forth the amino acid sequence of GPR158 ectodomain.

SEQ ID NO: 13 sets forth the amino acid sequence of Nb20* nanobody.

SEQ ID NO: 14 sets forth the amino acid sequence of Nb*20 IMGT CDR-H1.

SEQ ID NO: 15 sets forth the amino acid sequence of Nb*20 IMGT CDR-H2.

SEQ ID NO:16 sets forth the nucleotide sequence of CDR1 mutation forward primer.

SEQ ID NO:17 sets forth the nucleotide sequence of CDR1 mutation reverse primer.

SEQ ID NO:18 sets forth the nucleotide sequence of CDR2 mutation forward primer.

SEQ ID NO:19 sets forth the nucleotide sequence of CDR2 mutation reverse primer.

SEQ ID NO:20 sets forth the amino acid sequence of residues 30 through 35 of Nb20 nanobody SEQ ID NO:2.

SEQ ID NO:21 sets forth the amino acid sequence of residues 30 through 35 of Nb20* sequence SEQ ID NO:13.

SEQ ID NO:22 sets forth the amino acid sequence of residues 54 through 62 of Nb20 nanobody SEQ ID NO:2.

SEQ ID NO:23 sets forth the amino acid sequence of residues 54 through 62 of Nb20* sequence SEQ ID NO:13.

### Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. For example, the term “an antibody” or “at least one antibody” can include a plurality of antibodies, including mixtures thereof. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. Also, the use of “comprise,” “comprises,” “comprising.” “contain,” “contains,” “containing,” “include,” “includes,” and “including” are not intended to be limiting. Unless otherwise apparent from the context, the term “about” encompasses insubstantial variations, such as values within a standard margin of error of measurement (e.g., SEM) of a stated value. Statistical significance means p≤0.05.

It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s), or method step(s) that do not materially change the basic and novel characteristics of the compositions and methods described herein may be included in those compositions or methods.

Designation of a range of values includes all integers within or defining the range, and all subranges defined by integers within the range. All ranges are to be interpreted as encompassing the endpoints in the absence of express exclusions, such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with disease or condition or those in which disease or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom. The terms “treatment,” “therapeutic method,” and their cognates refer to treatment and prophylactic/preventative measures. Those in need of treatment may include individuals already having a particular medical disorder as well as those who may ultimately acquire the disorder. The need for treatment is assessed, for example, by the presence of one or more risk factors associated with the development of a disorder, the presence or progression of a disorder, or likely receptiveness to treatment of a subject having the disorder. Treatment may include slowing or reversing the progression of a disorder.

Examples of a disease, disorder, or condition associated with an affective disorder, a mood disorder, or a brain disorder are depression, disruptive mood dysregulation disorder, major depressive disorder (MDD), dysthymia, stress induced depression, a generalized mood disorder, chronic stress disorder, anhedonia, and an anxiety disorder. Examples of an affective disorder, a mood disorder or a brain disorder are depression, disruptive mood dysregulation disorder, major depressive disorder (MDD), dysthymia, stress induced depression, a generalized mood disorder, chronic stress disorder, anhedonia, and an anxiety disorder. A “GPR158-related disease” includes an affective disorder, a mood disorder, or a brain disorder and an affective disorder, a mood disorder, or a brain disorder associated with depression, disruptive mood dysregulation disorder, major depressive disorder (MDD), dysthymia, stress induced depression, a generalized mood disorder, chronic stress disorder, anhedonia, and an anxiety disorder. Examples of “GPR158-related disease” are an affective disorder, a mood disorder, a brain disorder, depression, disruptive mood dysregulation disorder, major depressive disorder (MDD), dysthymia, stress induced depression, a generalized mood disorder, chronic stress disorder, anhedonia, and an anxiety disorder.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “individual” or “subject” refers to any mammal, including any animal classified as such, including humans, non-human primates, primates, baboons, chimpanzees, monkeys, cynomolgus, marmoset, rhesus, llamas, alpacas, camels, rodents (e.g., mice, rats), rabbits, cats, dogs, horses, cows, sheep, goats, pigs, ferrets, guinea pigs, hamsters, gerbils etc.

“Orthologs” are genes and products thereof in different species that evolved from a common ancestral gene by speciation and retain the same or similar function. An ortholog is a gene that is related by vertical descent and is responsible for substantially the same or identical functions in different organisms. For example, mouse GPR158 and human GPR158 can be considered orthologs. Genes may share sequence similarity of sufficient amount to indicate they are orthologs. Protein may share three-dimensional structure of sufficient amount to indicate the proteins and the genes encoding them are orthologs. Methods of identifying orthologs are known in the art.

Monoclonal antibodies or other biological entities are typically provided in isolated form. This means that an antibody or other biologically entity is typically at least 50% w/w pure of interfering proteins and other contaminants arising from its production or purification but does not exclude the possibility that the monoclonal antibody is combined with an excess of pharmaceutically acceptable carrier(s) or other vehicle intended to facilitate its use. Sometimes monoclonal antibodies are at least 60%, 70%, 80%, 90%, 95% or 99% w/w pure of interfering proteins and contaminants from production or purification. Often an isolated monoclonal antibody or other biological entity is the predominant macromolecular species remaining after its purification.

Specific binding of an antibody to its target antigen means an affinity and/or avidity of at least 10⁶, 10⁷, 10^{8,} 10⁹, 10¹⁰, 10¹¹, or 10¹² M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. See generally, Fundamental Immunology, Paul, W., ed., 2nd ed. Raven Press, N.Y., 1989, Ch. 7 (incorporated by reference in its entirety for all purposes).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3. When the application discloses a VL sequence with R as the C-terminal residue, the R can alternatively be considered as being the N-terminal residue of the light chain constant region. Thus, the application should also be understood as disclosing the VL sequence without the C-terminal R.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al (bioinfo.org.uk/abs), and IMGT definition (imgt.org/IMGTScientificChart/Numbering/IMGTnumberingCDR_VK.html; also see Ehrenmann F., Kaas Q. and Lefranc M.-P. Nucleic Acids Res., 38:D301-D307 (2010) and Ehrenmann F., Kaas Q. and Lefranc M.-P. Nucleic Acids Res., 38:D301-D307 (2010)) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

TABLE 1
Conventional Definitions of CDRs Using Kabat Numbering

| | | | Composite | | | |
|---|---|---|---|---|---|---|
| | | | of Chothia & | | | |
| Loop | Kabat | Chothia | Kabat | AbM | Contact | IMGT |
| L1 | L24--L34 | L24--L34 | L24--L34 | L24--L34 | L30--L36 | L27-L32 |
| L2 | L50--L56 | L50--L56 | L50--L56 | L50--L56 | L46--L55 | L50-L52 |
| L3 | L89--L97 | L89--L97 | L89--L97 | L89--L97 | L89--L96 | L89-L97 |
| H1 | H31--H35B | H26--H32 . . . H34* | H26--H35B* | H26--H35B | H30--H35B | H26-H33 |
| H2 | H50--H65 | H52--H56 | H50--H65 | H50--H58 | H47--H58 | H51-H56 |
| H3 | H95--H102 | H95--H102 | H95--H102 | H95--H102 | H93--H101 | H93-H102 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H35A is present, it ends at H33. If both H35A and H35B are present, it ends at H34.

The term “antibody” includes intact antibodies and binding fragments thereof. Typically, fragments compete with the intact antibody from which they were derived for specific binding to the target including separate heavy chains, light chains Fab, Fab′, F(ab′)₂, F(ab)c, Dabs, nanobodies, and Fv. Fragments can be produced by recombinant DNA techniques, or by enzymatic or chemical separation of intact immunoglobulins. Antibodies of the present invention also encompass single domain antigen-binding units, which have a camelid scaffold. Animals in the camelid family include camels, llamas, and alpacas. Camelids produce functional antibodies devoid of light chains (IgG2 and IgG3). The heavy chain variable (VH) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies. The term “VHH antibody” or “nanobody” refers to a single variable domain on a heavy chain antibody. A VHH antibody or nanobody is an antigen-binding fragment of a heavy chain only antibody.

The term “antibody” also includes a bispecific antibody and/or a humanized antibody. A bispecific or bifunctional antibody is an artificial hybrid antibody having two different binding sites (see, e.g., Songsivilai and Lachmann, Clin. Exp. Immunol., 79:315-321 (1990); Kostelny et al., J. Immunol., 148:1547-53 (1992)). Some bispecific or bifunctional antibodies have two different heavy/light chain pairs and two different binding sites. Some bispecific or bifunctional antibodies comprise a nanobody binding site and an antibody heavy/light chain binding site. In some bispecific antibodies, one binding site includes a humanized Nb20 mature heavy chain variable region.

In some bispecific antibodies, one binding site includes a humanized Nb20 mature heavy chain variable region as further disclosed below and a heavy chain/light chain pair from an antibody that binds to a receptor expressed on the blood brain barrier, such as an insulin receptor, an insulin-like growth factor (IGF) receptor, a leptin receptor, or a lipoprotein receptor, or a transferrin receptor (Friden et al., Proc. Natl. Acad. Sci. USA 88:4771-4775, 1991; Friden et al., Science 259:373-377, 1993). Such a bispecific antibody can be transferred cross the blood brain barrier by receptor-mediated transcytosis. Brain uptake of the bispecific antibody can be further enhanced by engineering the bispecific antibody to reduce its affinity to the blood brain barrier receptor. Reduced affinity for the receptor resulted in a broader distribution in the brain (see, e.g., Atwal et al., Sci. Trans. Med. 3, 84ra43, 2011; Yu et al., Sci. Trans. Med. 3, 84ra44, 2011).

Bispecific antibodies can also be: (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; or (5) a so-called Scorpion molecule, comprising, e.g., two scFvs fused to both termini of a human Fc-region. Examples of platforms useful for preparing bispecific antibodies include BiTE (Micromet), DART (MacroGenics), Fcab and Mab2 (F-star), Fc-engineered IgGl (Xencor) or DuoBody (based on Fab arm exchange, Genmab).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography or cryogenic electron microscopy (cryo-EM) of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% as measured in a competitive binding assay. Some test antibodies inhibit binding of the references antibody by at least 75%, 90% or 99%. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “pharmaceutically acceptable” means that the carrier, diluent, excipient, or auxiliary is compatible with the other ingredients of the formulation and not substantially deleterious to the recipient thereof.

The term “patient” includes human and other mammalian subjects that receive either prophylactic or therapeutic treatment.

An individual is at increased risk of a disease if the subject has at least one known risk-factor (e.g., genetic, biochemical, family history, and situational exposure) placing individuals with that risk factor at a statistically significant greater risk of developing the disease than individuals without the risk factor.

The term “biological sample” refers to a sample of biological material within or obtainable from a biological source, for example a human or mammalian subject. Such samples can be organs, organelles, tissues, sections of tissues, bodily fluids, peripheral blood, blood plasma, blood serum, cells, molecules such as proteins and peptides, and any parts or combinations derived therefrom. The term biological sample can also encompass any material derived by processing the sample. Derived material can include cells or their progeny. Processing of the biological sample may involve one or more of filtration, distillation, extraction, concentration, fixation, inactivation of interfering components, and the like. Some biological samples are from brain. Some biological samples are brain slices.

The term “control sample” refers to a biological sample not known or suspected to include GPR158-related disease-affected tissue, or at least not known or suspect to include diseased tissues of a given type. Control samples can be obtained from individuals not afflicted with GPR158-related disease, for example individuals not afflicted with an affective disorder, a mood disorder or a brain disorder. Alternatively, control samples can be obtained from patients afflicted with a GPR158-related disease, for example patients afflicted with an affective disorder, a mood disorder or a brain disorder. Such samples can be obtained at the same time as a biological sample thought to comprise the diseased tissue or on a different occasion. A biological sample and a control sample can both be obtained from the same tissue. Preferably, control samples consist essentially or entirely of normal, healthy tissues and can be used in comparison to a biological sample thought to comprise diseased tissue. Preferably, the tissue in the control sample is the same type as the tissue in the biological sample. Preferably, the diseased cells thought to be in the biological sample arise from the same cell type as the type of cells in the control sample.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, as perceived by the subject. A “sign” refers to objective evidence of a disease as observed by a physician.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” or “including” one or more recited elements may include other elements not specifically recited. For example, a composition that “comprises” or “includes” an antibody may contain the antibody alone or in combination with other ingredients. When the disclosure refers to a feature comprising specified elements, the disclosure should alternative be understood as referring to the feature consisting essentially of or consisting of the specified elements. Moreover, elements that are shown or described as being combined with other elements, can, in various embodiments, exist as stand-alone elements.

### DETAILED DESCRIPTION

### I. General

The invention provides antibodies that bind to human GPR158 (mGlyR). Although an understanding of mechanism is not required for practice of the invention, a reduction in an affective disorder, a mood disorder or a brain disorder may occur as a result of the antibody inhibiting GAP activity of GPR158 via RGS7/Gβ5. The antibodies of the invention or agents that induce such antibodies can be used in methods of treating or effecting prophylaxis of an affective disorder, a mood disorder or a brain disorder in a subject. An exemplary antibody is llama Nanobody-20 (Nb20).

In this study, the inventors explored therapeutic relevance of targeting mGlyR as anti-depressant treatment. The inventors have developed specific nanobodies (Nb20) targeting ligand binding Cache domain of mGlyR which mimic the effects of glycine. Through a series of studies, the inventors show that Nb20 alters mGlyR function and its effects on neural circuits producing powerful anti-depressant effects in mouse models when delivered in non-invasive fashion.

In order to identify a ligand capable of modulating GPR158, the inventors screened a library of a llama derived single chain antibody fragments (nanobodies) for specific binding to GPR158. Nanobodies are small fragments of 13-15 kDa that correspond to the variable domains of the heavy chains that recognize antigens. They share the affinity and specificity characteristics of whole immunoglobulins, but are less immunogenic, easier to generate and produce in the prokaryotic system, and can be administered by various routes.

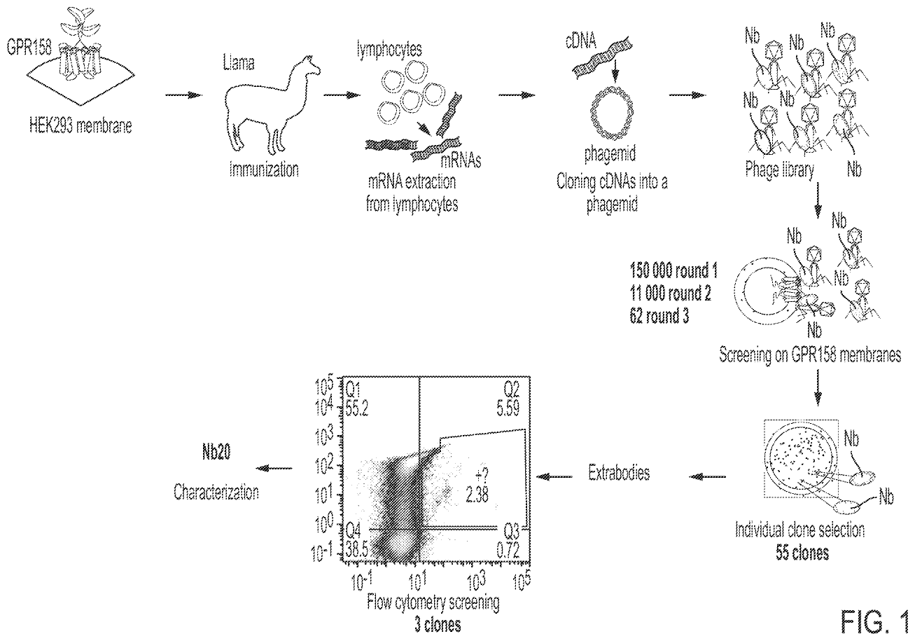

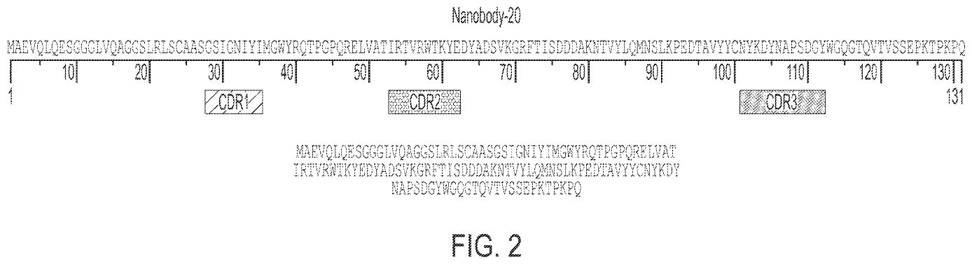

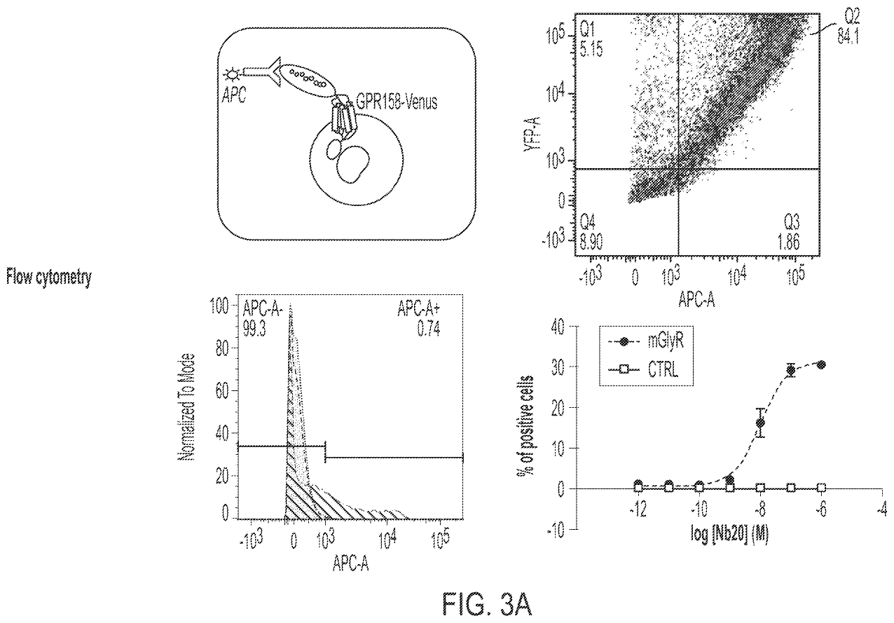

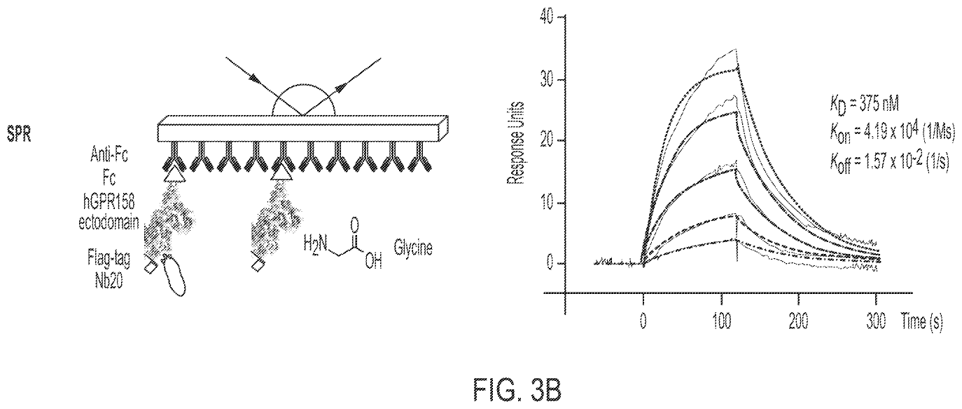

A llama was first immunized with cell membranes expressing GPR158 and then a phage library was created and obtained from INSERM. The library was screened to identify nanobodies binding to GPR158 (FIG. 1). After 3 rounds of amplification 55 clones were further screened in flow cytometry to identify the Nb20 binding specifically GPR158 with a high affinity in whole cell. The Nb20 was sequenced to identify the unique amino acids corresponding to CDR regions by IMGT definition (FIG. 2). Identified Nb20 was tagged with His8 and Myc tags at N-terminus and C-terminus respectively and affinity purified to homogeneity. The resulting purified recombinant Nb20 protein was characterized for binding to full-length GPR158 expressed in HEK293 cells by flow cytometry yielding ~10 nM affinity in concentration titration assays (FIG. 3A). The binding of Nb20 was further characterized using purified extracellular domain of GPR158 (ectodomain) using surface plasmon resonance (SPR) yielding ~375 nM Kd (FIG. 3B).

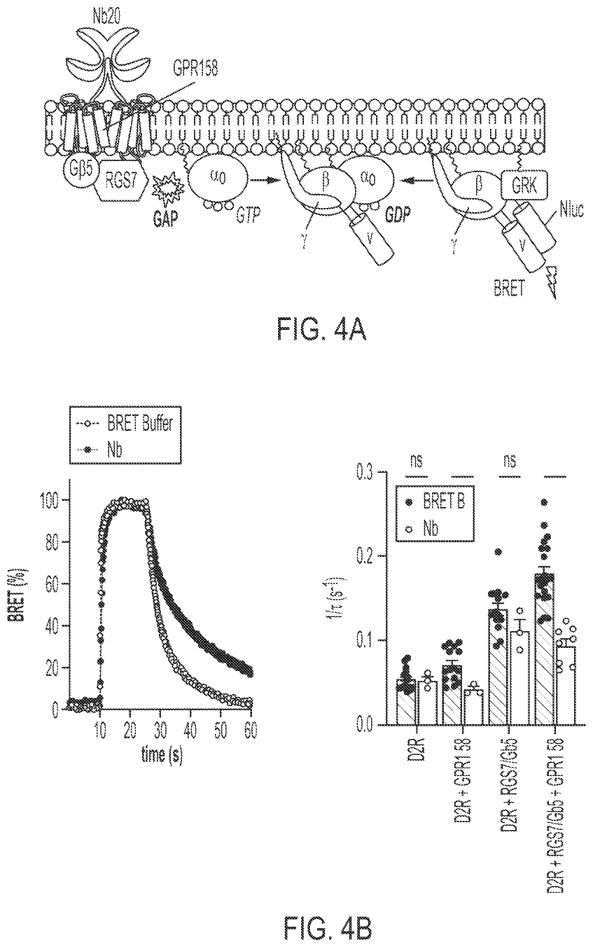

To characterize pharmacological activity of Nb20 the inventors used BRET-based GAP assay where GPR158 was co-expressed with its signal transducer complex RGS7/Gβ5 known to facilitate termination of Goo signaling (FIG. 4A). The inventors found that Nb20 application inhibited the GAP activity of GPR158 via RGS7/Gβ5 (FIG. 4B). Nb20 was found to inhibit GAP activity of GPR158 via RGS7/Gβ5 with an IC50 of least 5.77 nM, N=3. These results indicate that Nb20 antagonizes GPR158 actions and thus is expected to exert anti-depressant effects given that loss of GPR158 activity protects mice from stress-induced depression.

The amino acid sequence of llama nanobody Nb20 is provided as SEQ ID NO:2. Variable region CDRs by IMGT definition are underlined.

(SEQ ID NO: 2)
MAEVQLQESGGGLVQAGGSLRLSCAASGSIGNIYIMGWYRQTPGPQREL
VATIRTVRWTKYEDYADSVKGRFTISDDDAKNTVYLQMNSLKPEDTAVY
YCNYKDYNAPSDGYWGQGTQVTVSSEPKTPKPQ.
Nb20 IMGT CDR-H1 sequence:
(SEQ ID NO: 3)
GSIGNIYI
Nb20 IMGT CDR-H2 sequence:
(SEQ ID NO: 4)
IRTVRWTKYE
Nb20 IMGT CDR-H3 sequence:
(SEQ ID NO: 5)
NYKDYNAPSDGY

Embodiments of the invention are presented in the Examples.

Development of therapies for neuropsychiatric conditions is one of the greatest challenges of modern medicine. Common limitations of traditional small molecule drugs include poor efficacy, off-target side effects and difficult druggability of many targets. In this study, the inventors report a radically different approach deploying small engineered single domain antibodies, known as nanobodies, for the treatment of depression, a prevalent neuropsychiatric condition. The inventors developed highly selective nanobodies for a novel target: the newly discovered glycine receptor mGlyR linked to pathophysiology of depression. Using a mouse model of stress-induced depression, the inventors show that non-invasive intranasal delivery of nanobody produces rapid and lasting anti-depressant effect. The inventors solved an atomic structure of mGlyR bound to nanobody and used a variety of cell-based approaches to reveal the mechanism of mGlyR modulation and its impact on neural circuitry. The antibodies of the invention or agents that induce such antibodies can be used in methods of treating or effecting prophylaxis of an affective disorder, a mood disorder, or a brain disorder in a subject.

In this study the inventors report a unique immunotherapy solution for a major neuropsychiatric condition-depressive disorder. Immunotherapies offer a high degree of specificity, low off target and toxicity and outstanding efficacy and have become a method of choice for treating of cancer and autoimmune disorders (56-57). Immunotherapies are also becoming increasingly applied for brain conditions, most successfully in managing neurodegenerative conditions (58). A few recent studies also indicate their potential utility for managing neuropsychiatric conditions (59-62). In this work the inventors applied an immunotherapy strategy for the first time to effectively suppress depression-related behaviors in mice, thereby uncovering a new direction for therapeutic interventions for a significant health crisis.

The key to the inventors' approach was to develop a single chain antibody, known as nanobody, against the newly discovered metabotropic receptor for glycine, mGlyR, a novel target for developing antidepressants (48). mGlyR's genetic knockout in mice results in marked antidepressant phenotype and resilience to stress-induced depression (29). Molecularly, mGlyR acts non-canonically by engaging RGS7/Gβ5 complex rather than typical G proteins to transduce its signals. Glycine acts as a suppressor of mGlyR signaling thereby relieving the inhibitory influence that RGS7/Gβ5 imposes on G proteins disinhibiting them and allowing signal propagation (48). Consistent with the inhibitory influence of glycine on mGlyR and mGlyR knockout being antidepressant, the inventors find that nanobody Nb20 also inhibits mGlyR and produces antidepressant effect. Detailed structural and mechanistic studies show that Nb20 interacts with the ligand binding Cache domain of mGlyR and inhibit the GAP activity of RGS7/Gβ5 towards its substrate Gao. Thus, Nb20 serves as synthetic antagonist specific for mGlyR and likely tapping into the mechanism of mGlyR antagonism by its endogenous ligand glycine. Manipulating a new target also offers a new modality in the fight to overcome treatment-resistant depression expanding a limited set of currently available options.

Over recent years, nanobodies have been widely deployed as reagents for studying and manipulating GPCRs in the nervous system (63-66). They show several key advantages that make them superb tools including unsurpassed target selectivity, the ability to recognize distinct conformational states, stability, and relatively small size which facilitates target access (67). As a result, nanobodies are increasingly adapted for therapeutic applications to brain disorders (62, 68). The exciting potential of their therapeutic utility is fueled by multiple reports that nanobodies can efficiently reach their targets in the central nervous system. Several mechanisms likely enable this including their active and passive transport across the blood-brain barrier, transcytosis and carrier-assisted delivery (69-72). The inventors show that Nb20 targeting mGlyR shows in vivo antidepressant efficacy when administered intranasally to mice. These observations are similar to previous reports of effective intranasal nanobody delivery (73-74) and suggest that they are readily uptaken by the olfactory neurons. Furthermore, the delivery of nanobodies may be further augmented in depression-related states known to disrupt the blood-brain barrier (75). Overall, the studies support the efficacy of non-invasive nanobody-based therapies for brain disorders and show its effective application for the treatment of depression.

### II. Target Molecules

GPR158, formerly known as an orphan GPCR, is also known as mGlyR. An exemplary amino acid sequence of a human GPR158 (UniProt Q5T848) is provided as SEQ ID NO:1, with the 23 amino acid signal peptide is indicated by boldface. GPR158 comprises a cleavable signal peptide (residues 1-23), an extracellular domain (residues 24-417), a transmembrane domain (residues 418-664), and a cytoplasmic domain (residues 665-1215), numbering with reference to SEQ ID NO:1. The extracellular domain comprises a extracellular cache domain (residues 119 to 310 of SEQ ID NO:1). GPR158 forms homodimers. A GPR158 homodimer is reported to interact with a RGS7-Gβ5 heterodimer (32, 33) Numbering of GPR158 residues is with reference to SEQ ID NO: 1.

(UniProt Q5T848, SEQ ID NO: 1)
MGAMAYPLLLCLLLAQLGLGAVGASRDPQGRPDSPRERTPKGKPHAQQPGRASASD
SSAPWSRSTDGTILAQKLAEEVPMDVASYLYTGDSHQLKRANCSGRYELAGLPGKWPA
LASAHPSLHRALDTLTHATNFLNVMLQSNKSREQNLQDDLDWYQALVWSLLEGEPSIS
RAAITFSTDSLSAPAPQVFLQATREESRILLQDLSSSAPHLANATLETEWFHGLRRKWRP
HLHRRGPNQGPRGLGHSWRRKDGLGGDKSHFKWSPPYLECENGSYKPGWLVTLSSAIY
GLQPNLVPEFRGVMKVDINLQKVDIDQCSSDGWFSGTHKCHLNNSECMPIKGLGFVLG
AYECICKAGFYHPGVLPVNNFRRRGPDQHISGSTKDVSEEAYVCLPCREGCPFCADDSP
CFVQEDKYLRLAIISFQALCMLLDFVSMLVVYHFRKAKSIRASGLILLETILFGSLLLYFP
VVILYFEPSTFRCILLRWARLLGFATVYGTVTLKLHRVLKVFLSRTAQRIPYMTGGRVM
RMLAVILLVVFWFLIGWTSSVCQNLEKQISLIGQGKTSDHLIFNMCLIDRWDYMTAVAE
FLFLLWGVYLCYAVRTVPSAFHEPRYMAVAVHNELIISAIFHTIRFVLASRLQSDWMLM
LYFAHTHLTVTVTIGLLLIPKFSHSSNNPRDDIATEAYEDELDMGRSGSYLNSSINSAWSE
HSLDPEDIRDELKKLYAQLEIYKRKKMITNNPHLQKKRCSKKGLGRSIMRRITEIPETVSR
QCSKEDKEGADHGTAKGTALIRKNPPESSGNTGKSKEETLKNRVFSLKKSHSTYDHVRD
QTEESSSLPTESQEEETTENSTLESLSGKKLTQKLKEDSEAESTESVPLVCKSASAHNLSS
EKKTGHPRTSMLQKSLSVIASAKEKTLGLAGKTQTAGVEERTKSQKPLPKDKETNRNHS
NSDNTETKDPAPQNSNPAEEPRKPQKSGIMKQQRVNPTTANSDLNPGTTQMKDNFDIGE
VCPWEVYDLTPGPVPSESKVQKHVSIVASEMEKNPTFSLKEKSHHKPKAAEVCQQSNQ
KRIDKAEVCLWESQGQSILEDEKLLISKTPVLPERAKEENGGQPRAANVCAGQSEELPPK
AVASKTENENLNQIGHQEKKTSSSEENVRGSYNSSNNFQQPLTSRAEVCPWEFETPAQP
NAGRSVALPASSALSANKIAGPRKEEIWDSFKV

Unless otherwise apparent from the context, reference to GPR158 means a natural human form of GPR158 including all isoforms, including soluble forms, irrespective of whether posttranslational modification (e.g., phosphorylation, glycation, or acetylation) is present. Reference to GPR158 includes known natural variations which are listed in the Swiss-Prot database and permutations thereof, as well as mutations associated with pathologies.

Additionally, reference to GPR158 includes GPR158 with known post-translational modifications. Examples of known post-translational modifications are listed in the UniProtKB/Swiss-Prot database. Unless otherwise apparent from context, reference to GPR158, or its fragments includes the natural human amino acid sequences including isoforms, mutants, and allelic variants thereof.

### III. Antibodies

### A. Binding Specificity and Functional Properties

The invention provides antibodies that bind to GPR158. These antibodies can be obtained by immunizing with a GPR158 polypeptide purified from a natural source or recombinantly expressed. Antibodies can be screened for binding GPR158. Nb20 is an example of a monoclonal antibody binding human GPR158. The invention also provides antibodies binding to the same epitope as any of the foregoing antibodies, such as, for example, the epitope of Nb20. Also included are antibodies competing for binding to GPR158 with any of the foregoing antibodies, such as, for example, competing with Nb20.

The above-mentioned antibodies can be generated de novo by immunizing with a full length GPR158 polypeptide or peptide fragment thereof or with cell membranes expressing human GPR158 or a peptide fragment thereof. In some embodiments, peptides are attached to a heterologous conjugate molecule that helps elicit an antibody response to the peptide. Attachment can be direct or via a spacer peptide or amino acid. Cysteine is used as a spacer amino acid because its free SH group facilitates attachment of a carrier molecule. A polyglycine linker (e.g., 2-6 glycines), with or without a cysteine residue between the glycines and the peptide can also be used. The carrier molecule serves to provide a T-cell epitope that helps elicit an antibody response against the peptide. Several carriers are commonly used particularly keyhole limpet hemocyanin (KLH), ovalbumin and bovine serum albumin (BSA). Peptide spacers can be added to peptide immunogen as part of solid phase peptide synthesis. Carriers are typically added by chemical cross-linking. Some examples of chemical crosslinkers that can be used include cross-N-maleimido-6-aminocaproyl ester or m-maleimidobenzoyl-N-hydroxysuccinimide ester (MBS) (see for example, Harlow, E. et al., Antibodies: A Laboratory Manual, Cold Spring Harbor Laboratory Press, Cold Spring Harbor, N.Y. 1988; Sinigaglia et al., Nature, 336:778-780 (1988); Chicz et al., J. Exp. Med., 178:27-47 (1993); Hammer et al., Cell 74:197-203 (1993); Falk K. et al., Immunogenetics, 39:230-242 (1994); WO 98/23635; and, Southwood et al. J. Immunology, 160:3363-3373 (1998)). The carrier and spacer if present can be attached to either end of the immunogen.

A peptide with optional spacer and carrier can be used to immunize laboratory animals or B-cells as described in more detail below. Hybridoma supernatants can be tested for ability to bind GPR158 or a peptide fragment thereof. The peptide can be attached to a carrier or other tag to facilitate the screening assay. In this case, the carrier or tag is preferentially different than the combination of spacer and carrier molecule used for immunization to eliminate antibodies specific for the spacer or carrier rather than the GPR158 peptide. Any of the GPR158 isoforms can be used. Cell membranes or cells expressing human GPR158 or a fragment thereof may be used in binding studies.

The invention provides monoclonal antibodies binding to epitopes within GPR158. An antibody designated Nb20 is one such exemplary llama antibody. Unless otherwise apparent from context, reference to Nb20 should be understood as referring to any of the llama, chimeric, veneered, and humanized forms of this antibody. This antibody is further characterized by its ability to bind full-length GPR158 expressed in HEK293 cells by flow cytometry with at least 10 nM affinity, its ability to bind purified extracellular domain of GPR158 (ectodomain) using surface plasmon resonance (SPR) with at least 375 nM Kd, and its ability to inhibit GAP activity of GPR158 via RGS7/Gβ5 with an IC50 of least 5.77 nM.

Some antibodies of the invention bind to the same or overlapping epitope as an antibody designated Nb20. The sequence of the heavy chain mature variable region of this antibody is designated SEQ ID NO:2. Other antibodies having such a binding specificity can be produced by immunizing mice with GPR158 or a portion thereof including the desired epitope and screening resulting antibodies for binding to GPR158 optionally in competition with an antibody having the heavy chain variable region of llama Nb20. Fragments of GPR158 including the desired epitope can be linked to a carrier that helps elicit an antibody response to the fragment and/or be combined with an adjuvant the helps elicit such a response. Such antibodies can be screened for differential binding to GPR158 or a fragment thereof compared with mutants of specified residues. Screening against such mutants more precisely defines the binding specificity to allow identification of antibodies whose binding is inhibited by mutagenesis of particular residues and which are likely to share the functional properties of other exemplified antibodies. The mutations can be systematic replacement substitution with alanine (or serine if an alanine is present already) one residue at a time, or more broadly spaced intervals, throughout the target or throughout a section thereof in which an epitope is known to reside. If the same set of mutations significantly reduces the binding of two antibodies, the two antibodies bind the same epitope.

Antibodies having the binding specificity of a selected llama antibody (e.g., Nb20) can also be produced using a variant of the phage display method. See Winter, WO 92/20791. This method is particularly suitable for producing human antibodies. In this method, either the heavy or light chain variable region of the selected non-human antibody is used as a starting material. If, for example, a light chain variable region is selected as the starting material, a phage library is constructed in which members display the same light chain variable region (i.e., the non-human starting material) and a different heavy chain variable region. The heavy chain variable regions can for example be obtained from a library of rearranged human heavy chain variable regions. A phage showing strong specific binding for GPR158 or a fragment thereof (e.g., at least 108 and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for GPR158 or a fragment thereof are selected. The resulting antibodies usually have the same or similar epitope specificity as the non-human starting material. Antibodies having the binding specificity of a selected llama antibody (e.g., Nb20) can be produced, for example, by a variant of the M13-phage display method (+phage helper KM13), where a camelid single-chain antibody is used as starting material (M13-phage, and phage helper KM13, see Peltomaa, R., et al., ACS Omega 4, 11569-11580 (2019)). The resulting antibodies usually have the same or similar epitope specificity as the llama starting material.

Other antibodies can be obtained by mutagenesis of cDNA encoding the heavy chain of an exemplary antibody, such as Nb20. Monoclonal antibodies that are at least 70%, 80%, 90%, 95%, 96%, 97%, 98%, or 99% identical to Nb20 in amino acid sequence of the mature heavy chain variable region and maintain its functional properties, and/or which differ from the respective antibody by a small number of functionally inconsequential amino acid substitutions (e.g., conservative substitutions), deletions, or insertions are also included in the invention. Monoclonal antibodies having at least one, two, or all three CDR(s) as defined by any conventional definition, but preferably IMGT, that are 90%, 95%, 99% or 100% identical to corresponding CDRs of Nb20 are also included.

The invention also provides antibodies having some or all (e.g., 1, 2, or 3) CDRs entirely or substantially from Nb20. Such antibodies can include a heavy chain variable region that has at least two, and usually all three, CDRs entirely or substantially from the heavy chain variable region of Nb20. The antibodies can include both heavy and light chains. A CDR is substantially from a corresponding Nb20 CDR when it contains no more than 4, 3, 2, or 1 substitutions, insertions, or deletions, except that CDR-H2 (when defined by Kabat) can have no more than 6, 5, 4, 3, 2, or 1 substitutions, insertions, or deletions. Such antibodies can have at least 70%, 80%, 90%, 95%, 96%, 97%, 98%, or 99% identity to Nb20 in the amino acid sequence of the mature heavy chain variable region and maintain their functional properties, and/or differ from Nb20 by a small number of functionally inconsequential amino acid substitutions (e.g., conservative substitutions), deletions, or insertions.

Some antibodies identified by such assays can bind to human, mouse or mammalian GPR158 or a peptide fragment thereof. Some antibodies identified by such assays can bind to cell membranes and/or cells expressing human, mouse or mammalian GPR158 or a peptide fragment thereof.

Antibodies can be screened for binding to GPR158 in cell-based assays (whole cells or membrane) or in a protein-based assay. Cells expressing recombinant GPR158 on their surface may be used in flow cytometry assays (see, for example, FIG. 1 and FIG. 3A), radioligand binding assays, or microscopy-based assays. Exemplary cells useful in cell-based assays are HEK293 cells expressing recombinant GPR158 on their surface. Protein-based assays using GPR158 or a fragment thereof, for example a recombinant cache domain protein, include Surface Plasmon Resonance (SPR) (see, for example, FIG. 3B), BioLayer Interferometry (BLI), Isothermal Titration calorimetry (ITC, NanoITC), or coimmunoprecipitation assays.

### B. Non-Human Antibodies

The production of other non-human antibodies, e.g., llama, alpaca, camel, murine, guinea pig, primate, rabbit or rat, against GPR158 or a fragment thereof can be accomplished by, for example, immunizing the animal with GPR158 or a fragment thereof, or cell membranes expressing GPR158 or a fragment thereof. See Harlow & Lane, Antibodies, A Laboratory Manual (CSHP NY, 1988) (incorporated by reference for all purposes). Such an immunogen can be obtained from a natural source, by peptide synthesis, or by recombinant expression. Optionally, the immunogen can be administered fused or otherwise complexed with a carrier protein. Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to GPR158 or an epitope within GPR158. Such screening can be accomplished by determining binding of an antibody to a collection of GPR158 variants, and determining which GPR158 variants bind to the antibody. Binding can be assessed, for example, by Western blot, flow cytometry or ELISA.

### C. Humanized Antibodies

A humanized antibody is a genetically engineered antibody in which CDRs from a non-human “donor” antibody are grafted into human “acceptor” antibody sequences or into acceptor humanized nanobody scaffolds (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; Foote, U.S. Pat. No. 6,881,557, Vincke, C. et al., (2009) J. Biol. Chem. 284 (5): 3273-3284, and Sulea, T. (2022). Humanization of Camelid Single-Domain Antibodies. In: Hussack, G., Henry, K. A. (eds) Single-Domain Antibodies. Methods in Molecular Biology, vol 2446. Humana, New York, NY). The acceptor antibody sequences can be, for example, a mature human antibody sequence, a composite of such sequences, a consensus sequence of human antibody sequences, or a germline region sequence. Thus, a humanized antibody is an antibody having at least three, four, five or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A humanized nanobody can comprise llama nanobody heavy chain CDRs grafted into an acceptor humanized VHH nanobody scaffold, for example h-NbBcII10_{FGLA} (Vincke, supra). A CDR in a humanized antibody is substantially from a corresponding CDR in a non-human antibody when at least 85%, 90%, 95% or 100% of corresponding residues (as defined by any conventional definition but preferably defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical. To be classified as humanized under the 2014 World Health Organization (WHO) International non-proprietary names (INN) definition of humanized antibodies, an antibody must have at least 85% identity to human germline antibody sequences (i.e., prior to somatic hypermutation). Mixed antibodies are antibodies for which one antibody chain (e.g., heavy chain) meets the threshold but the other chain (e.g., light chain) does not meet the threshold. An antibody is classified as chimeric if neither chain meets the threshold, even though the variable framework regions for both chains were substantially human with some non-human backmutations. See, Jones et al. (2016) The INNs and outs of antibody nonproprietary names, mAbs 8:1, 1-9, DOI: 10.1080/19420862.2015.1114320. See also “WHO-INN: International nonproprietary names (INN) for biological and biotechnological substances (a review)” (Internet) 2014. Available from: world wide web.who.int/medicines/services/inn/BioRev2014.pdf), incorporated herein by reference. For the avoidance of doubt, the term “humanized” as used herein is not intended to be limited to the 2014 WHO INN definition of humanized antibodies. Some of the humanized antibodies provided herein have at least 85% sequence identity to human germline sequences and some of the humanized antibodies provided herein have less than 85% sequence identity to human germline sequences. Some of the heavy chains of the humanized antibodies provided herein have from about 60% to 100% sequence identity to human germ line sequences, such as, for example, in the range of about 60% to 69%, 70% to 79%, 80% to 84%, or 85% to 89%. Some heavy chains fall below the 2014 WHO INN definition and have, for example, about 64%, 65%, 66%, 67%, 68%, 69%, 70%, 71%, 72%, 73%, 74%, 75%, 76%, 77%, 78%, 79%, 80%, 81%, or 82%, 83%, or 84% sequence identity to human germ line sequences, while other heavy chains meet the 2014 WHO INN definition and have about 85%, 86%, 87%, 88%, 89% or greater sequence identity to human germ line sequences. Some of the light chains of the humanized antibodies provided herein have from about 60% to 100% sequence identity to human germ line sequences, such as, for example, in the range of about 80% to 84% or 85% to 89%. Some light chains fall below the 2014 WHO INN definition and have, for example, about 81%, 82%, 83% or 84% sequence identity to human germ line sequences, while other light chains meet the 2014 WHO INN definition and have about 85%, 86%, 87%, 88%, 89% or greater sequence identity to human germ line sequences. Some humanized antibodies provided herein that are “chimeric” under the 2014 WHO INN definition have heavy chains with less than 85% identity to human germ line sequences paired with light chains having less than 85% identity to human germ line sequences. Some humanized antibodies provided herein are “mixed” under the 2014 WHO INN definition, for example, having a heavy chain with at least 85% sequence identity to human germ line sequences paired with a light chain having less than 85% sequence identity to human germ line sequences, or vice versa. Some humanized antibodies provided herein meet the 2014 WHO INN definition of “humanized” and have a heavy chain with at least 85% sequence identity to human germ line sequences paired with a light chain having at least 85% sequence identity to human germ line sequences. Additional humanized antibodies of the invention meet the 2014 WHO INN definition of “mixed”.

Although humanized antibodies often incorporate all six CDRs (defined by any conventional definition but preferably as defined by Kabat) from a non-human antibody, they can also be made with less than all CDRs (e.g., at least 3, 4, or 5 CDRs) from a non-human antibody (e.g., Pascalis et al., J. Immunol. 169:3076, 2002; Vajdos et al., J. of Mol. Biol., 320:415-428, 2002; Iwahashi et al., Mol. Immunol. 36:1079-1091, 1999; Tamura et al, J. Immunol., 164:1432-1441, 2000). Some humanized antibodies incorporate one, two, or three heavy chain CDRs from a camelid single-domain antibody (see, e.g., Vincke, supra and Sulea, T., supra). Some humanized antibodies incorporate one, two, or three heavy chain CDRs from llama nanobody Nb20. Some humanized antibodies comprise one, two, or three heavy chain CDRs from Nb20 grafted into an acceptor humanized VHH nanobody scaffold, for example h-NbBcII10_{FGLA} (Vincke, supra).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41:863, 2004. In such humanized antibodies at positions in which one or more donor CDR residues is absent or in which an entire donor CDR is omitted, the amino acid occupying the position can be an amino acid occupying the corresponding position (by Kabat numbering) in the acceptor antibody or acceptor humanized nanobody scaffold sequence. The number of such substitutions of acceptor for donor amino acids in the CDRs to include reflects a balance of competing considerations. Such substitutions are potentially advantageous in decreasing the number of llama amino acids in a humanized antibody and consequently decreasing potential immunogenicity and/or for meeting the WHO INN definition of “humanized”. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

If more than one human acceptor antibody sequence is selected, a composite or hybrid of those acceptors can be used, and the amino acids used at different positions in the humanized light chain and heavy chain variable regions can be taken from any of the human acceptor antibody sequences used.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a non-human variable region framework residue and a selected human variable region framework residue, the human framework amino acid can be substituted by the equivalent framework amino acid from the non-human antibody when it is reasonably expected that the amino acid:
(1) noncovalently binds antigen directly;
(2) is adjacent to a CDR region or within a CDR as defined by Chothia but not Kabat;
(3) otherwise interacts with a CDR region (e.g., is within about 6 Å of a CDR region), (e.g., identified by modeling the light or heavy chain on the solved structure of a homologous known immunoglobulin chain); or
(4) is a residue participating in the VL-VH interface.

In an embodiment, humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682)].

Framework residues from classes (1) through (3) as defined by Queen, U.S. Pat. No. 5,530,101, are sometimes alternately referred to as canonical and vernier residues. Framework residues that help define the conformation of a CDR loop are sometimes referred to as canonical residues (Chothia & Lesk, J. Mol. Biol. 196:901-917 (1987); Thornton & Martin, J. Mol. Biol. 263:800-815 (1996)). Framework residues that support antigen-binding loop conformations and play a role in fine-tuning the fit of an antibody to antigen are sometimes referred to as vernier residues (Foote & Winter, J. Mol. Biol 224:487-499 (1992)).

Other framework residues that are candidates for substitution are residues creating a potential glycosylation site. Still other candidates for substitution are acceptor human framework amino acids that are unusual for a human immunoglobulin at that position. These amino acids can be substituted with amino acids from the equivalent position of the llama donor antibody or from the equivalent positions of more typical human immunoglobulins.

Other framework residues that are candidates for substitution are N-terminal glutamine residues (Q) that may be replaced with glutamic acid (E) to minimize potential for pyroglutamate conversion [Y. Diana Liu, et al., 2011, J. Biol. Chem., 286:11211-11217]. Glutamic acid (E) conversion to pyroglutamate (pE) occurs more slowly than from glutamine (Q). Because of the loss of a primary amine in the glutamine to pE conversion, antibodies become more acidic. Incomplete conversion produces heterogeneity in the antibody that can be observed as multiple peaks using charge-based analytical methods. Heterogeneity differences may indicate a lack of process control.

The llama nanobody Nb20 comprises a mature heavy chain variable region having amino acid sequence comprising SEQ ID NO:2.

In some humanized Nb20 antibodies, the variable heavy chain has ≥85% identity to human sequence.

The CDR regions of such humanized antibodies can be identical or substantially identical to the CDR regions of Nb20. The CDR regions can be defined by any conventional definition (e.g., Kabat, Chothia, Kabat/Chothia Composite, AbM, Contact, or IMGT) but are preferably as defined by IMGT.

Variable region framework positions are in accordance with Kabat numbering unless otherwise stated.

A possibility for additional variation in humanized Nb20 variants is additional backmutations in the variable region frameworks. Many of the framework residues not in contact with the CDRs in the humanized mAb can accommodate substitutions of amino acids from the corresponding positions of the donor llama mAb or other llama or human antibodies, and even many potential CDR-contact residues are also amenable to substitution. Even amino acids within the CDRs may be altered, for example, with residues found at the corresponding position of the human acceptor sequence or acceptor humanized nanobody scaffold sequence used to supply variable region frameworks. In addition, alternate human acceptor sequences can be used, for example, for the heavy and/or light chain. In addition, alternate acceptor humanized nanobody scaffold sequences can be used, for example, for the heavy chain. If different acceptor sequences or different acceptor humanized nanobody scaffold sequences are used, one or more of the backmutations recommended above may not be performed because the corresponding donor and acceptor residues are already the same without backmutations.

Preferably, replacements or backmutations in humanized Nb20 variants (whether or not conservative) have no substantial effect on the binding affinity or potency of the humanized mAb, that is, its ability to bind to full-length GPR158, its ability to bind to extracellular domain of GPR158, and/or its ability to inhibit GAP activity of GPR158 via RGS7/Gβ5. The humanized Nb20 antibodies are further characterized by their ability to inhibit GAP activity of GPR158 via RGS7/Gβ5.

### D. Chimeric and Veneered Antibodies

The invention further provides chimeric and veneered forms of non-human antibodies, particularly of Nb20 antibody.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of the Nb20 antibody are included in the invention.

### E. Human Antibodies

Human antibodies against GPR158 or a fragment thereof are provided by a variety of techniques described below. Some human antibodies are selected by competitive binding experiments, by the phage display method of Winter, above, or otherwise, to have the same epitope specificity as a particular llama antibody, such nanobody Nb20. Human antibodies can also be screened for a particular epitope specificity by using only a fragment of GPR158 as the target antigen, and/or by screening antibodies against a collection of GPR158 variants.

Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397; 5,874,299; 5,814,318; 5,789,650; 5,770,429; 5,661,016; 5,633,425; 5,625,126; 5,569,825; 5,545,806; Neuberger, Nat. Biotechnol. 14:826 (1996); and Kucherlapati, WO 91/10741 (1991)) phage display methods (see, e.g., Dower et al., WO 91/17271; McCafferty et al., WO 92/01047; U.S. Pat. Nos. 5,877,218; 5,871,907; 5,858,657; 5,837,242; 5,733,743; and 5,565,332); and methods described in WO 2008/081008 (e.g., immortalizing memory B cells isolated from humans, e.g., with EBV, screening for desired properties, and cloning and expressing recombinant forms).

### F. Selection of Constant Region

The heavy and light chain variable regions of chimeric, veneered or humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Human IgG1 and IgG3 also induce stronger cell mediated effector functions than human IgG2 and IgG4. Light chain constant regions can be lambda or kappa. Numbering conventions for constant regions include EU numbering (Edelman, G. M. et al., Proc. Natl. Acad. USA, 63, 78-85 (1969)), Kabat numbering (Kabat, Sequences of Proteins of Immunological Interest (National Institutes of Health, Bethesda, MD, 1991, IMGT unique numbering (Lefranc M.-P. et al., IMGT unique numbering for immunoglobulin and T cell receptor constant domains and Ig superfamily C-like domains, Dev. Comp. Immunol., 29, 185-203 (2005), and IMGT exon numbering (Lefranc, supra).

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduce affinity for Fcγ receptors, particularly FcγRI receptor (see, e.g., U.S. Pat. No. 6,624,821). An alanine substitution at positions 234, 235, and 237 of human IgG1 can be used for reducing effector functions. Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821). In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine. In some antibodies, the isotype is human IgG2, IgG3, or IgG4.

Antibodies can be expressed as tetramers containing two light and two heavy chains, as separate heavy chains, light chains, as Fab, Fab′, F(ab′)2, and Fv, or as single chain antibodies in which heavy and light chain mature variable domains are linked through a spacer. Antibodies can be expressed as a single chain antibody or nanobody comprising a heavy chain variable region.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Thus, for example, another heavy chain constant region is of IgG1 G1m3 with or without the C-terminal lysine. Reference to a human constant region includes a constant region with any natural allotype or any permutation of residues occupying positions in natural allotypes.

### G. Expression of Recombinant Antibodies

A number of methods are known for producing chimeric and humanized antibodies using an antibody-expressing cell line (e.g., hybridoma). For example, the immunoglobulin variable regions of antibodies can be cloned and sequenced using well known methods. In one method, the heavy chain variable VH region is cloned by RT-PCR using mRNA prepared from hybridoma cells. Consensus primers are employed to the VH region leader peptide encompassing the translation initiation codon as the 5′ primer and a g2b constant regions specific 3′ primer. Exemplary primers are described in U.S. patent publication US 2005/0009150 by Schenk et al. (hereinafter “Schenk”). The sequences from multiple, independently derived clones can be compared to ensure no changes are introduced during amplification. The sequence of the VH region can also be determined or confirmed by sequencing a VH fragment obtained by 5′ RACE RT-PCR methodology and the 3′ g2b specific primer.

The light chain variable VL region can be cloned in an analogous manner. In one approach, a consensus primer set is designed for amplification of VL regions using a 5′ primer designed to hybridize to the VL region encompassing the translation initiation codon and a 3′ primer specific for the Ck region downstream of the V-J joining region. In a second approach, 5′RACE RT-PCR methodology is employed to clone a VL encoding cDNA. Exemplary primers are described in Schenk, supra. The cloned sequences are then combined with sequences encoding human (or other non-human species) constant regions.

In one approach, the heavy and light chain variable regions are re-engineered to encode splice donor sequences downstream of the respective VDJ or VJ junctions and are cloned into a mammalian expression vector, such as pCMV-hγ1 for the heavy chain and pCMV-Mcl for the light chain. These vectors encode human γ1 and Ck constant regions as exonic fragments downstream of the inserted variable region cassette. Following sequence verification, the heavy chain and light chain expression vectors can be co-transfected into CHO cells to produce chimeric antibodies. Conditioned media is collected 48 hours post-transfection and assayed by western blot analysis for antibody production or ELISA or flow cytometry for antigen binding. The chimeric antibodies are humanized as described above.

Chimeric, veneered, humanized, and human antibodies are typically produced by recombinant expression. Recombinant polynucleotide constructs typically include an expression control sequence operably linked to the coding sequences of antibody chains, including naturally associated or heterologous expression control elements, such as a promoter. The expression control sequences can be promoter systems in vectors capable of transforming or transfecting eukaryotic or prokaryotic host cells. Once the vector has been incorporated into the appropriate host, the host is maintained under conditions suitable for high level expression of the nucleotide sequences and the collection and purification of the crossreacting antibodies.

These expression vectors are typically replicable in the host organisms either as episomes or as an integral part of the host chromosomal DNA. Commonly, expression vectors contain selection markers, e.g., ampicillin resistance or hygromycin resistance, to permit detection of those cells transformed with the desired DNA sequences.

E. coli is one prokaryotic host useful for expressing antibodies, particularly antibody fragments. E. coli BL21 DE3±pLysS is an exemplary prokaryotic host. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NSO. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride or heat shock transformation is commonly utilized for prokaryotic cells, whereas calcium phosphate treatment, electroporation, lipofection, biolistics, or viral-based transfection can be used for other cellular hosts. Other methods used to transfect mammalian cells include the use of polybrene, protoplast fusion, liposomes, electroporation, and microinjection. For production of transgenic animals, transgenes can be microinjected into fertilized oocytes or can be incorporated into the genome of embryonic stem cells, and the nuclei of such cells transferred into enucleated oocytes.

Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivities above 50 μg or 100 μg per cell per day, which correspond to product titers of greater than 7.5 g/L culture, can be used. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, antibodies can be purified according to standard procedures of the art, including protein A capture, nickel column capture, HPLC purification, column chromatography, gel electrophoresis and the like (see generally, Scopes, Protein Purification (Springer-Verlag, NY, 1982)).

Methodology for commercial production of antibodies can be employed, including codon optimization, selection of promoters, selection of transcription elements, selection of terminators, serum-free single cell cloning, cell banking, use of selection markers for amplification of copy number, CHO terminator, or improvement of protein titers (see, e.g., U.S. Pat. Nos. 5,786,464; 6,114,148; 6,063,598; 7,569,339; WO2004/050884; WO2008/012142; WO2008/012142; WO2005/019442; WO2008/107388; WO2009/027471; and U.S. Pat. No. 5,888,809).

### IV. Active Immunogens

An agent used for active immunization serves to induce in a patient the same types of antibody described in connection with passive immunization above. Agents used for active immunization can be the same types of immunogens used for generating monoclonal antibodies in laboratory animals, e.g., a peptide of 3-15 or 3-12 or 5-12, or 5-8 contiguous amino acids from a region of GPR158. For inducing antibodies binding to the same or overlapping epitope as Nb20, the epitope specificity of these antibodies can be mapped (e.g., by testing binding to a series of overlapping peptides spanning GPR158). A fragment of GPR158 consisting of or including or overlapping the epitope can then be used as an immunogen. Some active agents are cell membranes expressing GPR158 or fragment thereof.

The heterologous carrier and adjuvant, if used may be the same as used for generating monoclonal antibody, but may also be selected for better pharmaceutical suitability for use in humans. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-S-glycerine cysteine (Pam3Cys), mannan (a mannose polymer), or glucan (a β 1->2 polymer)), cytokines (e.g., IL-1, IL-1 alpha and β peptides, IL-2, γ-INF, IL-10, GM-CSF), and chemokines (e.g., MIP1-α and β, and RANTES). Immunogens may be linked to the carriers with or without spacers amino acids (e.g., gly-gly). Additional carriers include virus-like particles. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLOS ONE 2 (5): e415.) Alternatively, peptide immunogens can be linked to at least one artificial T-cell epitope capable of binding a large proportion of MHC Class II molecules., such as the pan DR epitope (“PADRE”). PADRE is described in U.S. Pat. No. 5,736,142, WO 95/07707, and Alexander J et al, Immunity, 1:751-761 (1994). Active immunogens can be presented in multimeric form in which multiple copies of an immunogen and/or its carrier are presented as a single covalent molecule.

Fragments are often administered with pharmaceutically acceptable adjuvants. The adjuvant increases the titer of induced antibodies and/or the binding affinity of induced antibodies relative to the situation if the peptide were used alone. A variety of adjuvants can be used in combination with an immunogenic fragment of GPR158 to elicit an immune response. Preferred adjuvants augment the intrinsic response to an immunogen without causing conformational changes in the immunogen that affect the qualitative form of the response. Preferred adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA; now Antigenics, Inc., New York, NY). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Another adjuvant is CpG (WO 98/40100). Adjuvants can be administered as a component of a therapeutic composition with an active agent or can be administered separately, before, concurrently with, or after administration of the therapeutic agent.

Analogs of natural fragments of GPR158 that induce antibodies against GPR158 can also be used. For example, one or more or all L-amino acids can be substituted with D amino acids in such peptides. Also the order of amino acids can be reversed (retro peptide). Optionally a peptide includes all D-amino acids in reverse order (retro-inverso peptide). Peptides and other compounds that do not necessarily have a significant amino acid sequence similarity with GPR158 peptides but nevertheless serve as mimetics of GPR158 peptides and induce a similar immune response. Anti-idiotypic antibodies against monoclonal antibodies to GPR158 as described above can also be used. Such anti-Id antibodies mimic the antigen and generate an immune response to it (see Essential Immunology, Roit ed., Blackwell Scientific Publications, Palo Alto, CA 6th ed., p. 181).

Peptides (and optionally a carrier fused to the peptide) can also be administered in the form of a nucleic acid encoding the peptide and expressed in situ in a patient. A nucleic acid segment encoding an immunogen is typically linked to regulatory elements, such as a promoter and enhancer that allow expression of the DNA segment in the intended target cells of a patient. For expression in blood cells, as is desirable for induction of an immune response, promoter and enhancer elements from light or heavy chain immunoglobulin genes or the CMV major intermediate early promoter and enhancer are suitable to direct expression. The linked regulatory elements and coding sequences are often cloned into a vector. Antibodies can also be administered in the form of nucleic acids encoding the antibody heavy and/or light chains. If both heavy and light chains are present, the chains are preferably linked as a single chain antibody. Antibodies for passive administration can also be prepared e.g., by affinity chromatography from sera of patients treated with peptide immunogens.

The DNA can be delivered in naked form (i.e., without colloidal or encapsulating materials). Alternatively a number of viral vector systems can be used including retroviral systems (see, e.g., Lawrie and Tumin, Cur. Opin. Genet. Develop. 3, 102-109 (1993)); adenoviral vectors {see, e.g., Bett et al, J. Virol. 67, 5911 (1993)); adeno-associated virus vectors {see, e.g., Zhou et al., J. Exp. Med. 179, 1867 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333 (1995); Woo et al, WO 94/12629 and Xiao & Brandsma, Nucleic Acids. Res. 24, 2630-2622 (1996)).

DNA encoding an immunogen, or a vector containing the same, can be packaged into liposomes. Suitable lipids and related analogs are described by U.S. Pat. Nos. 5,208,036, 5,264,618, 5,279,833, and 5,283,185. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. 1996).

### V. Antibody Screening Assays

Antibodies can be initially screened for the intended binding specificity as described above. Active immunogens can likewise be screened for capacity to induce antibodies with such binding specificity. In this case, an active immunogen is used to immunize a laboratory animal and the resulting sera tested for the appropriate binding specificity.

Antibodies having the desired binding specificity can then be tested in cellular and animal models, for example in ex vivo brain slices from animals expressing GPR158.

### A. Cell-Based Assays:

Antibodies can be screened in cell-based assays for effects on GPR158 signaling activity, for example by measuring CAMP levels or GAP activity of RGS7-Gβ5 modulated by GPR158. Additionally designed assays may rely on measuring conformational changes induced by antibody binding by placing fluorescent resonance energy transfer (FRET) or Bioluminescence Resonance Energy Transfer (BRET) donor/acceptor pairs within GPR158-RGS7-Gβ5 complex.

An exemplary cell-based assay measures the effects of an antibody on cAMP production using a Bioluminescence Resonance Energy Transfer (BRET)-based CAMP biosensor in cells expressing GPR158. CAMYEL (CAMP sensor using YFP-Epac-Rluc) is a unimolecular BRET-based biosensor for cAMP activity, consisting of truncated and catalytically inactive human Epac1 sandwiched between Rluc (the donor) and a monomeric and circularly permuted form of the YFP citrine (the acceptor). When CAMP is not present, Epac adopts a “closed” conformation, where the donor and acceptor are in close proximity, producing a BRET signal. When CAMP binds to Epac, the donor and acceptor move farther apart, reducing BRET (Valkovic, A. L. et al., Pharmacol Res Perspect. 2018 24; 6 (5): e00432). In this assay, cells express cAMP sensor YFP-Epac-RLuc (Camyel) and GPR158. Cells are incubated with RLuc (Renilla luciferase) substrate coelenterazine H. Cells are treated with antibody and BRET ratio determined by calculating the ratio of the light emitted by RLuc (475 nm with a 30 nm band path width) over the light emitted by the Venus (535 nm with a 30 nm band path width) in order to measure an increase of the signal when cAMP increases. BRET ratio in cells treated with an antibody is compared to BRET ratio in cells not treated with the antibody. A higher BRET ratio in cells treated with an antibody compared to control cells not treated with the antibody indicates that the antibody increases CAMP production.

Another exemplary cell-based assay measures the effects of an antibody on cAMP levels using a fluorescence-based cAMP sensor cADDIS (Montana molecular) which involves Bacmam virus-based transduction of sensor into the cells.

In an exemplary cADDIs assay, HEK293T/17 cells are co-transfected with plasmids for GPR158, RGS7 and Gβ5 (150 μg/ml, GPR158: RGS7: Gβ5:4:1:1) by electroporation (Maxcyte). After 18-20 hours of recovery period post transfection, the cells are infected with the cADDIS CAMP sensor using the BacMam virus and seeded in a black flat bottom 96 well plate at a density of 50,000 cells per well. After 24 hours, the culture media is removed, and cells are washed with 1×PBS (containing CaCl₂) and MgCl₂) followed by incubation at room temperature for 15 minutes. Before antibody treatment, baseline fluorescence intensity is recorded using a fluorescence intensity module (FI Ex: 485 nm, Em: 520 nm) in Pherastar FSX microplate reader (BMG Labtech) followed by forskolin treatment for 15 min. Subsequently, the cells are treated with antibody and fluorescence intensity is recorded. The signal after 15 min of antibody treatment is normalized with respect to the buffer treated cells and the data are plotted in GraphPad Prism 10 software.

The cADDIS assay for Gai results in increase in fluorescence when CAMP is decreasing in the cells after treatment with antibody.

Another exemplary cell-based assay measures the effects of an antibody on cAMP levels using a Homogeneous Time-Resolved Fluorescence (HTRF) based cAMP Gai assay (cisbio).

In an exemplary HTRF Gai CAMP assay, HEK293T/17 cells are transfected with GPR158, RGS7 and Gβ5 plasmids, as described for cADDIs assay above, by Maxcyte electroporation. 24 hours post transfection, cells are harvested by centrifugation, counted, and seeded in a white 384 well plate at a density of 6000 cells per well after resuspending in the stimulation buffer provided with the kit. Afterwards, the cells are treated with an antibody or buffer for 30 minutes, followed by an additional incubation of 30 minutes at 37° C. after forskolin treatment. Subsequently, detection reagents, cAMP-Cryptate (donor) and Anti-cAMP d2 antibody (acceptor) are added and the plate was incubated at room temperature for 1 hour. The HTRF signal is measured using the HTRF optic module (donor excitation: 337 nm, donor emission: 620 nm, acceptor emission 665 nm) and laser energy source in the Pherastar FSX microplate reader (BMG Labtech).

The data are plotted as the ratio of fluorescence emission intensities by acceptor and donor (Em 665 nm/Em 620 nm). A CAMP standard curve is performed alongside to test the assay window and dynamic range. The HTRF ratios are multiplied by 10⁴ and the result is analyzed using GraphPad Prism 10 software.

HTRF assay takes advantage of the competition between Cryptate-labelled cAMP (donor) and unlabeled cAMP produced by the cells for binding to a d2-fluorophore labeled anti-CAMP antibody (acceptor). As competition with the unlabeled CAMP results in a reduction in FRET exchange, hence, HTRF signal is inversely proportional to the concentration of CAMP in the sample.

Another exemplary cell-based assay measures the effects of an antibody on GAP activity of RGS7-Gβ5 in cells expressing GPR158 and RGS7-Gβ5 using Bioluminescence Resonance Energy Transfer (BRET) between a recombinant Gβγ fused to a BRET acceptor and a recombinant Gβγ scavenger fused to a BRET donor (reporter), for example as in FIG. 4B. In some assays the BRET-acceptor-GB and BRET-acceptor-Gγ are encoded by separate constructs. In this assay, active G protein generates the BRET signal by virtue of free BRET-acceptor-Gβγ subunits interacting with the Gβγ scavenger-BRET donor (reporter). This signal is quenched when Ga is deactivated and recombines with BRET-acceptor-Gβγ to form inactive heterotrimer. In some cell-based screening methods, the cell expresses a recombinant target GPCR, a recombinant Gα_{oA}, a recombinant Gβγ scavenger fused to a BRET donor (reporter), a recombinant Gβγ fused to a BRET acceptor, a recombinant RGS7/Gβ5 (a GTPase Activation Protein (GAP) for the Gαi/o proteins), and a recombinant GPR158.

In some methods, the cell is treated with the antibody, and then, in order to activate (generate active Gao) and deactivate (form inactive heterotrimer), respectively, the recombinant target GPCR, the cell is exposed to an agonist of the recombinant target GPCR and then an antagonist of the recombinant target GPCR sequentially while dual-luminescence is measured at 475±30 nm and 535±30 nm. In some methods, dual luminescence is measured before and/or during and/or after the cell is exposed to the agonist and/or antagonist of the recombinant target GPCR. In some methods, dual luminescence is measured up to 60 seconds after exposing the cell to the antagonist of the recombinant target GPCR. A test cell is incubated with the substrate of the BRET donor in the presence of antibody and luminescence of the test cell is measured at 475±30 nm (light emitted by the BRET donor) and at 535±30 nm (light emitted by the BRET acceptor). The ratio of the light emitted by the BRET acceptor (535 nm with a 30 nm band path width) over the light emitted by the BRET donor (475 nm with a 30 nm band path width) is the test BRET ratio. In some methods, luminescence of the test cell at 475±30 nm and at 535±30 nm is measured, and the test BRET ratio determined, after treating the test cell with the antagonist.

The test BRET ratio can be compared to a ratio measured in a control cell incubated with the substrate of the BRET donor in the absence of antibody (the control BRET ratio). In some methods, the control cell is incubated with a buffer. A test BRET ratio greater than the control BRET ratio indicates that the antibody inhibits GAP activity of RGS7-Gß5 modulated by GPR158. Some methods include a step of determining a ratio in the test cell before incubation with the agonist of the recombinant target GPCR (test basal BRET ratio) and a step of subtracting the test basal BRET ratio from the test BRET ratio to determine a difference ratio (test ΔnetBRETratio). Some methods include a step of normalizing the test ΔnetBRETratio against a maximal ratio determined in the test cell after treatment with an agonist of the recombinant target GPCR (the test maximal netBRET ratio). Some methods include a step of determining a ratio in a control cell before incubation with the agonist of the recombinant target GPCR (control basal BRET ratio) and a step of subtracting the control basal BRET ratio from the control BRET ratio to determine a difference ratio (control ΔnetBRETratio). Some methods include a step of normalizing the control ΔnetBRETratio against a maximal ratio determined in the control cell after treatment with an agonist of the recombinant target GPCR (the control maximal netBRET ratio). In some methods, the test basal BRET ratio is compared to the control basal BRET ratio. The normalized test BRET ratio can be compared to a normalized BRET ratio measured in a control cell incubated with the substrate of the BRET donor in the absence of antibody (normalized control BRET ratio). A normalized test BRET ratio greater than the normalized control BRET ratio indicates that the antibody inhibits GAP activity of RGS7-Gβ5 modulated by GPR158.

In an exemplary BRET assay, as in FIG. 4B the recombinant target GPCR is dopamine D2 receptor, the recombinant Gβγ scavenger fused to a BRET donor is masGRK3ct-Nluc, the recombinant Gβγ fused to a BRET acceptor comprises Venus-156-239-Gß1 and Venus-1-155-Gγ2 (1), and the substrate for the BRET donor is furimazine. In an exemplary BRET assay, the recombinant target GPCR is dopamine D2 receptor, the agonist of the recombinant target GPCR is dopamine and the antagonist of the recombinant target GPCR is haloperidol.

In other embodiments, other GPCRs that activate Gai/o (target GPCRs), and their agonists and antagonists, other Gβγ scavengers, other BRET donors, other BRET substrates, and/or other BRET acceptors can be used in a cell-based screening assay to measure the effects of an antibody on GAP activity of RGS7-Gβ5 in cells expressing GPR158 and RGS7-Gβ5 using Bioluminescence Resonance Energy Transfer (BRET). Other exemplary FRET and BRET biosensors are as described, for example, in Kim, H. et al, (2022) Front. Cell Dev. Biol. 10:1007893.

Table 2 lists exemplary Gai/o coupled GPCRs (target GPCRs), along with their agonist and antagonist, that can be used to activate Gai/o proteins in a screening assays for antibodies of the invention. Column 1 lists receptor family, column 2 lists receptor name, column 3 lists agonist, and column 4 lists antagonist.

TABLE 2
Exemplary Gαi/o coupled Target GPCRs, agonists, and antagonists

| Receptor Family | Receptor Name | Agonist | Antagonist |
|---|---|---|---|
| Acetylcholine receptors | M2 | acetylcholine | Atropine |
| | M4 | | |
| Adenosine receptors | ADORA1 | Adenosine | Istradefylline |
| Adreno receptors | ADRA2A | adrenaline | Atropine |
| | ADRA2B | | |
| | ADRA2C | | |
| Angiotensin receptors | AGTR1 | Angiotensin II | Valsartan |
| Cannabinoid receptors | CNR1 | Anandamide | Ibipinabant |
| | CNR2 | | |
| Dopamine receptors | DRD2 | dopamine | Haloperidol |
| | DRD3 | | |
| | DRD4 | | |
| Leukotriene receptors | LTB4R | Leukotriene B4 | etalocib |
| | LTB4R2 | | LY255283 |
| Metabotropic GABA | GABBR2 | GABA | NA |
| receptors | | | |
| Metabotropic glutamate | GRM2 | Glutamate | LY341495 |
| receptors | | | |
| | GRM3 | | |
| | GRM4 | | |
| Oxytocin receptors | OXTR | Oxytocin | Retosiban |
| Opioid receptors | DOR | b-endorphin | Naltrexone |
| | KOR | dynorphin A | |
| | MOR | b-endorphin | |
| | OPRL1 | Nociceptin | |
| Purinoceptors | P2RY12 | ADP | ticagrelor |
| | P2RY13 | | cangrelor |
| | P2RY14 | UDP-Glucose | PPTN |
| Serotonin receptors | HTR1A | serotonin | Yohimbine |
| | HTR1B | | |
| | HTR1D | | |
| | HTR1E | | |
| | HTR1F | | |
| | HTR5A | | |
| Somatostatin receptors | SSTR1 | somatostatin-14 | BIM23454 |
| | SSTR2 | | |
| | SSTR3 | | |
| | SSTR4 | | |
| | SSTR5 | somatostatin-28 | |

Table 3 lists exemplary GPCRs that can be used to activate Go protein (target GPCRs) in a cell-based screening assays for antibodies of the invention. Column 1 lists receptor class, column 2 lists ligand type, column 3 lists receptor family, column 4 lists receptor name (UniProt), and column 5 lists receptor name according to Guide to Pharmacology (IUPHAR), GtP, guidetopharmacology.org).

TABLE 3
Exemplary Target GPCRs that can be used to activate Go

| | | | Receptor name | Receptor name |
|---|---|---|---|---|
| Class | Ligand type | Receptor family | (UniProt) | (GtP) |
| A | Aminergic | 5-Hydroxytryptamine | 5HT5A | 5-HT5A |
| A | Aminergic | 5-Hydroxytryptamine | 5HT2C | 5-HT2C |
| A | Aminergic | 5-Hydroxytryptamine | 5HT2B | 5-HT2B |
| A | Aminergic | 5-Hydroxytryptamine | 5HT2A | 5-HT2A |
| A | Aminergic | 5-Hydroxytryptamine | 5HT1F | 5-HTIF |
| A | Aminergic | 5-Hydroxytryptamine | 5HT1E | 5-HT1E |
| A | Aminergic | 5-Hydroxytryptamine | 5HT1D | 5-HT1D |
| A | Aminergic | 5-Hydroxytryptamine | 5HT1B | 5-HT1B |
| A | Aminergic | 5-Hydroxytryptamine | 5HT1A | 5-HT1A |
| A | Orphan | A orphans | OGR1 | GPR68 |
| A | Orphan | A orphans | MRGX2 | MRGPRX2 |
| A | Orphan | A orphans | MRGRD | MRGPRD |
| A | Orphan | A orphans | MAS | MAS1 |
| A | Orphan | A orphans | GPR84 | GPR84 |
| A | Orphan | A orphans | GPR6 | GPR6 |
| A | Orphan | A orphans | GPR4 | GPR4 |
| A | Orphan | A orphans | GPR37 | GPR37 |
| A | Orphan | A orphans | GPR34 | GPR34 |
| A | Orphan | A orphans | GPR33 | GPR33 |
| A | Orphan | A orphans | GPR31 | GPR31 |
| A | Orphan | A orphans | GPR22 | GPR22 |
| A | Orphan | A orphans | GPR20 | GPR20 |
| A | Orphan | A orphans | GPR17 | GPR17 |
| A | Orphan | A orphans | GPR12 | GPR12 |
| A | Orphan | A orphans | GP183 | GPR183 |
| A | Orphan | A orphans | G37L1 | GPR37L1 |
| A | Aminergic | Acetylcholine (muscarinic) | ACM4 | M4 |
| A | Aminergic | Acetylcholine (muscarinic) | ACM3 | M3 |
| A | Aminergic | Acetylcholine (muscarinic) | ACM2 | M2 |
| A | Aminergic | Acetylcholine (muscarinic) | ACM1 | M1 |
| A | Nucleotide | Adenosine | AA3R | A3 |
| A | Nucleotide | Adenosine | AAIR | A1 |
| A | Aminergic | Adrenoceptors | ADRB3 | β3- |
| | | | | adrenoceptor |
| A | Aminergic | Adrenoceptors | ADRB2 | β2- |
| | | | | adrenoceptor |
| A | Aminergic | Adrenoceptors | ADRB1 | β1- |
| | | | | adrenoceptor |
| A | Aminergic | Adrenoceptors | ADA2C | α2C- |
| | | | | adrenoceptor |
| A | Aminergic | Adrenoceptors | ADA2B | α2B- |
| | | | | adrenoceptor |
| A | Aminergic | Adrenoceptors | ADA2A | α2A- |
| | | | | adrenoceptor |
| A | Aminergic | Adrenoceptors | ADA1A | α1A- |
| | | | | adrenoceptor |
| A | Peptide | Angiotensin | AGTR2 | AT2 |
| A | Peptide | Angiotensin | AGTR1 | AT1 |
| A | Peptide | Apelin | APJ | apelin |
| A | Peptide | Bradykinin | BKRB2 | B2 |
| A | Peptide | Bradykinin | BKRB1 | B1 |
| A | Lipid | Cannabinoid | CNR2 | CB2 |
| A | Lipid | Cannabinoid | CNR1 | CB1 |
| A | Protein | Chemerin receptor | CML1 | chemerin |
| A | Protein | Chemokine | XCR1 | XCR1 |
| A | Protein | Chemokine | CXCR6 | CXCR6 |
| A | Protein | Chemokine | CXCR5 | CXCR5 |
| A | Protein | Chemokine | CXCR4 | CXCR4 |
| A | Protein | Chemokine | CXCR3 | CXCR3 |
| A | Protein | Chemokine | CXCR2 | CXCR2 |
| A | Protein | Chemokine | CXCR1 | CXCR1 |
| A | Protein | Chemokine | CX3C1 | CX3CR1 |
| A | Protein | Chemokine | CCR9 | CCR9 |
| A | Protein | Chemokine | CCR8 | CCR8 |
| A | Protein | Chemokine | CCR7 | CCR7 |
| A | Protein | Chemokine | CCR6 | CCR6 |
| A | Protein | Chemokine | CCR5 | CCR5 |
| A | Protein | Chemokine | CCR4 | CCR4 |
| A | Protein | Chemokine | CCR3 | CCR3 |
| A | Protein | Chemokine | CCR2 | CCR2 |
| A | Protein | Chemokine | CCR10 | CCR10 |
| A | Protein | Chemokine | CCR1 | CCR1 |
| A | Peptide | Cholecystokinin | CCKAR | CCK1 |
| A | Peptide | Complement peptide | C5AR1 | C5a1 |
| A | Aminergic | Dopamine | DRD5 | D5 |
| A | Aminergic | Dopamine | DRD4 | D4 |
| A | Aminergic | Dopamine | DRD3 | D3 |
| A | Aminergic | Dopamine | DRD2 | D2 |
| A | Aminergic | Dopamine | DRD1 | D1 |
| A | Peptide | Endothelin | EDNRB | ETB |
| A | Peptide | Endothelin | EDNRA | ETA |
| A | Steroid | Estrogen (G protein-coupled) | GPER1 | GPER |
| A | Peptide | Formylpeptide | FPR3 | FPR3 |
| A | Peptide | Formylpeptide | FPR2 | FPR2/ALX |
| A | Peptide | Formylpeptide | FPR1 | FPR1 |
| A | Lipid | Free fatty acid | FFAR4 | FFA4 |
| A | Lipid | Free fatty acid | FFAR3 | FFA3 |
| A | Lipid | Free fatty acid | FFAR2 | FFA2 |
| A | Lipid | Free fatty acid | FFAR1 | FFA1 |
| A | Peptide | Galanin | GALR3 | GAL3 |
| A | Peptide | Galanin | GALR2 | GAL2 |
| A | Peptide | Galanin | GALR1 | GAL1 |
| A | Peptide | Ghrelin | GHSR | ghrelin |
| A | Protein | Glycoprotein hormone | FSHR | FSH |
| A | Peptide | Gonadotrophin-releasing | GNRHR | GnRH1 |
| | | hormone | | |
| A | Lipid | GPR18, GPR55 and GPR119 | GPR18 | GPR18 |
| A | Aminergic | Histamine | HRH4 | H4 |
| A | Aminergic | Histamine | HRH3 | H3 |
| A | Aminergic | Histamine | HRH2 | H2 |
| A | Aminergic | Histamine | HRH1 | H1 |
| A | Alicarboxylic | Hydroxycarboxylic acid | HCAR3 | HCA3 |
| | acid | | | |
| A | Alicarboxylic | Hydroxycarboxylic acid | HCAR2 | HCA2 |
| | acid | | | |
| A | Alicarboxylic | Hydroxycarboxylic acid | HCAR1 | HCA1 |
| | acid | | | |
| A | Lipid | Leukotriene | OXER1 | OXE |
| A | Lipid | Leukotriene | LT4R2 | BLT2 |
| A | Lipid | Leukotriene | LT4R1 | BLT1 |
| A | Lipid | Leukotriene | CLTR2 | CysLT2 |
| A | Lipid | Leukotriene | CLTR1 | CysLT1 |
| A | Lipid | Lysophospholipid (LPA) | LPAR6 | LPA6 |
| A | Lipid | Lysophospholipid (LPA) | LPAR4 | LPA4 |
| A | Lipid | Lysophospholipid (LPA) | LPAR3 | LPA3 |
| A | Lipid | Lysophospholipid (LPA) | LPAR2 | LPA2 |
| A | Lipid | Lysophospholipid (LPA) | LPAR1 | LPA1 |
| A | Lipid | Lysophospholipid (S1P) | S1PR5 | S1P5 |
| A | Lipid | Lysophospholipid (S1P) | S1PR4 | S1P4 |
| A | Lipid | Lysophospholipid (S1P) | S1PR3 | S1P3 |
| A | Lipid | Lysophospholipid (S1P) | S1PR1 | S1P1 |
| A | Peptide | Melanin-concentrating | MCHR1 | MCH1 |
| | | hormone | | |
| A | Peptide | Melanocortin | MC4R | MC4 |
| A | Peptide | Melanocortin | MC3R | MC3 |
| A | Melatonin | Melatonin | MTR1B | MT2 |
| A | Melatonin | Melatonin | MTR1A | MT1 |
| A | Peptide | Neuropeptide FF/neuropeptide | NPFF2 | NPFF2 |
| | | AF | | |
| A | Peptide | Neuropeptide FF/neuropeptide | NPFF1 | NPFF1 |
| | | AF | | |
| A | Peptide | Neuropeptide W/neuropeptide | NPBW2 | NPBW2 |
| | | B | | |
| A | Peptide | Neuropeptide W/neuropeptide | NPBW1 | NPBW1 |
| | | B | | |
| A | Peptide | Neuropeptide Y | NPY5R | Y5 |
| A | Peptide | Neuropeptide Y | NPY4R | Y4 |
| A | Peptide | Neuropeptide Y | NPY2R | Y2 |
| A | Peptide | Neuropeptide Y | NPY1R | Y1 |
| A | Peptide | Neurotensin | NTR1 | NTS1 |
| A | Peptide | Opioid | OPRX | NOP |
| A | Peptide | Opioid | OPRM | μ |
| A | Peptide | Opioid | OPRK | κ |
| A | Peptide | Opioid | OPRD | δ |
| A | Sensory | Opsins | OPN5 | OPN5 |
| A | Sensory | Opsins | OPN4 | OPN4 |
| A | Peptide | Orexin | OX2R | OX2 |
| A | Peptide | Orexin | OX1R | OX1 |
| A | Nucleotide | P2Y | P2Y14 | P2Y14 |
| A | Nucleotide | P2Y | P2Y13 | P2Y13 |
| A | Nucleotide | P2Y | P2RY2 | P2Y2 |
| A | Nucleotide | P2Y | P2RY1 | P2Y1 |
| A | Peptide | Peptide P518 | QRFPR | QRFP |
| A | Lipid | Platelet-activating factor | PTAFR | PAF |
| A | Protein | Prokineticin | PKR2 | PKR2 |
| A | Lipid | Prostanoid | PI2R | IP |
| A | Lipid | Prostanoid | PF2R | FP |
| A | Lipid | Prostanoid | PE2R4 | EP4 |
| A | Lipid | Prostanoid | PE2R3 | EP3 |
| A | Lipid | Prostanoid | PE2R1 | EP1 |
| A | Lipid | Prostanoid | PD2R2 | DP2 |
| A | Peptide | Proteinase-activated | PAR2 | PAR2 |
| A | Peptide | Proteinase-activated | PAR1 | PAR1 |
| A | Peptide | Relaxin family peptide | RXFP2 | RXFP2 |
| A | Peptide | Relaxin family peptide | RXFP1 | RXFP1 |
| A | Peptide | Relaxin family peptide | RL3R2 | RXFP4 |
| A | Peptide | Relaxin family peptide | RL3R1 | RXFP3 |
| A | Peptide | Somatostatin | SSR5 | SST5 |
| A | Peptide | Somatostatin | SSR4 | SST4 |
| A | Peptide | Somatostatin | SSR3 | SST3 |
| A | Peptide | Somatostatin | SSR2 | SST2 |
| A | Peptide | Somatostatin | SSR1 | SST1 |
| A | Alicarboxylic | Succinate | SUCR1 | succinate |
| | acid | | | |
| A | Peptide | Vasopressin and oxytocin | VIAR | VIA |
| A | Peptide | Vasopressin and oxytocin | OXYR | OT |
| B1 | Peptide | VIP and PACAP | VIPR1 | VPAC1 |
| B2 | Adhesion | ADGRG | AGRG3 | ADGRG3 |
| C | Ion | Calcium-sensing | CASR | CaS |
| C | Amino acid | Metabotropic glutamate | GRM2 | mGlu2 |
| C | Amino acid | Metabotropic glutamate | GRM4 | mGlu4 |
| C | Amino acid | Metabotropic glutamate | GRM5 | mGlu5 |
| C | Amino acid | Metabotropic glutamate | GRM6 | mGlu6 |
| C | Amino acid | Metabotropic glutamate | GRM8 | mGlu8 |
| C | Amino acid | Metabotropic glutamate | GRM3 | mGlu3 |
| C | Amino acid | Metabotropic glutamate | GRM1 | mGlu1 |
| C | Amino acid | Metabotropic glutamate | GRM7 | mGlu7 |
| F | Protein | Frizzled | FZD2 | FZD2 |
| F | Protein | Frizzled | FZD7 | FZD7 |
| F | Protein | Frizzled | SMO | SMO |
| F | Protein | Frizzled | FZD1 | FZD1 |
| F | Protein | Frizzled | FZD6 | FZD6 |
| F | Protein | Frizzled | FZD9 | FZD9 |

Table 4 shows exemplary BRET donors, substrates, and Table 5 shows exemplary BRET acceptors, that can be used in screening assays for antibodies of the invention.

TABLE 4
Exemplary BRET donors and substrates

| BRET donor | |
|---|---|
| λem = 480 + 20 nm (Glow type | |
| luminescence) | Substrate |
| GLuc | Coelenterazine |
| | (h) |
| RLuc | Coelenterazine |
| | (h) |
| NanoLuc (Nluc) | furimazine |

TABLE 5
Exemplary BRET Acceptors
BRET acceptor
λexc = 480 + 20 nm
λem = 540 + 30 nm
(Fluorescence)

Venus
GFP
YFP
eGFP

Other exemplary FRET and BRET biosensors useful in screening assays for antibodies of the invention are shown in FIG. 22 (see Kim, H. et al, (2022) Front. Cell Dev. Biol. 10:1007893). Column 1 shows detection step, column 2 shows target GPCR, column 3 shows ligand used, column 4 shows detection method, column 5 shows FRET or BRET pair, column 6 shows cell lines used, column 7 shows notes, and column 8 shows reference.

### FIG. 22 REFERENCES

Borroto-Escuela, D. O., Romero-Fernandez, W., Tarakanov, A. O., Marcellino, D., Ciruela, F., Agnati, L. F., et al. (2010). Dopamine D2 and 5-hydroxytryptamine 5-HT(₂A) receptors assemble into functionally interacting heteromers. Biochem. Biophys. Res. Commun. 401 (4), 605-610
Charest, P. G., Terrillon, S., and Bouvier, M. (2005). Monitoring agonist-promoted conformational changes of beta-arrestin in living cells by intramolecular BRET. EMBO Rep. 6 (4), 334-340.
Erdogmus, S., Storch, U., Danner, L., Becker, J., Winter, M., Ziegler, N., et al. (2019). Helix 8 is the essential structural motif of mechanosensitive GPCRs. Nat. Commun. 10 (1), 5784.
Gales, C., Rebois, R. V., Hogue, M., Trieu, P., Breit, A., Hebert, T. E., et al. (2005). Real-time monitoring of receptor and G-protein interactions in living cells. Nat. Methods 2 (3), 177-184.
Gupte, T. M., Malik, R. U., Sommese, R. F., Ritt, M., and Sivaramakrishnan, S. (2017). Priming GPCR signaling through the synergistic effect of two G proteins. Proc. Natl. Acad. Sci. U.S.A. 114 (14), 3756-3761.
Hires, S. A., Zhu, Y., and Tsien, R. Y. (2008). Optical measurement of synaptic glutamate spillover and reuptake by linker optimized glutamate-sensitive fluorescent reporters. Proc. Natl. Acad. Sci. U.S.A. 105 (11), 4411-4416.
Hlavackova, V., Zabel, U., Frankova, D., Batz, J., Hoffmann, C., Prezeau, L., et al. (2012). Sequential inter- and intrasubunit rearrangements during activation of dimeric metabotropic glutamate receptor 1. Sci. Signal. 5 (237), ra59.
Hoffmann, C., Gaietta, G., Bunemann, M., Adams, S. R., Oberdorff-Maass, S., Behr, B., et al. (2005). A FLASH-based FRET approach to determine G protein-coupled receptor activation in living cells. Nat. Methods2 (3), 171-176.
Lee, M. H., Appleton, K. M., Strungs, E. G., Kwon, J. Y., Morinelli, T. A., Peterson, Y. K., et al. (2016). The conformational signature of beta-arrestin2 predicts its trafficking and signalling functions. Nature 531 (7596), 665-668.
Lukasiewicz, S., Polit, A., Kedracka-Krok, S., Wedzony, K., Mackowiak, M., and Dziedzicka-Wasylewska, M. (2010). Hetero-dimerization of serotonin 5-HT (2A) and dopamine D (2) receptors. Biochim. Biophys. Acta1803 (12), 1347-1358.
Maier-Peuschel, M., Frolich, N., Dees, C., Hommers, L. G., Hoffmann, C., Nikolaev, V. O., et al. (2010). A Gai/o uorescence resonance energy transfer-based M2 muscarinic receptor sensor reveals rapid kinetics of allosteric modulation. J. Biol. Chem. 285 (12), 8793-8800.
Malik, R. U., Ritt, M., DeVree, B. T., Neubig, R. R., Sunahara, R. K., and Sivaramakrishnan, S. (2013). Detection of G protein-selective G protein-coupled receptor (GPCR) conformations in live cells. J. Biol. Chem. 288 (24), 17167-17178.
Manchanda, Y., Ramchunder, Z., Shchepinova, M. M., Rutter, G. A., Inoue, A., Tate, E. W., et al. (2021). Expression of mini-G proteins specifically halt cognate GPCR trafficking and intracellular signalling. bioRxiv, 2021. doi: 10.1101/2021.11.24.469908
Maziarz, M., Park, J. C., Leyme, A., Marivin, A., Garcia-Lopez, A., Patel, P. P., et al. (2020).
Nakanishi, J., Takarada, T., Yunoki, S., Kikuchi, Y., and Maeda, M. (2006). FRET-based monitoring of conformational change of the beta2 adrenergic receptor in living cells. Biochem. Biophys. Res. Commun. 343 (4), 1191-1196.
Nikolaev, V. O., Hoffmann, C., Bunemann, M., Lohse, M. J., and Vilardaga, J. P. (2006). Molecular basis of partial agonism at the neurotransmitter alpha2A-adrenergic receptor and Gi-protein heterotrimer. J. Biol. Chem. 281 (34), 24506-24511.
Nuber, S., Zabel, U., Lorenz, K., Nuber, A., Milligan, G., Tobin, A. B., et al. (2016). β-Arrestin biosensors reveal a rapid, receptor-dependent activation/deactivation cycle. Nature 531 (7596), 661-664.
Oishi, A., Dam, J., and Jockers, R. (2020). β-Arrestin-2 BRET biosensors detect different β-arrestin-2 conformations in interaction with GPCRs. ACSSens. 5 (1), 57-64.
Reiner, S., Ambrosio, M., Hoffmann, C., and Lohse, M. J. (2010). Differential signaling of the endogenous agonists at the beta2-adrenergic receptor. J. Biol. Chem. 285 (46), 36188-36198.
Rochais, F., Vilardaga, J. P., Nikolaev, V. O., Bunemann, M., Lohse, M. J., and Engelhardt, S. (2007). Real-time optical recording of beta1-adrenergic receptor activation reveals supersensitivity of the Arg389 variant to carvedilol. J. Clin. Invest. 117 (1), 229-235.
Szalai, B., Barkai, L., Turu, G., Szidonya, L., Varnai, P., and Hunyady, L. (2012). Allosteric interactions within the AT₁ angiotensin receptor homodimer: Role of the conserved DRY motif. Biochem. Pharmacol. 84 (4), 477-485.
Tsien, R. Y. (2006). Breeding and building molecules to spy on cells and tumors. Keio J. Med. 55 (4), 127-140.
Vilardaga, J. P., Bunemann, M., Krasel, C., Castro, M., and Lohse, M. J. (2003). Measurement of the millisecond activation switch of G protein-coupled receptors in living cells. Nat. Biotechnol. 21 (7), 807-812.
Vilardaga, J. P., Steinmeyer, R., Harms, G. S., and Lohse, M. J. (2005). Molecular basis of inverse agonism in a G protein-coupled receptor. Nat. Chem. Biol. 1 (1), 25-28.
Wan, Q., Okashah, N., Inoue, A., Nehme, R., Carpenter, B., Tate, C. G., et al. (2018). Mini G protein probes for active G protein-coupled receptors (GPCRs) in live cells. J. Biol. Chem. 293 (19), 7466-7473.
Zhang, X., Tan, F., and Skidgel, R. A. (2013). Carboxypeptidase M is a positive allosteric modulator of the kinin B1 receptor. J. Biol. Chem. 288 (46), 33226-33240.
Zhang, Y. L., Frangos, J. A., and Chachisvilis, M. (2009). Mechanical stimulus alters conformation of type 1 parathyroid hormone receptor in bone cells. Am. J. Physiol. Cell Physiol. 296 (6), C1391-C1399.

### B. Animal Models

The activity of antibodies can be measured in ex vivo brain slices from GPR158 knock-out and wild-type mice, for example Gpr 158^{-/-} mice (Knockout Mouse Programme, (KOMP) (Gpr158^{tm1(KOMP)Vlcg}) maintained on a C57/β16 background and bred as heterozygous pairs to generate Gpr158^{-/-} and Gpr158^{+/+} littermates) Ex vivo brain slices treated with and without an antibody can be assessed by various criteria including increase in number of action potentials fired over the ramp while decreasing the amount of current necessary to elicit the first action potential without changes in the resting membrane potential.

Tests on an antibody are usually performed in conjunction with a control in which a parallel experiment is conducted except that the antibody is absent (e.g., replaced by vehicle). Reduction, delay or inhibition of signs or symptoms disease attributable to an antibody under test can then be assessed relative to the control.

### VI. Patients Amenable to Treatment

The present regimes can also be used in treatment or prophylaxis of an affective disorder, a mood disorder or a brain disorder and of an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder.

Patients amenable to treatment include individuals at risk of disease but not showing symptoms, as well as patients presently showing symptoms. Patients at risk of disease include those having a known genetic risk of disease. Such individuals include those having relatives who have experienced this disease, and those whose risk is determined by analysis of genetic or biochemical markers. Genetic markers of risk include mutations in GPR158, as well as mutations in other genes associated with an affective disorder, a mood disorder or a brain disorder and/or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder.

In asymptomatic patients, treatment can begin at any age (e.g., 10, 20, 30). Usually, however, it is not necessary to begin treatment until a patient reaches 40, 50, 60 or 70 years of age. Treatment typically entails multiple dosages over a period of time. Treatment can be monitored by assaying antibody levels over time. If the response falls, a booster dosage is indicated.

### VII. Nucleic Acids

The invention further provides a nucleic acid encoding any of the heavy chains described above (e.g., SEQ ID NO:2 or SEQ ID NO:13). Optionally, such nucleic acids further encode a signal peptide and can be expressed with the signal peptide linked to the variable region. Coding sequences of nucleic acids can be operably linked with regulatory sequences to ensure expression of the coding sequences, such as a promoter, enhancer, ribosome binding site, transcription termination signal, and the like. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

### VIII. Conjugated Antibodies

Conjugated antibodies that specifically bind to antigens, such as GPR158, are useful in detecting the presence of GPR158; monitoring and evaluating the efficacy of therapeutic agents being used to treat patients diagnosed with an affective disorder, a mood disorder or a brain disorder, or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder. For example, such antibodies can be conjugated with other therapeutic moieties, other proteins, other antibodies, and/or detectable labels. See WO 03/057838; U.S. Pat. No. 8,455,622. Such therapeutic moieties can be any agent that can be used to treat, combat, ameliorate, prevent, or improve an unwanted condition or disease in a patient, such as an affective disorder, a mood disorder or a brain disorder, or an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder.

Conjugated therapeutic moieties can include cytotoxic agents, cytostatic agents, neurotrophic agents, neuroprotective agents, radiotherapeutic agents, immunomodulators, or any biologically active agents that facilitate or enhance the activity of the antibody. A cytotoxic agent can be any agent that is toxic to a cell. A cytostatic agent can be any agent that inhibits cell proliferation. A neurotrophic agent can be any agent, including chemical or proteinaceous agents, that promotes neuron maintenance, growth, or differentiation. A neuroprotective agent can be agent, including chemical or proteinaceous agents, that protects neurons from acute insult or degenerative processes. An immunomodulator can be any agent that stimulates or inhibits the development or maintenance of an immunologic response. A radiotherapeutic agent can be any molecule or compound that emits radiation. If such therapeutic moieties are coupled to a GPR158-specific antibody, such as the antibodies described herein, the coupled therapeutic moieties will have a specific affinity for GPR158-expressing cells over cells not expressing GPR158. Consequently, administration of the conjugated antibodies directly targets GPR158-expressing cells with minimal damage to surrounding cells not expressing GPR158. This can be particularly useful for therapeutic moieties that are too toxic to be administered on their own. In addition, smaller quantities of the therapeutic moieties can be used.

Some such antibodies can be modified to act as immunotoxins. See, e.g., U.S. Pat. No. 5,194,594. For example, ricin, a cellular toxin derived from plants, can be coupled to antibodies by using the bifunctional reagents S-acetylmercaptosuccinic anhydride for the antibody and succinimidyl 3-(2-pyridyldithio) propionate for ricin. See Pietersz et al., Cancer Res. 48 (16): 4469-4476 (1998). The coupling results in loss of B-chain binding activity of ricin, while impairing neither the toxic potential of the A-chain of ricin nor the activity of the antibody. Similarly, saporin, an inhibitor of ribosomal assembly, can be coupled to antibodies via a disulfide bond between chemically inserted sulfhydryl groups. See Polito et al., Leukemia 18:1215-1222 (2004).

Some such antibodies can be linked to radioisotopes. Examples of radioisotopes include, for example, yttrium⁹⁰ (90Y), indium¹¹¹ (111In), ¹³¹I, ^{99mTc}, radiosilver-111, radiosilver-199, and Bismuth²¹³. Linkage of radioisotopes to antibodies may be performed with conventional bifunction chelates. For radiosilver-111 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1: S91-S95 (2001).

Some such antibodies can be linked to other therapeutic moieties. Such therapeutic moieties can be, for example, cytotoxic, cytostatic, neurotrophic, or neuroprotective. For example, antibodies can be conjugated with toxic chemotherapeutic drugs such as maytansine, geldanamycin, tubulin inhibitors such as tubulin binding agents (e.g., auristatins), or minor groove binding agents such as calicheamicin. Other representative therapeutic moieties include agents known to be useful for treatment, management, or amelioration of an affective disorder, a mood disorder or a brain disorder, or of an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder.

Antibodies can also be coupled with other proteins. For example, antibodies can be coupled with Fynomers. Fynomers are small binding proteins (e.g., 7 kDa) derived from the human Fyn SH3 domain. They can be stable and soluble, and they can lack cysteine residues and disulfide bonds. Fynomers can be engineered to bind to target molecules with the same affinity and specificity as antibodies. They are suitable for creating multi-specific fusion proteins based on antibodies. For example, Fynomers can be fused to N-terminal and/or C-terminal ends of antibodies to create bi- and tri-specific FynomAbs with different architectures. Fynomers can be selected using Fynomer libraries through screening technologies using FACS, Biacore, and cell-based assays that allow efficient selection of Fynomers with optimal properties. Examples of Fynomers are disclosed in Grabulovski et al., J. Biol. Chem. 282:3196-3204 (2007); Bertschinger et al., Protein Eng. Des. Sel. 20:57-68 (2007); Schlatter et al., MAbs. 4:497-508 (2011); Banner et al., Acta. Crystallogr. D. Biol. Crystallogr. 69 (Pt6): 1124-1137 (2013); and Brack et al., Mol. Cancer Ther. 13:2030-2039 (2014).

The antibodies disclosed herein can also be coupled or conjugated to one or more other antibodies (e.g., to form antibody heteroconjugates). Such other antibodies can bind to different epitopes within GPR158 or can bind to a different target antigen.

Antibodies can also be coupled with a detectable label. Such antibodies can be used, for example, for diagnosing an affective disorder, a mood disorder or a brain disorder, and/or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder, and/or for assessing efficacy of treatment. Such antibodies are particularly useful for performing such determinations in subjects having or being susceptible to an affective disorder, a mood disorder or a brain disorder, and/or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder, or in appropriate biological samples obtained from such subjects. Representative detectable labels that may be coupled or linked to an antibody include various enzymes, such as horseradish peroxidase, alkaline phosphatase, beta-galactosidase, or acetylcholinesterase; prosthetic groups, such streptavidin/biotin and avidin/biotin; fluorescent materials, such as umbelliferone, fluorescein, fluorescein isothiocyanate, rhodamine, dichlorotriazinylamine fluorescein, dansyl chloride or phycoerythrin; luminescent materials, such as luminol; bioluminescent materials, such as luciferase, luciferin, and aequorin; radioactive materials, such as radiosilver-111, radiosilver-199, Bismuth²¹³, iodine (¹³¹I, ¹²⁵I, ¹²³I, ¹²¹I), carbon (¹⁴C), sulfur (⁵S), tritium (³H), indium (¹¹⁵In, ¹¹³In, ¹¹²In, ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies; nonradioactive paramagnetic metal ions; and molecules that are radiolabelled or conjugated to specific radioisotopes.

Linkage of radioisotopes to antibodies may be performed with conventional bifunction chelates. For radiosilver-111 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1: S91-S95 (2001).

Therapeutic moieties, other proteins, other antibodies, and/or detectable labels may be coupled or conjugated, directly or indirectly through an intermediate (e.g., a linker), to an antibody of the invention. See e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985); and Thorpe et al., Immunol. Rev., 62:119-58 (1982). Suitable linkers include, for example, cleavable and non-cleavable linkers. Different linkers that release the coupled therapeutic moieties, proteins, antibodies, and/or detectable labels under acidic or reducing conditions, on exposure to specific proteases, or under other defined conditions can be employed.

### IX. Pharmaceutical Compositions and Methods of Use

In prophylactic applications, an antibody or agent for inducing an antibody or a pharmaceutical composition the same is administered to a patient susceptible to, or otherwise at risk of a disease (e.g., an affective disorder, a mood disorder or a brain disorder) in regime (dose, frequency and route of administration) effective to reduce the risk, lessen the severity, or delay the onset of at least one sign or symptom of the disease. In therapeutic applications, an antibody or agent to induce an antibody is administered to a patient suspected of, or already suffering from a disease (e.g., an affective disorder, a mood disorder or a brain disorder) in a regime (dose, frequency and route of administration) effective to ameliorate or at least inhibit further deterioration of at least one sign or symptom of the disease.

A regime is considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is human or an animal, other medications administered, and whether treatment is prophylactic or therapeutic.

Exemplary dosage ranges for antibodies are from about 0.01 to 60 mg/kg, or from about 0.1 to 3 mg/kg or 0.15-2 mg/kg or 0.15-1.5 mg/kg, of patient body weight. Antibody can be administered such doses daily, on alternative days, weekly, fortnightly, monthly, quarterly, or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months.

The amount of an agent for active administration varies from 0.1-500 μg per patient and more usually from 1-100 or 1-10 μg per injection for human administration. The timing of injections can vary significantly from once a day, to once a year, to once a decade. A typical regimen consists of an immunization followed by booster injections at time intervals, such as 6 week intervals or two months. Another regimen consists of an immunization followed by booster injections 1, 2 and 12 months later. Another regimen entails an injection every two months for life. Alternatively, booster injections can be on an irregular basis as indicated by monitoring of immune response.

Antibodies or agents for inducing antibodies are preferably administered via a peripheral route (i.e., one in which an administered or induced antibody crosses the blood brain barrier to reach an intended site in the brain. Routes of administration include topical, intravenous, oral, subcutaneous, intraarterial, intracranial, intrathecal, intraperitoneal, intranasal, intraocular, or intramuscular. Preferred routes for administration of antibodies are intravenous and subcutaneous. Preferred routes for active immunization are subcutaneous and intramuscular. This type of injection is most typically performed in the arm or leg muscles.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

The present regimes can be administered in combination with another agent effective in treatment or prophylaxis of the disease being treated. For example, in the case of an affective disorder, a mood disorder or a brain disorder, the present regimes can be combined administered in combination with, for example, a selective serotonin reuptake inhibitor (SSRI), a serotonin-norepinephrine reuptake inhibitor (SNRI), a serotonin modulator and stimulator (SMS), a serotonin antagonist and reuptake inhibitor (SARI), a norepinephrine-dopamine reuptake inhibitor (NDRI), a tricyclic antidepressant (TCA), a tetracyclic antidepressant (TeCA), a monoamine oxidase inhibitor (MAOI), or a NMDA receptor antagonist. Exemplary NMDA receptor antagonists are ketamine, esketamine or dextromethorphan.

Antibodies are administered in an effective regime meaning a dosage, route of administration and frequency of administration that delays the onset, reduces the severity, inhibits further deterioration, and/or ameliorates at least one sign or symptom of a disorder being treated. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an antibody are 0.1-60 mg/kg (e.g., 0.5, 3, 10, 30, or 60 mg/kg), or 0.5-5 mg/kg body weight (e.g., 0.5, 1, 2, 3, 4 or 5 mg/kg) or 10-4000 mg or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Some antibodies can be administered into the systemic circulation by intravenous or subcutaneous administration. Intravenous administration can be, for example, by infusion over a period such as 30-90 min.

The frequency of administration depends on the half-life of the antibody in the circulation, the condition of the patient and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

### X. Diagnostics and Monitoring Methods

Also provided are methods of detecting GPR158 in a subject, for example, by measuring GPR158 in a sample from a subject or by in vivo imaging of GPR158 in a subject. Such methods are useful to diagnose or confirm diagnosis of an affective disorder, a mood disorder or a brain disorder, or of an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder or susceptibility thereto. The methods can also be used on asymptomatic subjects. The methods are also useful for monitoring disease progression and/or response to treatment in subjects who have been previously diagnosed with an affective disorder, a mood disorder or a brain disorder, or of an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder.

The methods work by administering a reagent, such as any of the antibodies that binds GPR158 described in this application (e.g., a llama, humanized, chimeric or veneered Nb20 antibody), to the subject and then detecting the agent after it has bound. If desired, the clearing response can be avoided by using antibody fragments lacking a full length constant region, such as Fabs. In some methods, the same antibody can serve as both a treatment and diagnostic reagent.

Diagnostic reagents can be administered by intranasal or intravenous injection into the body of the patient, or directly into the brain by intracranial injection or by drilling a hole through the skull. The dosage of reagent should be within the same ranges as for treatment methods. Typically, the reagent is labeled, although in some methods, the primary reagent with affinity for GPR158 is unlabeled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using positron emission tomography (PET) or single-photon emission computed tomography (SPECT).

Biological samples obtained from a subject having, suspected of having, or at risk of having an affective disorder, a mood disorder or a brain disorder, or of an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder can be contacted with the antibodies disclosed herein to assess the presence of GPR158. For example, levels of GPR158 in such subjects may be compared to those present in healthy subjects. Alternatively, levels of GPR158 in such subjects receiving treatment for an affective disorder, a mood disorder or a brain disorder may be compared to those of subjects who have not been treated for an affective disorder, a mood disorder or a brain disorder, or of an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder. Some such tests involve a biopsy of tissue obtained from such subjects. ELISA assays may also be useful methods, for example, for assessing GPR158 in fluid samples.

### XI. Kits

The invention further provides kits (e.g., containers) comprising an antibody disclosed herein and related materials, such as instructions for use (e.g., package insert). The instructions for use may contain, for example, instructions for administration of the antibody and optionally one or more additional agents. The containers of antibody may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products

Kits can also include a second container comprising a pharmaceutically-acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

### XII. Other Applications

The antibodies can be used for detecting GPR158, or fragments thereof, in the context of clinical diagnosis or treatment or in research. For example, the antibodies can be used to detect the presence of GPR158 in a biological sample. Binding of the antibodies to the biological sample can be compared to binding of the antibodies to a control sample. The control sample and the biological sample can comprise cells of the same tissue origin. Control samples and biological samples can be obtained from the same individual or different individuals and on the same occasion or on different occasions. If desired, multiple biological samples and multiple control samples are evaluated on multiple occasions to protect against random variation independent of the differences between the samples. A direct comparison can then be made between the biological sample(s) and the control sample(s) to determine whether antibody binding (i.e., the presence of GPR158) to the biological sample(s) is increased, decreased, or the same relative to antibody binding to the control sample(s). Increased binding of the antibody to the biological sample(s) relative to the control sample(s) indicates the presence of GPR158 in the biological sample(s). In some instances, the increased antibody binding is statistically significant. Optionally, antibody binding to the biological sample is at least 1.5-fold, 2-fold, 3-fold, 4-fold, 5-fold, 10-fold, 20-fold, or 100-fold higher than antibody binding to the control sample.

In addition, the antibodies can be used to detect the presence of the GPR158 in a biological sample to monitor and evaluate the efficacy of a therapeutic agent being used to treat a patient diagnosed with an affective disorder, a mood disorder or a brain disorder, or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder. A biological sample from a patient diagnosed with an affective disorder, a mood disorder or a brain disorder, or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder is evaluated to establish a baseline for the binding of the antibodies to the sample (i.e., a baseline for the presence of the GPR158 in the sample) before commencing therapy with the therapeutic agent. In some instances, multiple biological samples from the patient are evaluated on multiple occasions to establish both a baseline and measure of random variation independent of treatment. A therapeutic agent is then administered in a regimen. The regimen may include multiple administrations of the agent over a period of time. Optionally, binding of the antibodies (i.e., presence of GPR158) is evaluated on multiple occasions in multiple biological samples from the patient, both to establish a measure of random variation and to show a trend in response to immunotherapy. The various assessments of antibody binding to the biological samples are then compared. If only two assessments are made, a direct comparison can be made between the two assessments to determine whether antibody binding (i.e., presence of GPR158) has increased, decreased, or remained the same between the two assessments. If more than two measurements are made, the measurements can be analyzed as a time course starting before treatment with the therapeutic agent and proceeding through the course of therapy. Assessment of antibody binding can be made in conjunction with assessing other signs and symptoms of with an affective disorder, a mood disorder or a brain disorder, or with an affective disorder, a mood disorder or a brain disorder associated with depression, disruptive mood dysregulation disorder, major depressive disorder (MDD), dysthymia, stress induced depression, a generalized mood disorder, chronic stress disorder, anhedonia, or an anxiety disorder.

The antibodies can also be used as research reagents for laboratory research in detecting GPR158, or fragments thereof. In such uses, antibodies can be labeled with fluorescent molecules, spin-labeled molecules, enzymes, or radioisotopes, and can be provided in the form of kit with all the necessary reagents to perform the detection assay. The antibodies can also be used to purify GPR158, or binding partners of GPR158, e.g., by affinity chromatography.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1: Development and Characterization of Nanobodies Targeting mGlyR

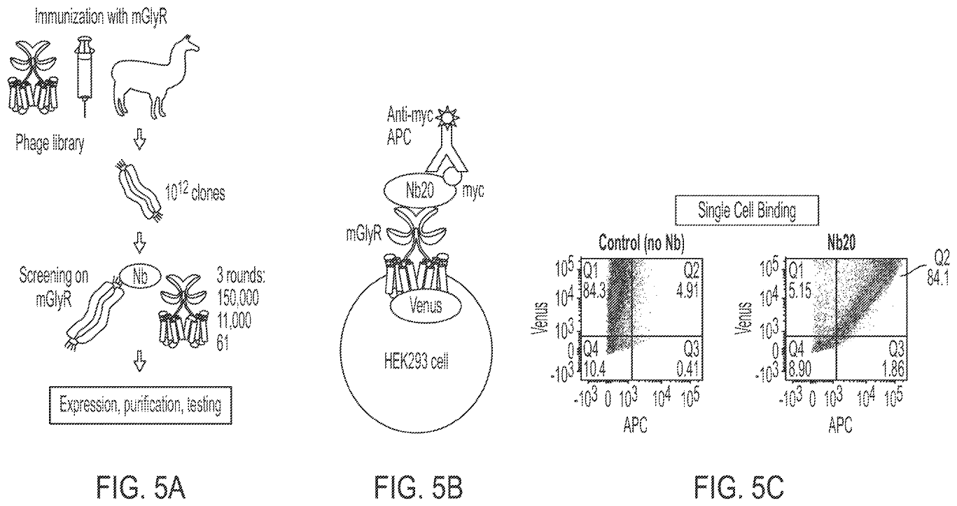

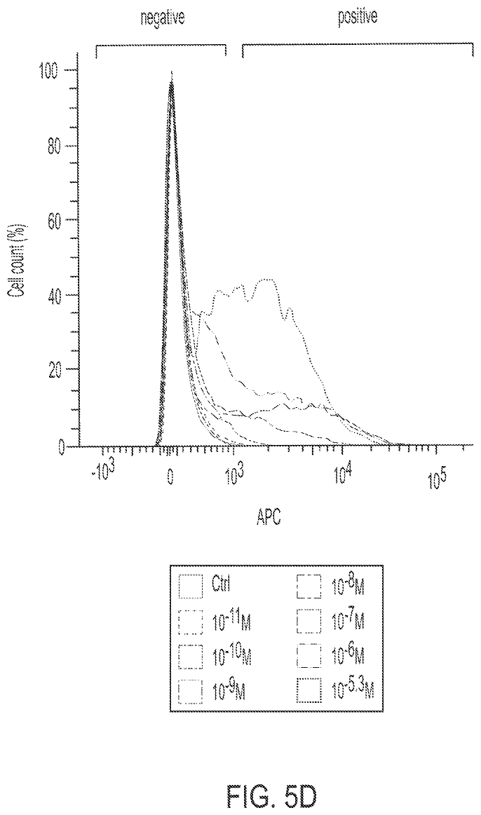

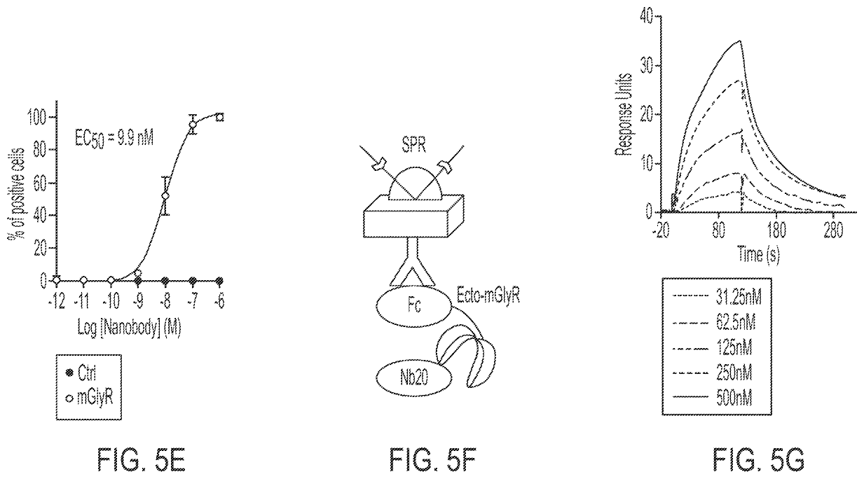

Given complete lack of selective chemical probes for mGlyR, the inventors sought to obtain small protein ligands to alter mGlyR activity. The presence of extensive extracellular elements in mGlyR, makes such a strategy attractive considering recent success with other class C GPCRs (51-52). The inventors chose to generate single-domain antibodies (nanobodies) for their high affinity towards targets and emerging potential for therapeutic translation. Phage library prepared from llamas immunized with recombinant GPR158 (mGlyR) was screened against HEK293 cell membranes containing mGlyR (FIG. 5A). After three rounds of enrichment and rescreening 61 individual clones were chosen. Corresponding nanobodies were isolated following expression in E. coli and tested for mGlyR binding using flow cytometry strategy (FIG. 5B). Three clones showed positive interaction with GPR158-expressing cells. Of these, clone number 20 (Nb20) showed the most robust signal and was chosen for further studies.

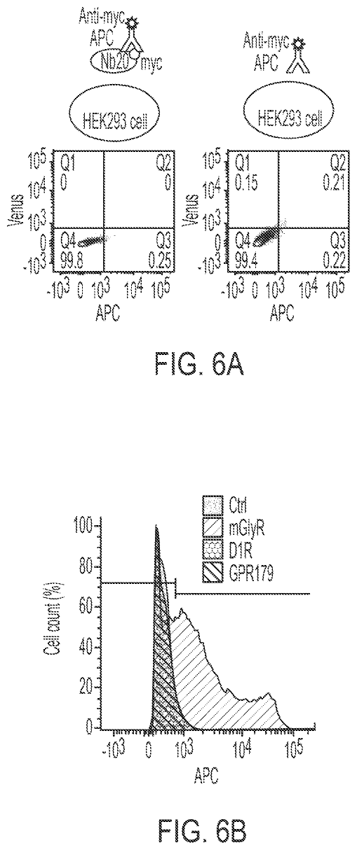

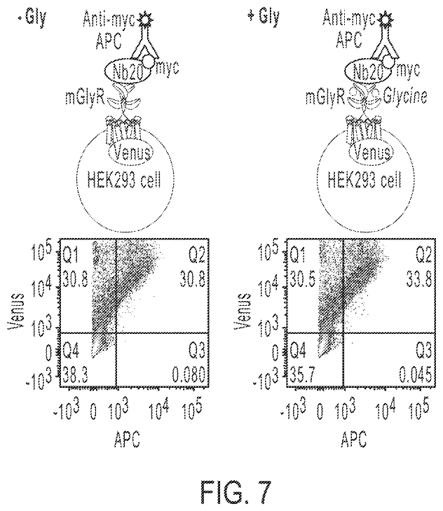

First, the inventors have used flow cytometry to characterize Nb20 binding to mGlyR. Cells transfected with Venus-tagged full length GPR158 were incubated with purified myc-tagged Nb20 and its interaction with cells was monitored by APC-conjugated antibodies against myc (FIG. 5B). Using this approach, the inventors were able to detect robust labeling of majority of GPR158-transfected cells with Nb20 when excess Nb20 was used in the assay (FIG. 5C). Control experiments with cells lacking GPR158, Nb20 or both showed no labeling of cells with Nb20 (FIG. 5C; FIG. 6A). Titration experiments with increasing concentrations of Nb20 showed saturable profile with EC50 of ~10 nM (FIG. 5D,5E). The addition of mGlyR ligand glycine did not affect the binding of Nb20 with mGlyR (FIG. 7). This interaction was specific to mGlyR as the inventors observed no Nb binding with other GPCRs, including related GPR179 and unrelated DIR (FIG. 6B).

Next, the inventors characterized the binding using surface plasmon resonance (SPR). In these experiments the inventors purified the recombinantly expressed extracellular portion of mGlyR (Ecto-mGlyR; SEQ ID NO:12, amino acid residues 1-417 of SEQ ID NO:1 GPR158/mGlyR) tagged with an Fc tag and immobilized on the SPR chip via a mouse anti-human IgG CH2 monoclonal antibody (FIG. 5F). Applying increasing concentrations of Nb20 showed robust binding to the immobilized Ecto-mGlyR with an estimated K_{D}=375 nM (φ²=1.07; K_{ON}=4.19×10⁴ ms⁻¹ and a K_{OFF}=1.57×10⁻² s⁻¹) (FIG. 5G). Together, these data support the development of selective affinity tool for mGlyR-Nb20.

### Example 2: Nb20 Inhibits mGlyR Signaling Via RGS7/Gβ5 Complex

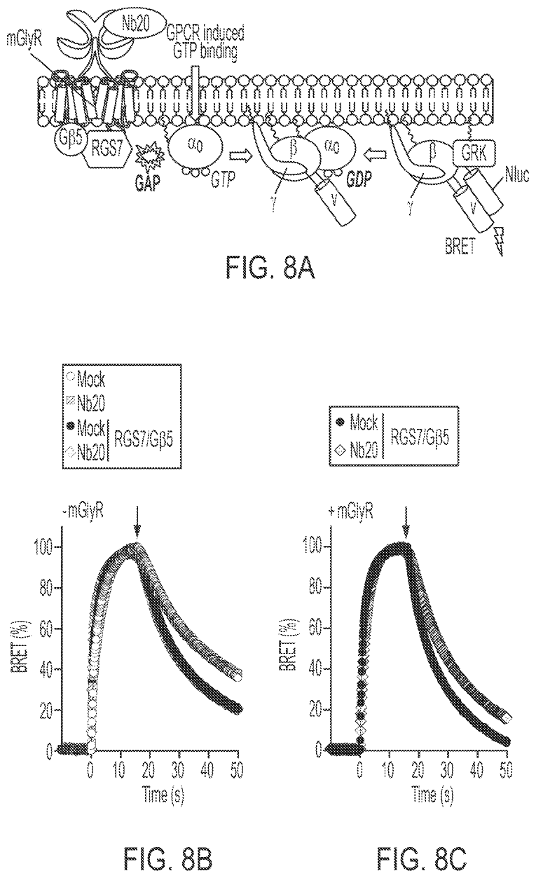

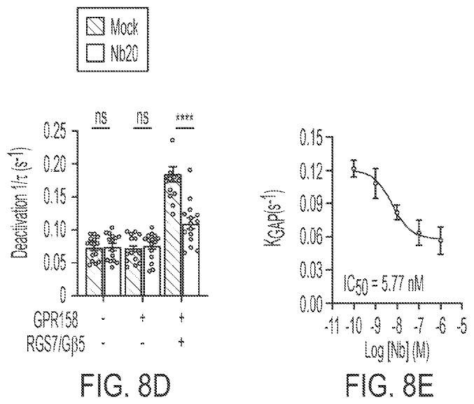

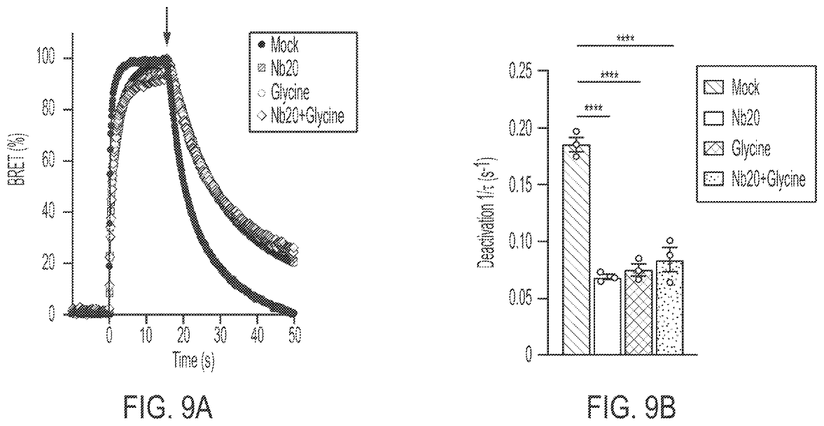

To study the functional consequences of mGlyR interaction with Nb20 the inventors analyzed the ability of mGlyR to modulate the activity of the GTPase Activating Protein (GAP) complex RGS7/Gβ5, through which mGlyR transduces its signals. The inventors used a cell-based assay to monitor GAP activity by following the kinetics of G protein deactivation (FIG. 8A). In this assay, activation of G proteins by GPCR stimulation generates the BRET signal upon interaction of liberated Venus-Gβγ subunits with the masGRK3CT-Nluc reporter. This signal is quenched when Ga deactivation is triggered by GPCR antagonism and recombines with Venus-Gβγ to form inactive heterotrimer. As previously reported (48), the inventors found that the introduction of RGS7/Gβ5 accelerated deactivation of its substrate, Gao (FIG. 8B,8D). Application of Nb20 had no significant effect on either baseline Goo deactivation or RGS7/Gβ5-assisted process (FIG. 8B,8D). However, when mGlyR was co-expressed together with RGS7/Gβ5, Nb20 significantly decelerated Gao deactivation (FIG. 8C,8D) suggesting that it specifically inhibited the GAP activity of RGS7/Gβ5 through mGlyR. Dose-response studies showed that the IC₅₀ of Nb20 on GPR158 is ~6 nM (FIG. 8E). The inventors further explored the effect of Nb20 on mGlyR mediated inhibition of the GAP activity through its endogenous ligand-glycine. The inventors found that both glycine and Nb20 produced very similar inhibition of the GAP activity with no occlusive or additive effects see upon combinatorial application indicating a lack of interplay between these two ligands (FIG. 9A-9B). In summary, these studies show that Nb20 serves as a selective inhibitor of GAP activity mediated by the mGlyR complex.

### Example 3: Nb20 Produces Anti-Depressant Effects in Mice

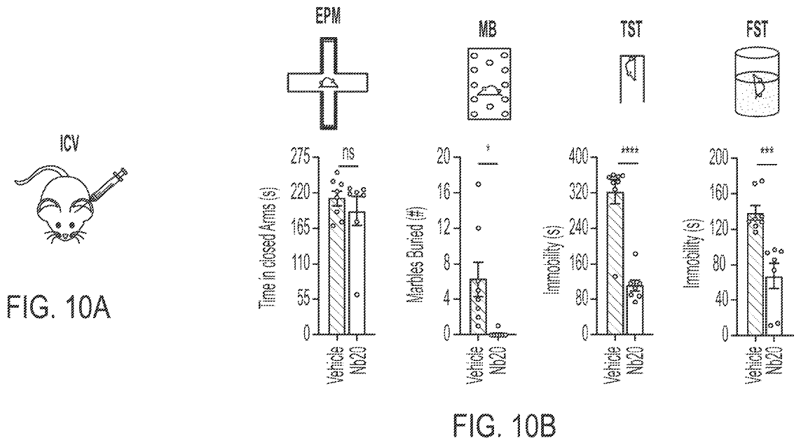

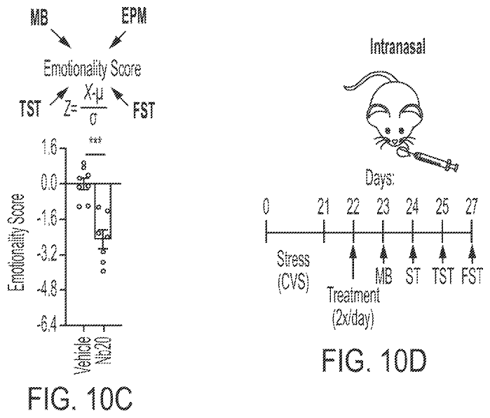

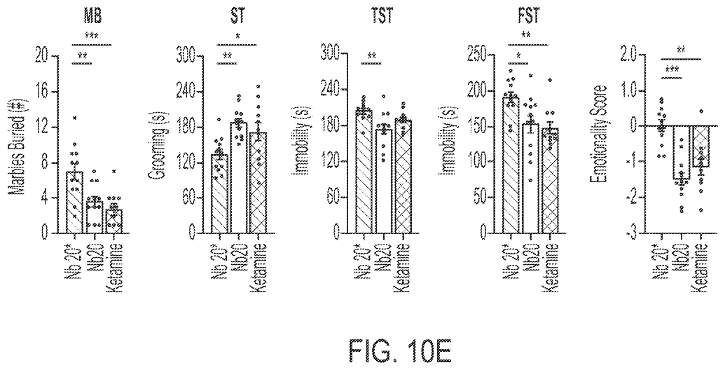

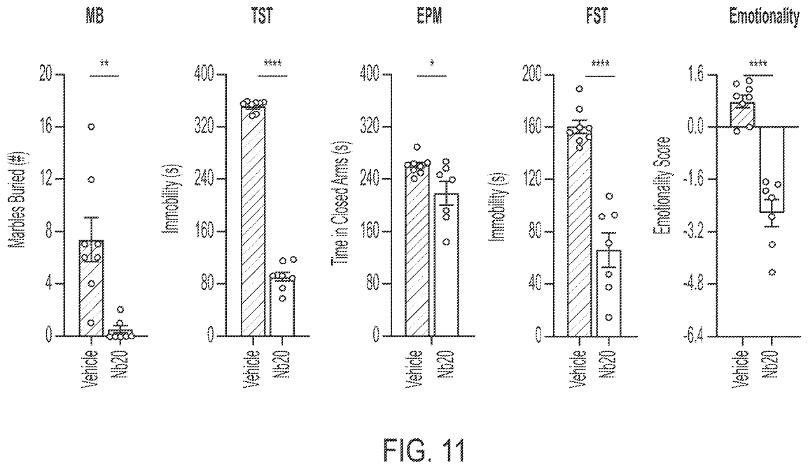

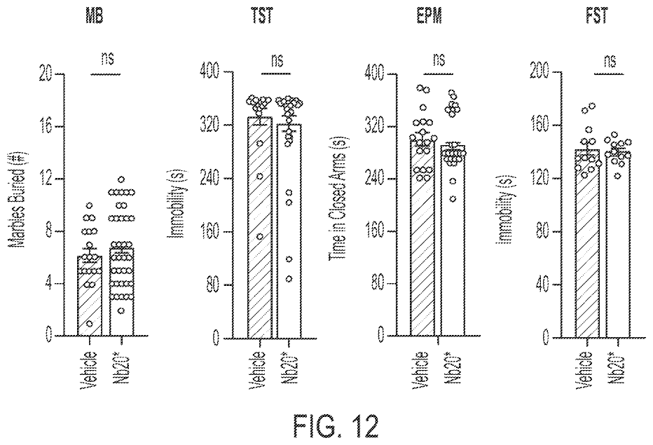

Functional data indicates that Nb20 blocks the ability of mGlyR-RGS7/Gβ5 complex to regulate G protein signaling. Previous studies indicated that genetic deletion of either mGlyR (29) or RGS7 (50) produced substantial antidepressant phenotype in mice and stress resilience. Therefore, the inventors have next tested whether administration of Nb20 in vivo would have similar behavioral effects. Mice were injected, delivering either Nb20 (9.6 μg) or vehicle control into the brain (FIG. 10A) and evaluated in a panel of behavioral tests that assess various aspects of anxiety/depressive-like behaviors 24 hours later (FIG. 10B). The inventors found that mice that received Nb20 buried fewer marbles in the marble burying test, had reduced immobility in the tail suspension and forced swim tests relative to control animals which reflect changes in the depression-related phenotypes but behaved no differently from the control group in the elevated plus maze that broadly measures anxiety-like component (FIG. 10B). Calculating overall emotionality score based on multiple measures confirmed that mice treated with Nb20 displayed prominent antidepressant-like phenotype (FIG. 10C). Remarkably, these behavioral differences between the groups persisted for at least two weeks following the treatment (FIG. 11). To confirm the specificity of the effects, the inventors used mutated Nb20* (SEQ ID NO:13) incapable of binding to mGlyR and found that treatment of mice with mutated Nb20* nanobody did not produce significant effects in any of the paradigms used (FIG. 12). Nb20* differs from Nb20 in CDR-H1 (Nb20* CDR-H1 SEQ ID NO:14) and in CDR-H2 (Nb20* CDR-H2 SEQ ID NO: 15), and has identical CDR-H3 to that of Nb20 (Nb20 CDR-H3 SEQ ID NO:5).

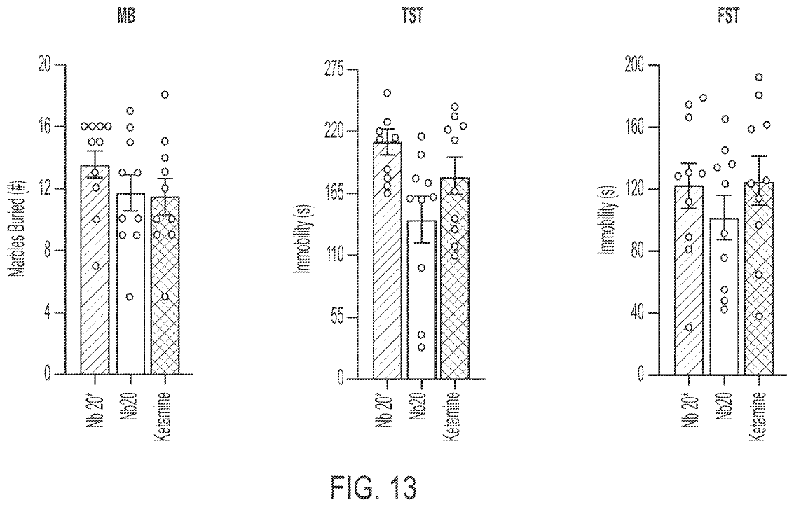

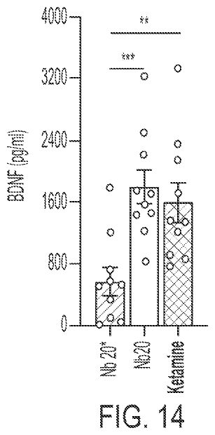

To further explore therapeutic utility of Nb20, the inventors next assessed its effects in a depression model where mice are exposed to a chronic mild stress paradigm. In this study, Nb20 was also administered non-invasively through intranasal delivery, given the translational relevancy of this method for treating depression (53-55). Furthermore, the effects were benchmarked to the effects of rapid anti-depressant treatment with ketamine delivered the same way (FIG. 10D,10E). When mice were tested 24 hours post injection, the inventors observed no statistically significant behavioral changes in ketamine or Nb20 treated mice, with Nb20 treated group showing only a trend relative to mice treated with control Nb20* in tail suspension test (TST; p=0.012; One-way ANOVA) (FIG. 13). Therefore, the inventors repeated the treatments (FIG. 10E) and re-evaluated mice again 24 hours after the second treatment. Remarkably, the inventors found that intranasal delivery of Nb20 also produced a rapid and powerful antidepressant effect in stressed mice (FIG. 10F). The extent of this effect matched the antidepressant effects of ketamine across all behavioral paradigms. Consistent with antidepressant effects, postmortem analysis of the brains revealed marked upregulation of BDNF, a marker for affective states, in PFC of mice treated with Nb20 but not control Nb20* (FIG. 14). Together, these results indicate that Nb20 produces lasting antidepressant effects in mice including models of stress-induced depression.

### Example 4: Inhibition of mGlyR with Nb20 Modulates Physiological Properties of mPFC Neurons

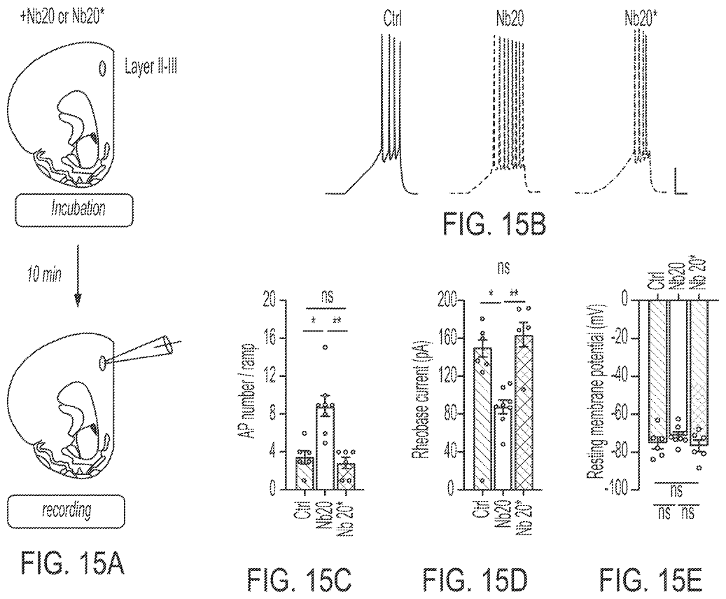

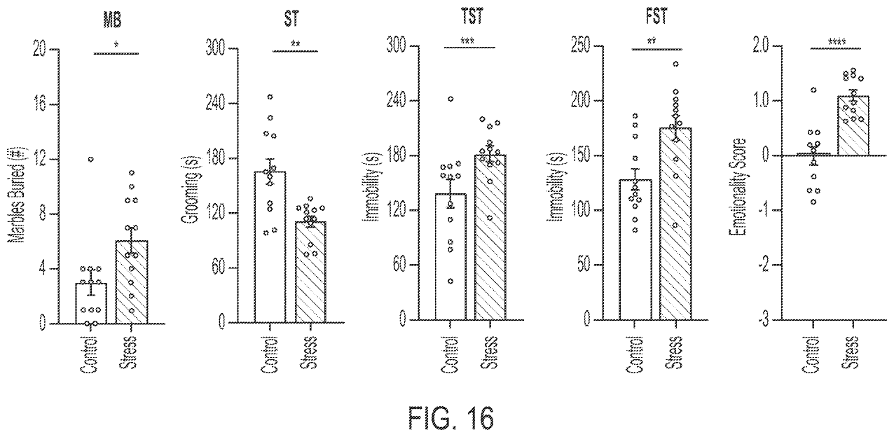

The inventors finally sought to assess the impact of Nb20 on the activity of ex-vivo intact neural networks by examining the intrinsic properties of layer II-III neurons in the prelimbic cortex, where mGlyR is prominently expressed (57). Knockout of mGlyR or its inhibition with endogenous ligand glycine has been shown to increase the excitability of pyramidal neurons in layer II-III (56). Accordingly, the inventors incubated brain slices with Nb20 for 10 minutes prior to recordings and compared excitability of layer II-III neurons with the untreated slices or slices incubated with control Nb20* nanobody deficient in mGlyR binding (FIG. 15A). Pre-incubation with Nb20 significantly increased the number of action potentials fired over the ramp while decreasing the amount of current necessary to elicit the first action potential (FIG. 15B,15C,15D) without changes in the resting membrane potential (FIG. 15E). In contrast, control Nb20* had no effect on neuronal firing, rheobase current or resting membrane potential (FIG. 15B,15C,15D). Together, these results indicate that inhibition of mGlyR with Nb20 specifically increases excitability of layer II-III neurons in prelimbic cortex, an effect previously associated with antidepressant effects.

### Example 5: Materials and Methods

### Animals

All animal experiments were approved by UF Scripps Biomedical Research Institute's Institutional Animal Care and Use Committee (IACUC) in accordance with NIH guidelines. The Gpr158−/− mice were purchased from KOMP (Gpr158tm1(KOMP)Vlcg) and maintained on a C57/β16 background and bred as heterozygous pairs to generate Gpr158−/− and Gpr158+/+ littermates. After weaning male and females were separately group-housed under standard conditions in a pathogen-free facility on a 12:12 light: dark hour cycle with access to food and water ad libitum. For chronic variable stress paradigm, wild-type male and female adult (8-week-old) C57Bl/6J mice (Charles River, Massachusetts) were habituated for one week before experimental manipulation. These mice were single-housed at room temperature (~24° C.) under a 12-hour light/dark cycle (07:00-19:00) with ad libitum access to water and food, except during testing.

cDNA Constructs

Dopamine D2 receptor (cDNA Resource Center: Cat #DRD0200001), RGS7 (GenBank: AY587875), Gβ5 (GenBank: NM_016194), GatoA (cDNA Resource Center: Cat #GNA00A0000) in pcDNA3.1 (+) were purchased from cDNA Resource Center (world wide web.cdna.org). masGRK3ct-Nluc, Venus-156-239-Gβ1, Venus-1-155-Gy2 were synthesized by GenScript in pcDNA3.1+. pCMV5 plasmid encoding GdoA was a gift from H. Itoh (Nara Institute of Science and Technology, Japan). GPR158 ectodomain (aa 1-417) was subcloned in a previously described Fc and 6×His tagged vector (78). Nanobody production were done using pCANTAB vector (phage display) and promising candidates including Nb20 were subcloned in pET28a. To generate control non-binding nanobody (Nb20*), Nb20 cDNA was mutated to replace 30IGNIYI35 (SEQ ID NO:20) sequence in CDR1 with 30GGAGAG35 (SEQ ID NO:21) sequence and 54RTVRWTKYE62 (SEQ ID NO:22) sequence in CDR2 with 54GAVGGAAAG62 (SEQ ID NO:23) sequence using direct mutagenesis by PCR with 2 sets of primers:

CDR1 Forward primer:
(SEQ ID NO: 16)
GCGGCGGTGCTGGCGCTGGCATGGGCTGGTACCGCCAG
CDR1 Reverse primer:
(SEQ ID NO: 17)
CGCCAGCACCGCCGCTTCCAGAGGCTGCACAGGAGAGTC
CDR2 Forward primer:
(SEQ ID NO: 18)
CAGCCGCAGCCGCAGCCGCAGCCGACTATGCAGACTCCGTAAAGGGC
CDR2 Reverse primer:
(SEQ ID NO: 19)
CTGCGGCTGCGGCTGCGGCTGCAGTTGCGACCAGCTCGCG

### Chemical and Drugs

The following chemicals were used. Dopamine hydrochloride (MilliporeSigma Cat #H8502), Haloperidol (MilliporeSigma Cat #H1512), Dulbecco's Phosphate-Buffered Saline (PBS) (Gibco Cat #10010-023) with 0.5 mM MgCl₂ and 0.1% glucose, glycine (National Diagnostics Cat #EC-405), Dulbecco's modified Eagle's medium (Thermo Fisher Scientific Cat #11965-092), Fetal bovine serum (Genesee Scientific Cat #25-550), Sodium pyruvate (Thermo Fisher Scientific Cat #11360-070), MEM non-essential amino acids (Thermo Fisher Scientific Cat #11140-050), Penicillin-streptomycin (Thermo Fisher Scientific Cat #15140-122), Matrigel (Corning Cat #356230), METAFECTENE® PRO (RKP203/RK092820, Biontex Germany), Dulbecco's phosphate-buffered saline (MilliporeSigma Cat #D5652), PEG6000/2M NaCl (Teknova Cat #P4168), Nano-Glo luciferase (N113B Promega), Isopropyl β-D-1-thiogalactopyranoside (IPTG) (Sigma-Aldrich Cat #I6758), Kanamycin (Thermo Fisher Scientific Cat #11815032). The following chemicals were prepared. LEW (50 mM NaH₂PO₄, 300 mM NaCl, pH adjusted to 8.0 using NaOH), 2×YT (16.0 g/l tryptone, 10 g/l yeast extract, 5 g/l sodium chloride, Final pH 6.8±0.2 at 25° C., autoclaved)

### Llama Immunization, Phage Display

One llama was immunized in the strict accordance with good animal practices following the EU animal welfare legislation law with HEK293 cell membrane expressing mGlyR at Eurogentec (Belgium). Blood sample from immunized llama were harvested 87 days after immunization and RNA from leukocytes were extracted using LeukoLOCK Total RNA Isolation system (Life Technologies Cat #AM1923) accordingly to manufacturer instructions. Reverse transcription was performed on extracted RNA and cDNA was amplified by PCR using 8 couples of primers designed to amplify the variable heavy only domains (VHH) of non-conventional IgG2 and IgG3 and introduce NotI and SfiI cleavage site respectively (SfiI cleavage in bold in the following forward primer sequences, and NotI cleavage site in bold in following reverse primer sequences).

### Forward Primers:

VH_11 (SEQ ID NO: 6):
GTCGTCGGCCCAGCCGGCCATGGCCGAGGTGCAGCTGGTGGAGTCTGGG
GGAGG
VH_12 (SEQ ID NO: 7):
GTCGTCGGCCCAGCCGGCCATGGCCGAGGTGCAGCTGCAGGMGTCTGGG
GGAGG
VH_14 (SEQ ID NO: 8):
GTCGTCGGCCCAGCCGGCCATGGCCGAGGTGCAGCTGCAGGCGTCTGG
VH 13 (SEQ ID NO: 9):
GTCGTCGGCCCAGCCGGCCATGGCCGAGGTGCAGCTGCAGGAGTCWGG

### Reverse Primers

VH_sh (SEQ ID NO: 10):
GCTGCTGCGGCCGCGGGGTCTTCGCTGTGGTGCGC
VH_1g (SEQ ID NO: 11):
GCTGCTGCGGCCGCTTGTGGTTTTGGTGTCTTGGG

Amplified cDNA was purified and digested with NotI and SfiI, following by ligation into the phagemid vector pCANTAβ5 and electroporation of competent E. coli TG1 cells. Bacteria were grown in 2×YT broth containing 2% of glucose at 37° C. under agitation until OD_{600 nm} reached 0.5. Subsequently, helper phage KM13 (2×10¹¹ units) were added for 30 min at 37° C. without agitation. Infected bacteria were pelleted by centrifugation at 4000 g for 20 min at 4° C. and pellet resuspended in 2×YT containing 120 μg/ml ampicillin and 50 μg/ml kanamycin. Phage library was grown overnight at 37° C. with agitation. Phages expressing nanobody library were harvested and purified by centrifugation at 8000 g for 15 min 4° C., supernatant were precipitated using 20% of PEG6000/2.5 M NaCl for 1 h at 4° C., followed by centrifugation at 8000 g for 15 min at 4° C. and pellet was resuspended in PBS-glycerol 15% and stored at +4° C.

### Nanobody Identification Via Phage Library Screening

To obtain anti-mGlyR specific nanobody clones, two steps of depletion were first performed. Library containing 10¹² phage units was incubated in a first well of MaxiSorp plate (ThermoFisher Scientific Cat #441653) coated with 100 μg of HEK293 cell membranes mock transfected for 1 h at RT and supernatant was transferred in a second identically coated well for 1 h at RT in order to reduce non-specific binders. A last step of selection was performed using remaining phages incubated on one well coated with 100 μg of cell membranes expressing mGlyR for 2 h at RT. After washes, bound phages were eluted with Tris 50 mM pH=8, 1 mM CaCl₂), Trypsin lug/ml for 15 min at RT. Eluted phages were recovered and amplified with E. coli TG1 cells infected in 2×YT broth containing ampicillin and 2% glucose overnight at 30° C. with agitation. Next day, 2×10⁹ units of helper phage KM13 (AKA VCSM13 Agilent Technologies Cat #2002521) were added to the amplified TG1 cells for 1 h at 37° C. followed by centrifugation at 3,000 g for 10 min at RT. Pellet was resuspended in 2×YT broth containing 100 μg/ml ampicillin and 25 μg/ml kanamycin and incubated overnight at 30° C. with agitation. To separate TG1 cells and amplified phages, overnight culture was centrifuged at 3,000 g for 30 min at 4° C. and phages in the supernatant were precipitated for 30 min on ice using 20% PEG-6000/NaCl 2.5M followed by a centrifugation at 10,000 g for 10 min at 4° C. Pellet was resuspended in PBS with 15% glycerol and stored at −20° C. and the rest was used in the second round of panning selection as described previously. A total of 3 rounds of panning were performed to identify 61 individual clones. Each clone was produced directly from individual TG1 colonies induced by isopropyl-β-26-D-thiogalactopyranoside (IPTG) for flow cytometry screening. Protein concentration in supernatant was determined using UV spectroscopy (Nanodrop).

### Protein Production and Purification

Individual nanobody cDNAs were fused with a c-myc and 8×his tags at the C-terminus, subcloned in pET28a vector using in-fusion HD cloning kit (Takara Bio Cat #102518) and sequenced. Freshly transformed E. coli BL21 DE3 (New England Biolabs Cat #C2527H) with pET28a plasmid encoding nanobody were grown at 37° C., shaking at 220 rpm in Terrific Broth (Kd Medical Inc Cat #501018968) containing 50 μg/ml kanamycin until OD_{280 nm}=[0.6-0.8]. Protein expression was then induced by 1 mM Isopropyl β-D-1-thiogalactopyranoside (IPTG) and bacteria was grown overnight at 28° C., with shaking at 220 rpm. Bacteria were collected, centrifuged and pellet was lysed by sonication in LEW 1× at 4° C. Periplasmic solution were harvested after centrifugation 17000 g, 30 min, 4° C. and purified on Ni-IDA column (Macherey Nagel Cat #745160) according to manufacturer recommendations. Ecto-GPR158-Fc was produced in HEK293FT cells (supernatant) purified (Nickel column) dialyzed/concentrated (Amicon tube 30 kDa). Protein concentration in supernatant was determined using UV spectroscopy (Nanodrop).

### Cryo-EM Sample Preparation and Data Acquisition

The cryo-EM sample was prepared as described previously (32). The protein sample, the purified Nb20-mGlyR-RGS7-Gβ5 complex, was prepared for cryo-electron microscopy (Cryo-EM) imaging. A total of 3.0 μL of the protein sample was applied to glow-discharged 200 mesh gold grids (UltraAufoil R1.2/1.3) inside an FEI Vitrobot Mark IV (Thermo Fischer Scientific). The Vitrobot was maintained at 4° C. with 100% humidity. Prior to blotting, a blot force of 2, blot time of 2 seconds, and a wait time of 20 seconds were applied to remove excess sample. The grids were then plunge-frozen in liquid ethane to preserve their vitrified state.

Cryo-EM imaging of the Nb20-mGlyR-RGS7-Gβ5 protein complex was performed using a 300 kV Titan Krios electron microscope equipped with a Gatan K3 Summit direct electron detection (DED) camera (Gatan, Pleasanton, CA, USA) and a post-column GIF Quantum energy filter operating in counting mode. The microscope was calibrated to a magnification of 105,000, resulting in a nominal pixel size of 0.873 Å. A total of 6689 movies were collected, covering a defocus range of −1.5 to −2.0 μm. The total dose applied was 40 e⁻/Å², achieved by using a dose rate of approximately 12.5 e⁻/s/phys pixel per frame across 40 frames, resulting in a total exposure time of 2.5 seconds.

### Image Processing and 3D Reconstruction

The mGlyR cryo-EM dataset was processed using RELION (79) and cryoSPARC (80). The 6689 movies were initially motion-corrected for beam-induced motion using MotionCor2 (81) within RELION. Motion corrected images were then imported into cryoSPARC (80), where patch Contrast transfer function (CTF) estimation tool was used for CTF estimation.

For particle picking, the TOPAZ algorithm, which employs a convolutional neural networks algorithm implemented in CryoSparc was used (82). A training set of 1000 micrographs were used to generate a trained model, which was subsequently used for particle picking across the entire dataset, resulting in a total of 3,247,619 particles, which were then extracted from the micrographs with a box size of 340. The extracted particles underwent three rounds of reference-free 2D classification to discard poor-quality particles. The protein particles exhibiting favorable 2D class averages were combined and subjected to several rounds of ab-initio and heterogeneous refinement. This iterative refinement process led to the generation of two subsets, one containing 91,859 particles corresponding to the Nb20-mGlyR-RGS complex, and the other consisting of 200, 127 particles corresponding to the mGlyR-Nb20 complex. The final resolution of these subsets was estimated to be approximately 3.47 Å and 3.8 Å, respectively, using the gold standard Fourier shell correlation (GSFSC) method within cryoSPARC (80). EM density visualization was done in UCSF Chimera (83).

### Model Building and Refinement

The models of mGlyR-Nb20 and Nb20-mGlyR-RGS7-Gβ5 were built based on the previously determined structure of mGlyR (PDB ID: 7SHE and 7SHF) using COOT (84). When building the model for mGlyR, the density corresponding to Nb20 was observed, indicating the presence of the bound nanobody. The shape and size of this density were consistent with the bound nanobody. The Nb20 model generated using the AlphaFold method was docked into the cryo-EM map and manually adjusted, built, and refined using COOT (84). Since the resolution of the Nb20 density was relatively low, the density corresponding to bulky residues was used as a guide to accurately build most of the bound Nb20.

The cryo-EM map of Nb20-mGlyR-RGS7-Gβ5 had a final resolution of 3.89 Å. The map exhibited well-resolved density for the transmembrane (TM) domain but relatively low density for RGS7, with the RGS domain missing from the maps. To build the atomic model of the Nb20-mGlyR-RGS7-Gβ5 structure, the mGlyR-Nb20 model generated in this study (from the same dataset) was used as a template. The model was docked into the map using UCSF Chimera and further manually adjusted, built, and refined in COOT.

In the Nb20-mGlyR-RGS7-Gβ5 map, the density for the RGS domain of RGS7 was poorly resolved and fragmented, making it challenging to build a reliable model. Both the mGlyR-Nb20 and Nb20-mGlyR-RGS7-Gβ5 models underwent iterative manual building in COOT, followed by real space refinement in PHENIX (85). Local rotamer fitting and restrained group ADP refinement were also performed. The resulting models were refined against both the unfiltered half maps and sum maps in real space using PHENIX. Structures were visualized and figures were prepared in UCSF Chimera (83), ChimeraX (86-87) and PyMOL(88). The data collection and refinement statistics are listed in Table 6.

TABLE 6
Cryo-EM data collection, refinement and validation statistics

| | Nb20-mGlyR | Nb20-mGlyR-RGS7-Gβ5 |
|---|---|---|
| Data collection and processing | | |
| Voltage (kV) | 300 | 300 |
| Electron exposure (e−/Å²) | 40 | 40 |
| Defocus range (μm) | −1.5 to −2.0 | −1.5 to −2.0 |
| Pixel size (Å) | 0.873 | 0.873 |
| Symmetry imposed | C1 | C1 |
| Initial particle images (no.) | 3,247,619 | 3,247,619 |
| Final particle images (no.) | 263,237 | 225,537 |
| Map resolution (Å) | 3.3 | 3.3 |
| FSC threshold | | |
| Map resolution range (Å) | 2.6 to 7.0 | 2.6 to 7.0 |
| Refinement | | |
| Initial model used (PDB code) | 7SHE | 7SHF |
| Model resolution (Å) | 3.47 | 3.8 |
| FSC threshold | | |
| Model resolution range (Å) | 2.6 to 7.0 | 2.6 to 7.0 |
| Map sharpening B factor (Å²) | −159 | −172 |
| Model composition | | |
| Non-hydrogen atoms | 10931 | 11,009 |
| Protein residues | 1,331 | 1,458 |
| Ligands | 24 | 24 |
| B factors (Å²) | | |
| Protein | 121.72 | 136.77 |
| Ligand | 140.54 | 150.33 |
| R.m.s. deviations | | |
| Bond lengths (Å) | 0.007 | 0.005 |
| Bond angles (°) | 1.15 | 1.06 |
| Validation | | |
| MolProbity score | 2.36 | 2.23 |
| Clashscore | 11.5 | 9.3 |
| Poor rotamers (%) | 0 | 0 |
| Ramachandran plot | | |
| Favored (%) | 82.75 | 83.58 |
| Allowed (%) | 15.67 | 14.58 |
| Disallowed (%) | 1.58 | 1.84 |

### Culture and Transfection of Mammalian Cell Lines

HEK293FT cells were obtained from ThermoFisher and grown in Dulbecco's modified Eagle's medium (DMEM) supplemented with 10% fetal bovine serum (v/v), minimum Eagle's medium non-essential amino acids, 1 mM sodium pyruvate, and antibiotics (100 units/ml penicillin and 100 mg/ml streptomycin) at 37° C. in a humidified incubator containing 5% CO2. Cells were transiently transfected using Metafectene Pro (Biontex, Germany) in 96 well plate following the manufacturer's instructions.

### Cell Based Bioluminescence Resonance Energy Transfer (BRET) Assays

HEK293FT cells were seeded in a white flat-bottom 96 well plate (Greiner Bio-One) at 50,000 cells per well and transiently transfected using Metafectene Pro with the manufacturer instruction. pcDNA3.1 plasmids coding for Dopamine D2R (1), mGlyR (1), RGS7 (1), Gβ5 (1), Gαo (2), Venus-1-155-Gγ2 (1), Venus 156-239-Gβ1 (1) and masGRK3ct-Nluc (1) were used to transfect cells (ratio in parenthesis). An empty vector (pcDNA3.1 (+)) was used in order to normalize the quantity of transfected DNA. 24 h after transfection in DMEM complete medium supplemented of 0.1% of Matrigel (Corning), cells were washed with BRET buffer (Dulbecco's Phosphate-Buffered Saline (PBS) containing 0.5 mM MgCl₂ and 0.1% glucose). Measurements of BRET between Venus-Gβ1γ2 and masGRK3ct-Nluc were performed to monitor the release of free Gβγ dimers after activation of heterotrimers containing Ga subunits in living cells as described before (76). An empty vector (pcDNA3.1(+)) was used in order to normalize the quantity of transfected DNA.

Cells were incubated with 1 μM of Nb20 or with 100 μM of glycine or both when indicated and with the NanoLuc (Nluc) substrate at the manufacturer's instructions. In order to release Gao and start deactivation of Goo, 100 μM of dopamine and 100 μM of haloperidol were injected sequentially and automatically (t=10 s and t=25s) by the plate reader (PHERAstar FSX, BMG Labtech) and dual-luminescence were measured at 475±30 nm and 535±30 nm. The BRET signal was determined by calculating the ratio of the light emitted by the Venus-Gβ_{1γ2} (535 nm with a 30 nm band path width) over the light emitted by the masGRK3ct-Nluc (475 nm with a 30 nm band path width). The average baseline value (basal BRET ratio) recorded prior to agonist stimulation was subtracted from the experimental BRET signal values and the resulting difference (ΔnetBRET ratio) was normalized against the maximal netBRET value recorded upon agonist stimulation. The rate constants (1/τ) of the deactivation phases were obtained by fitting a one phase exponential decay curve to the traces with Graphpad Prism 9.0. The kGAP rate constants were determined by subtracting the basal deactivation rate (kapp) from the deactivation rate measured in the presence of exogenous RGS protein.

### Flow Cytometry

HEK293FT cells were cultured in 6 well plates at the density of 1.10⁶ per well and transfected with 2 μg of cDNA of mGlyR or empty pcDNA3.1+ in control experiments, using Metafectene Pro. 48 h after transfection, cells were mechanically detached pipetting up-down, washed in PBS supplemented with 0.1% BSA, counted and incubated in PBS-0.1% BSA for 1 h at 4° C. under rotation. Nanobody-20 (Nb20) and 10 μl of anti-myc-APC conjugated antibody (R&d Systems #IC3696A) were added and incubated in the dark with rotation, at 4° C. for 1 h. After 3 washes, cells were analyzed in LSR-II BD flow cytometer. Gating strategy to sort individual cells to debris and doublets was used. Sorted cells were measured for fluorescence in respective channels. Negative control conditions were used to set the positive threshold for APC (mock cells incubated with Nb and anti-myc-APC antibody), and for Venus (mock transfected cells). Acquired data were analyzed using FlowJo software (FlowJo).

### Surface Plasmon Resonance (SPR)

Surface plasmon resonance (SPR) measurements were performed on a Biacore X100 instrument at 25° C. using 1×HBS-EP+ (Cytiva) as a running buffer. A mouse anti-human IgG CH2 monoclonal antibody (Cytiva) was immobilized to a density of ~9,500 response units (RU) on a CM5 sensor chip via standard NHS/EDC coupling methods (Cytiva). Subsequently, GPR158-Fc at 10 μg/mL, was captured to ~1,800 RU on the active flow cell. A concentration series with two-fold dilutions (500-31.25 nM) of the nanobody Nb20 were injected using a multi-cycle method. The lowest concentration (31.25 nM) was repeated to confirm regeneration of the sensor chip. Biacore X100 Control Software 2.0.1 (Cytiva) was used to collect data and Biacore X100 Evaluation Software 2.0.1 (Cytiva) to analyze data.

### Brain Slice Preparation and Whole Cell Recordings

Electrophysiological recordings from layer II-III neurons of the prelimbic cortex were performed with mice of either sex aged between 4-12 weeks. Mice were anesthetized with isoflurane and decapitated. The brain was quickly removed and rested for 30 seconds in ice-cold oxygenated solution containing: 93 mM NMDG, 2.5 mM KCl, 1.2 mM NaH₂PO₄, 30 mM NaHCO₃, 20 mM HEPES, 25 mM glucose, 2 mM thiourea, 5 mM Na-ascorbate, 3 mM Na-pyruvate, 0.5 mM CaCl₂), 10 mM MgCl₂, (adjusted to 7.3-7.4 pH with HCl). Coronal slices (300 μm thick) were cut on a vibratome (VT1200S, Leica) mounted on a porous membrane and incubated for 12 min at 34° C. in NMDG. Slices were then transferred to a modified HEPES ACSF containing: 92 mM NaCl, 2.5 mM KCl, 2 mM CaCl₂), 2 mM MgCl₂, 1.2 mM NaH₂PO₄, 30 mM NaHCO₃, 20 mM HEPES, 25 mM glucose, 5 mM Na-ascorbate, 2 mM thiourea, 3 mM Na-pyruvate, (adjusted to 7.3-7.4 pH with NaOH) and allowed to recover for 1 hour at room temperature. For recordings, slices were transferred to a submerged recording chamber where they were continuously perfused at 2 ml/min with oxygenated ACSF containing the following: 126 mM NaCl, 2.5 mM KCl, 2 mM CaCl₂, 2 mM MgCl₂, 18 mM NaHCO₃, 1.2 mM NaH₂PO₄, 10 mM glucose, in presence of the following synaptic blockers: picrotoxin (100 μM), strychnine (1 μM), CNQX (20 μM), APV (50 μM). Pipets (3-5 MΩ) were pulled from P-1000 (Sutter Instruments, CA) and filled with an intracellular solution containing the following: 119 mM K-MeSO4, 12 mM KCl, 1 mM MgCl₂, 0.1 mM CaCl₂, 10 mM HEPES, 1 mM EGTA, 0.4 mM Na-GTP, 2 mM Mg-ATP, (280-300 mOsm, pH 7.3 adjusted with KOH). Slices were incubated with NB-20, NB-20* (1 μM) or ACSF and changes in neuronal excitability were assessed by counting the number of spikes evoked in response to 1-s depolarizing ramp ranging from 0 to 200 pA with a 20-s intertrial interval. The Rheobase current was defined as the minimum current necessary to elicit the first AP. Acquisition was done using Clampex 10.7, MultiClamp 700B amplifier and Digidata 1440A (Molecular Devices, CA). Data were analyzed with Clampfit 10.7.

### ELISA

Prefrontal cortex tissues punches (2 mm) from treated mice were mechanically homogenized in an homogenization buffer containing 20 mM HEPES pH8, 1 mM EDTA, 150 mM NaCl, 2 mM MgCl₂, 1 mM DTT and complete protease inhibitor cocktail (Roche Cat #11836153001) following by centrifugation at 2000 g to remove nuclear debris and immediately frozen (liquid nitrogen). Quantification of BDNF in each sample were determined by diluting supernatant 1:5 in homogenization buffer and using immunoassay ELISA MAX BDNF Deluxe Set (Biolegend Cat #446604) using manufacturer guidelines.

### Mouse Studies

No statistical method was used to predetermine sample size. No data were excluded from the analyses. No specific randomization methods were used. Animals were randomly assigned to experimental groups depending on genotype. Experimenters were blinded to the treatment groups.

Drug treatments. Intracerebroventricular (ICV) administration was performed according to a previously described method (77). Mice were injected with 5 μl of purified and endotoxin free nanobody solution in saline (1.92 mg/ml) of nanobody or 5 μl of vehicle. Behavioral evaluation was conducted 24 hr after treatment. For the intranasal (IN) administration, 24 hr after the last stressor, mice received two treatments on the same day, separated by a 6-hour interval. Each treatment involved the application of 10 μl of purified, endotoxin-free nanobody solution in saline (0.95 mg/ml) or racemic ketamine hydrochloride (20 mg/kg; VetaKet; Patterson Veterinary; Cat #78925834) dissolved in saline, delivering 5 μL to each nostril using a P-10 pipette.

Stress paradigm. The chronic variable stress (CVS) paradigm consists of daily exposure to one of three stressors over 21 consecutive days. All mice are exposed to one stressor per day, and the three stressors are repeated every three days. The times for each stressor vary from day to day to increase unpredictability. The stressors include restraint stress, where mice are placed in ventilated 50 ml conical tubes for 1 hr in the home cage. The next stressor is foot shock, which is performed in shock boxes (6 mice can be simultaneously run). Mice receive 100 mild foot shocks at 0.4 mA for 1 hr at random intervals. The final stressor is 30 min of predator odor exposure. 15 μL of TMT (Fisher Scientific; 501844430) is pipetted onto a cotton tip applicator and placed into a clean empty standard mouse cage. The single-housing is also considered an additional stressor on the mice. Termination criteria were used to determine whether mice should continue in the stress paradigm, including bleeding, excessive weight loss (>20% initial), and hunched or moribund phenotypes. Two mice (one of each sex) died during the stress paradigm. The remaining stressed mice (n=30) were randomly assigned to drug treatment conditions (n=10 per treatment; 5 of each sex). Behavior was run 24 hr after drug treatment in counterbalanced groups, and animals were numbered to maintain blinding in the manually scored tests. 48 hr after the final behavioral test, mice were sacrificed. Marble Burying. Marble burying (MB) was conducted in a standard mouse cage (27×16.5×12.5 cm) with 5 cm corncob bedding and 20 glass marbles overlaid in a 4×5 equidistant arrangement. Background white noise (approximately 70 dB) was used during trials. The mouse was placed in the center of the cage and testing consisted of a 30 min exploration period. The marbles at least half-buried at the end of the trial were counted as buried.

Sucrose Splash Test. The splash test (ST) was performed using a 10% sucrose solution freshly prepared the day of the test (Sigma-Aldrich; S9378) and was sprayed onto the dorsal coat of the mouse (~0.35 mL per mouse), and placed into an empty, inescapable cylindrical PlexiGlas container (121 cm in length and 15 cm in diameter). Mice were habituated to the cylinder for 5 minutes before spraying with sucrose solution and returning to container. The behavior of the mouse was recorded for 5 minutes and later scored manually by blinded observers. The videos were scored for total grooming time. The scores of the observers are averaged to give final values.

Elevated Plus Maze. The elevated plus maze (EPM) was performed using a black, plexiglass elevated plus maze (apparatus with two open and two enclosed arms 33×6 cm with a wall of 25 cm on the closed arm, elevated 60 cm from the floor; Med Associates, St. Albans, VT). Lighting for the maze was set at 200 lux in the center of the plus maze, 270 lux on the open arms, and 120 lux on the closed arms. Background white noise (approximately 70 dB) was used during trials. Mice were placed in the center of the elevated plus maze and left to explore for 5 min in dim light condition. Mice were recorded using Ethovision XT and the time spent in the open and closed arms and the number of entries from the closed to the open arm was calculated.

Tail suspension test. The tails of the mice were wrapped with tape that covered approximately ⅘ of the tail length and then fixed upside down on a hook. The immobility time of each mouse was recorded and tracked over a 6 min period using Ethovision XT. Automated tracking values were validated via manual scoring by a blind observer.

Force Swim Test. The Porsolt Forced Swim Test (FST) was conducted using vertical clear glass cylinder (10 cm in diameter, 25 cm in height) filled with water (25° C.). The mice spent 6 min in the water and immobility was scored from. A mouse was regarded as immobile when floating motionless or making only those movements necessary to keep its head above the water. Automated tracking values were validated via manual scoring by a blind observer.

Analysis of emotionality score. The behavioral paradigm used to calculate the emotionality score were performed in the following order: MB, ST, EPM, TST and FST. To obtain a comprehensive measure for emotionality, the inventors used z-scoring methodology to integrate standard measures of anxiety-like and depressive-like behaviors, as previously described (89). The testing parameters analyzed were as follows: marble burying (number of marbles buried), elevated plus maze (time spent on open arm, number of entries into the open arm), tail suspension test (immobility) and forced swim test (immobility). For each parameter, the z-score for every individual animal was calculated using approach previously described in Sutton et al. (29). Briefly, for each parameter, the z-score of each individual animal was

$$Z = \frac{X - μ}{σ}$$

calculated using the formula where X represents the individual data point, m represents the mean of control group and s rep-resents the standard deviation of the control group. The emotionality score (ES) for each individual subject was first averaged within test, and then

$$E S = \frac{{Z}_{M B} + {Z}_{E P M} + {Z}_{TST} + {Z}_{FST}}{number of tests} .$$

across each test to ensure equal weighting of all tests. The mean emotionality score for each group is an average of the individual within each group for each experiment.

### Data Analysis and Statistics

The functional data shown represent the average±SEM of at least 3 individual experiments each performed in triplicate and expressed as means±standard error means (s.e.m). For the GAP assay, cells with the same transfection conditions were compared on their treatment and two-way ANOVA test was performed. The non-linear regression curve was used to fit on the dose response curve giving the IC₅₀ value. For the flow cytometry binding assays cells transfected with empty vector were used as control and the non-linear regression curve was used to fit on the dose response curve giving the EC₅₀ value. For the SPR, calculation of association (kₒₙ) and dissociation (k_{off}) rate constants was based on a 1:1 Langmuir binding model. All fitting data revealed a Chi² value of <1. The equilibrium dissociation constant (KD) was calculated from k_{off}/kₒₙ. For the mouse experiments, Marble Burying, Splash Test, Tail Suspension, Elevated Plus Maze and Force Swim Test, comparisons were by one-way ANOVA with Dunnett or Kruskal-Wallis post hoc tests. Assumptions of normality and homogeneity of variance were studied using Shapiro-Wilk tests and Levene's tests, respectively. *P<0.05; **P<0.01; ***P<0.001; ****P<0.0001. Mean values with s.e.m. are shown. For the ELISA, one-way ANOVA test was performed to compare control treatment with treated condition. *P<0.05; **P<0.01; ***P<0.001; ****P<0.0001. Mean values with s.e.m. are shown. For the electrophysiology experiments changes was assessed in nonparametric t-test; Wicoxon test. Values of *P<0.05, **P<0.01 and ***P<0.001 ***P<0.0001 were considered to be statistically significant. Calculations, graphs and statistics were generated using GraphPad Prism 9 software (San Diego, CA, USA).

### GPR158 Ectodomain

GPR158 (AKA mGlyR) ectodomain (SEQ ID NO:12) (amino acid residues 1-417 of SEQ ID NO: 1) was subcloned in an Fc and 6×His tagged vector. Ecto-GPR158-Fc was produced in HEK293FT cells (supernatant) purified (Nickel column) dialyzed/concentrated (Amicon tube 30 kDa) before being captured by a mouse anti-human IgG CH2 monoclonal antibody immobilized on a CM5 sensor chip for SPR.

>GPR158-ectodomain:
(SEQ ID NO: 12)
MGAMAYPLLLCLLLAQLGLGAVGASRDPQGRPDSPRERTPKGKPHAQQP
GRASASDSSAPWSRSTDGTILAQKLAEEVPMDVASYLYTGDSHQLKRAN
CSGRYELAGLPGKWPALASAHPSLHRALDTLTHATNFLNVMLQSNKSRE
QNLQDDLDWYQALVWSLLEGEPSISRAAITFSTDSLSAPAPQVFLQATR
EESRILLQDLSSSAPHLANATLETEWFHGLRRKWRPHLHRRGPNQGPRG
LGHSWRRKDGLGGDKSHFKWSPPYLECENGSYKPGWLVTLSSAIYGLQP
NLVPEFRGVMKVDINLQKVDIDQCSSDGWFSGTHKCHLNNSECMPIKGL
GFVLGAYECICKAGFYHPGVLPVNNFRRRGPDQHISGSTKDVSEEAYVC
LPCREGCPFCADDSPCFVQEDKYLR

### Nb20* Construction

>Nb20* (SEQ ID NO:13) (also referred to as Nb20-Ctrl, or Nb20ΔCDR1ΔCDR2) (IMGT definition CDRs bolded and underlined)

MAEVQLQESGGGLVQAGGSLRLSCAASGSGGAGAGMGWYRQTPGPQREL
VATIGAVGGAAAGDYADSVKGRFTISDDDAKNTVYLQMNSLKPEDTAVY
YCNYKDYNAPSDGYWGQGTQVTVSSEPKTPKPQ
IMGT CDR-H1
(SEQ ID NO: 14)
GSGGAGAG
IMGT CDR-H2
(SEQ ID NO: 15)
IGAVGGAAAG
IMGT CDR-H3
(SEQ ID NO: 5)
NYKDYNAPSDGY

### Cloning Strategy:

### Mutagenesis by PCR

CDR1 mutation:
>Forward primer
(SEQ ID NO: 16)
GCGGCGGTGCTGGCGCTGGCATGGGCTGGTACCGCCAG
>Reverse primer
(SEQ ID NO: 17)
CGCCAGCACCGCCGCTTCCAGAGGCTGCACAGGAGAGTC
CDR2 mutation:
>Forward primer
(SEQ ID NO: 18)
CAGCCGCAGCCGCAGCCGCAGCCGACTATGCAGACTCCGTAAAGGGC
>Reverse primer
(SEQ ID NO: 19)
CTGCGGCTGCGGCTGCGGCTGCAGTTGCGACCAGCTCGCG

### Example 6: Structural Basis of mGlyR Regulation by Nb20

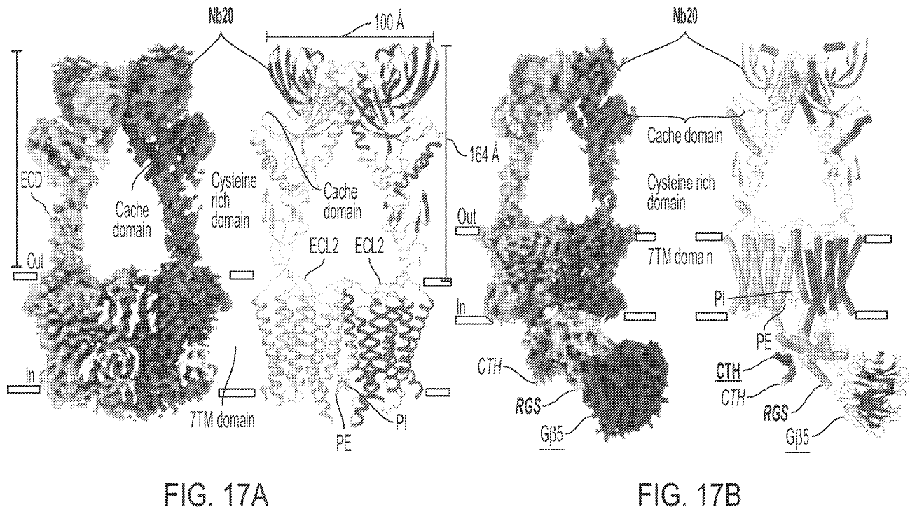

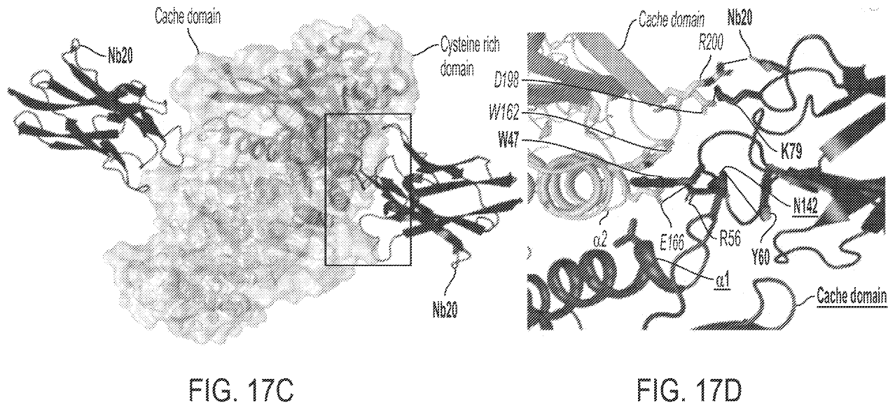

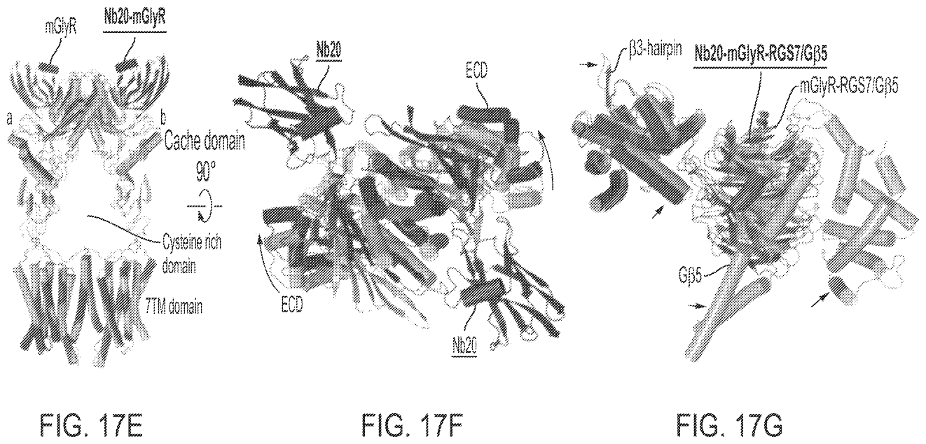

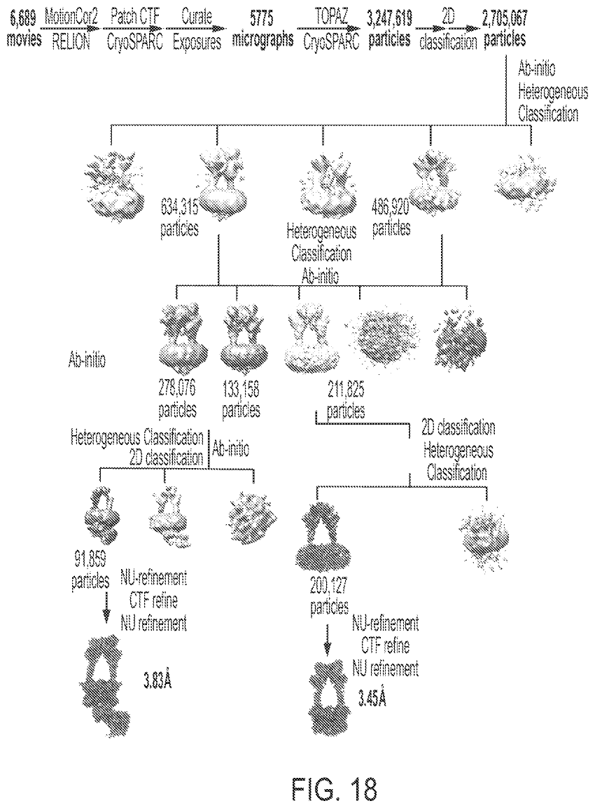

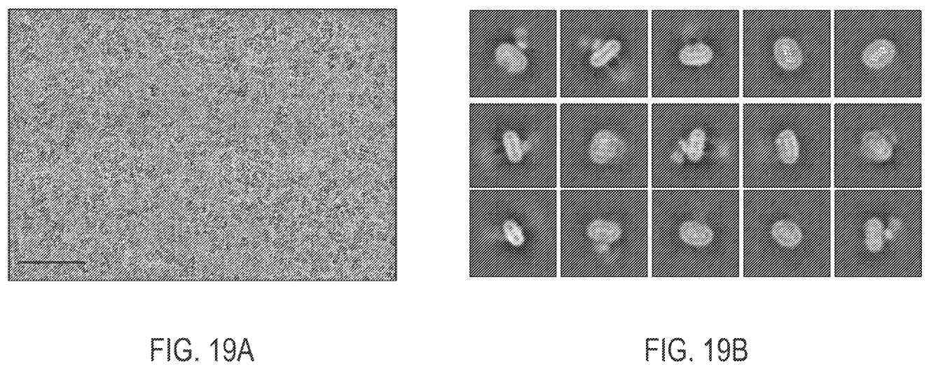

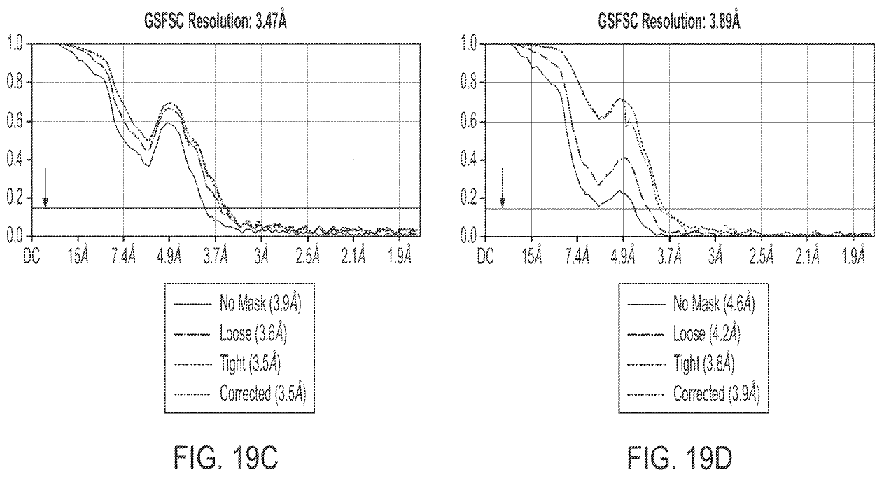

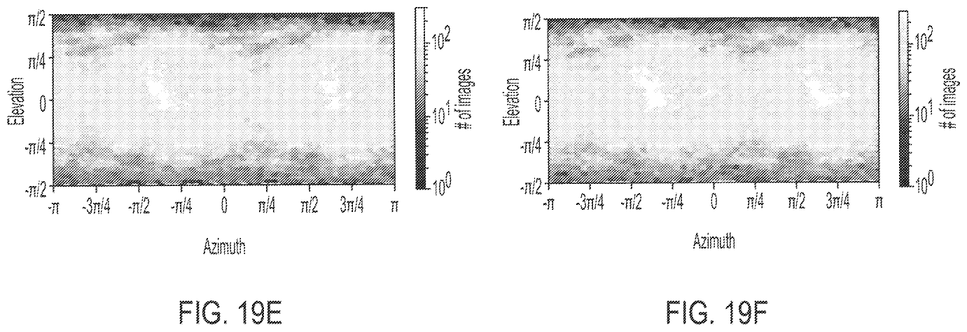

To gain insights into the mechanisms underlying Nb20-mediated modulation of mGlyR, the inventors obtained a high-resolution structure of mGlyR in complex with Nb20 using cryogenic electron microscopy (CryoEM) both with and without RGS7-Gβ5 complex (FIG. 17A,B). Three-dimensional classification (3D) of the particles revealed two dominant 3D classes Nb-20-mGlyR and Nb20-mGlyR-RGS7/Gβ5. Subsequently, these classes were refined to resolutions of 3.45 Å and 3.83 Å, respectively, without applying any symmetry (FIG. 18; FIG. 19). The quality of the obtained maps enabled us to construct the complete models of the mGlyR-Nb20 and mGlyR-RGS complexes, guided by known structures of mGlyR and the nanobodies.

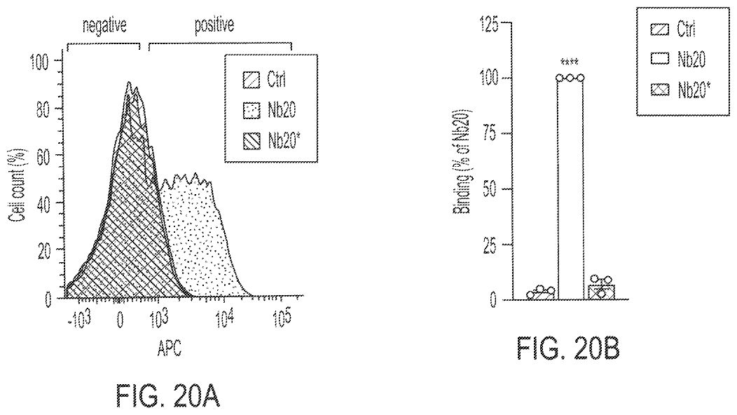

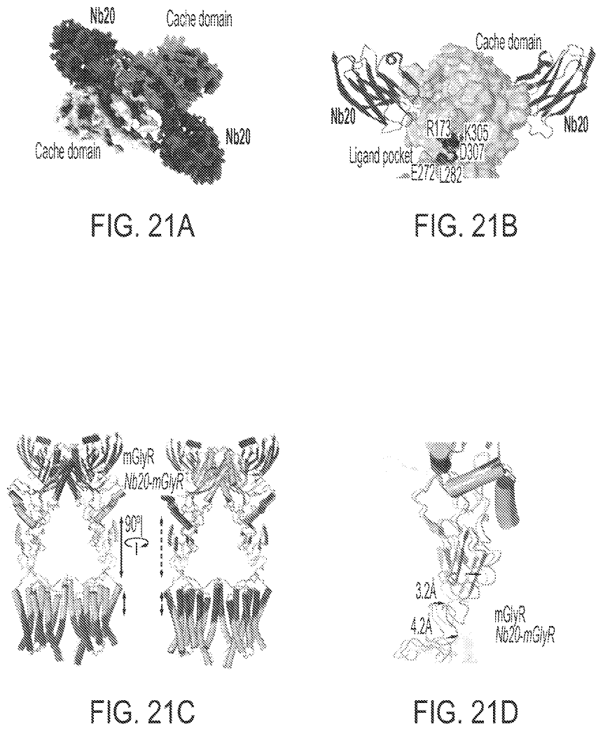

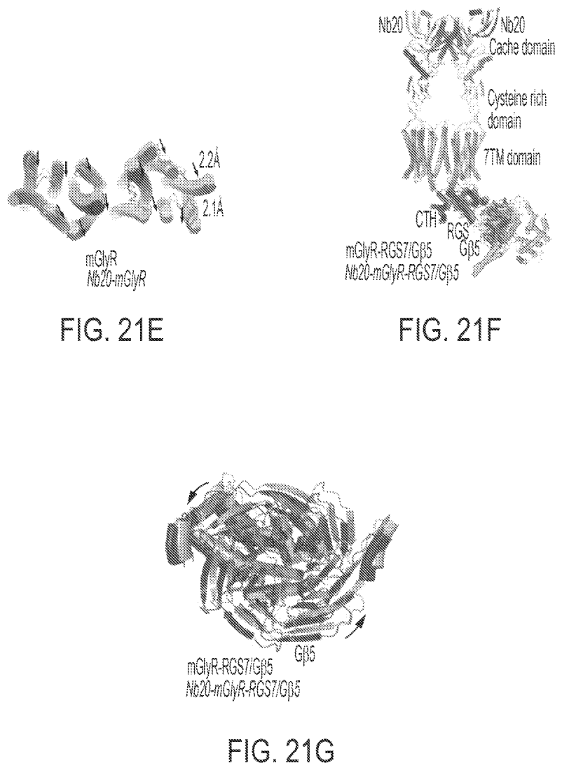

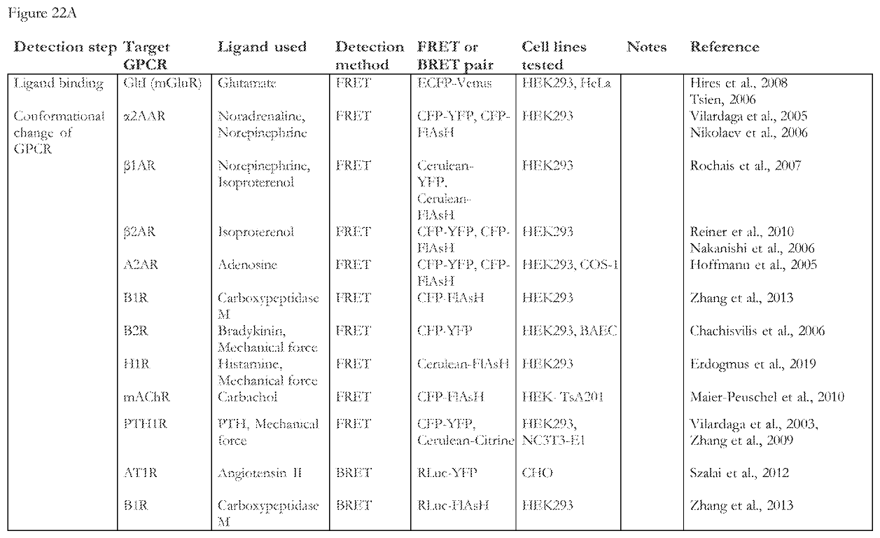

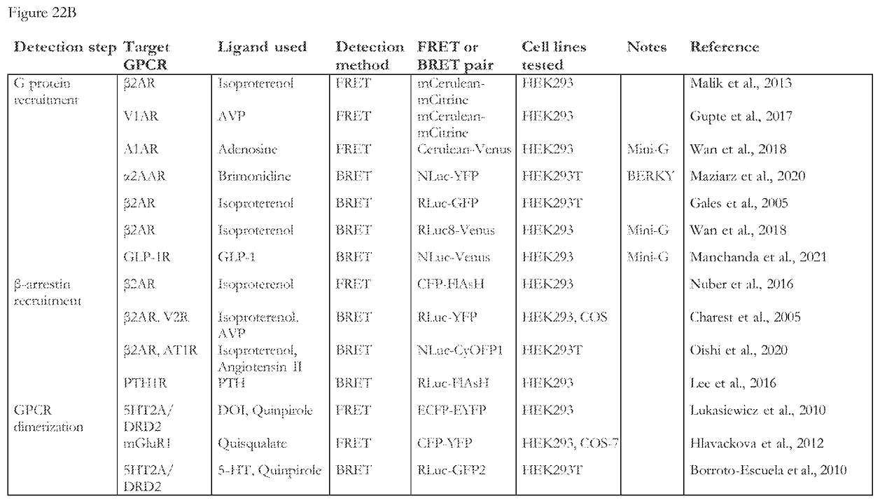

The inventors found that Nb20 was bound to the lateral side of the dimeric interface formed by two ligand binding Cache domains of the mGlyR dimer (FIG. 17C). The binding interface is predominantly mediated by the complementarity-determining regions 1 and 2 (CDR1 and CDR2) of Nb20, establishing extensive contacts with the α2 helix and the loop between the β2 and β3 strands (residues 195-200) of subunit A, as well as a loop between the α1 and α2 helices (residues 140-153) of subunit B of mGlyR (FIG. 17D). The CDR2 of Nb20 interacts with a groove located at the dimeric interface between the two cache domains of the mGlyR receptor. Within the CDR2, the residue W47 forms π-π stacking interactions with W162 from the α2 helix of subunit A of mGlyR. Additionally, polar contacts occur between R56 of Nb20 and E166 of subunit A, as well as between Y60 of Nb20 and N142 of the neighboring subunit of mGlyR (FIG. 17D). Furthermore, Nb20 engages with the receptor through polar interactions involving D77 and K79 from the CDR1 of the nanobody, which interact with R200 and D198 from the loop between residues 195-200 of subunit A of the receptor, respectively. The inventors validated the binding mode of Nb20 by mutating the CDR1 and CDR2 regions involved in the binding which resulted in a complete loss of interaction of mutant Nb20 (Nb20*) with mGlyR (FIG. 20). Notably, the ligand binding pocket is located adjacent to the binding groove of Nb20 (FIG. 21A,B), suggesting that the binding of the Nb20 is likely to affect conformational transitions in mGlyR induced by glycine binding.

To determine whether binding to Nb20 indeed induces conformational changes in mGlyR, the inventors compared Nb20-mGlyR structure to the apo structure of mGlyR alone. Global untethered superimposition of mGlyR-Nb20 on mGlyR-apo revealed conformational changes across various domains, including the Cache, stalk, and transmembrane (TM) domains (FIG. 21C). Notably, the stalk domain exhibited pronounced movements, with deviations of up to 4.2 Å (FIG. 21D). Additionally, the extracellular half of the TM helices displayed an outward shift, while the intracellular half of the TM helices showed an inward movement when compared to the mGlyR-apo structure (FIG. 21E).

To pinpoint the precise conformational changes in the extracellular domain induced by Nb20 binding, the inventors aligned the transmembrane (TM) domains (FIG. 17E). Anchoring the TM that way allowed to observe dramatic conformational changes in the extracellular domain (ECD) upon Nb20 binding to the receptor. Specifically, in the Nb20-bound structure, the ECD translated by as much as 12 Å and rotated by approximately 7° in relation to the mGlyR-apo structure (FIG. 17F). These observations suggest that binding to Nb20 starts the wave of events that eventually remodel the cytoplasmic interface of the receptor involved in interaction with RGS7/Gβ5 and transduction of the signal. To test this model, the inventors further compared the cryoEM structure of Nb20-mGlyR-RGS7/Gβ5 with previously solved mGlyR-RGS7/Gβ5 structure (FIG. 17G; FIG. 21F). In the mGlyR-RGS7/Gβ5 structure, the ECD domain could not be observed. However, the binding of Nb20 stabilized both the ECD and RGS7-Gβ5, allowing to resolve the entire complex. Strikingly, the comparison revealed that the density for the RGS domain, which typically wraps around Gβ5 from below, was entirely absent in the Nb20-bound structure (FIG. 17B; FIG. 21F). Superimposition of Nb20-bound and free structures by the 7TM region showed dramatic changes. The DEP/DEX domain of RGS7 which forms direct contacts with the receptor adopted a distinct conformation with b3-hairpin loop becoming unresolved in the Nb-bound structure (FIG. 17G) suggesting that remodeling in 7TM interface triggers changes in contacting residues of the RGS complex. Furthermore, the Gβ5 in the Nb20-bound structure is translated by approximately 5 Å compared to its position in the free structure (FIG. 17G; FIG. 21G). This distinct conformation of Gβ5 within the complex is not compatible with coupling to the RGS domain which likely becomes flexible and thus unresolved in the structure (FIG. 17G). Together, structural studies suggest a model where binding of Nb20 to the extracellular ligand binding Cache domain of mGlyR induces conformational change in the receptor which remodels its intracellular interaction with the RG7/Gβ5 complex rearranging its catalytic RGS domain to decouple it from regulating Gα proteins.

All patent filings, patents, patent applications, websites, other publications, databases, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### REFERENCES

1. Hernandes, M. S. & Troncone, L. R. P. Glycine as a neurotransmitter in the forebrain: a short review. J Neural Transm 116, 1551-1560 (2009).
2. Legendre, P. The glycinergic inhibitory synapse: CMLS, Cell. Mol. Life Sci. 58, 760-793 (2001).
3. Eulenburg, V., Armsen, W., Betz, H. & Gomeza, J. Glycine transporters: essential regulators of neurotransmission. Trends in Biochemical Sciences 30, 325-333 (2005).
4. Harsing, L. G. & Matyus, P. Mechanisms of glycine release, which build up synaptic and extrasynaptic glycine levels: The role of synaptic and non-synaptic glycine transporters. Brain Research Bulletin 93, 110-119 (2013).
5. Zhu, H. & Gouaux, E. Architecture and assembly mechanism of native glycine receptors. Nature 599, 513-517 (2021).
6. Pérez-Torres, I., Zuniga-Munoz, A. & Guarner-Lans, V. Beneficial Effects of the Amino Acid Glycine. MRMC 17, 15-32 (2016).
7. Han, Y., Zhang, J. & Slaughter, M. M. Partition of Transient and Sustained Inhibitory Glycinergic Input to Retinal Ganglion Cells. J. Neurosci. 17, 3392-3400 (1997).
8. Hou, M., Duan, L. & Slaughter, M. M. Synaptic inhibition by glycine acting at a metabotropic receptor in tiger salamander retina: Metabotropic glycine signal. The Journal of Physiology 586, 2913-2926 (2008).
9. Huang, C.-C. et al. Inhibition of Glycine Transporter-I as a Novel Mechanism for the Treatment of Depression. Biological Psychiatry 74, 734-741 (2013).
10. Li, W. et al. Activation of glycine receptors in the lateral habenula rescues anxiety- and depression-like behaviors associated with alcohol withdrawal and reduces alcohol intake in rats. Neuropharmacology 157, 107688 (2019).
11. Altamura, C., Maes, M., Dai, J. & Meltzer, H. Y. Plasma concentrations of excitatory amino acids, serine, glycine, taurine and histidine in major depression. European Neuropsychopharmacology 5 Suppl, 71-75 (1995).
12. Oja, S. S. & Saransaari, P. Taurine and the Brain. in Taurine 12: A Conditionally Essential Amino Acid (eds. Schaffer, S. W., El Idrissi, A. & Murakami, S.) 325-331 (Springer International Publishing, 2022). doi: 10.1007/978-3-030-93337-1_31.
13. Wettschureck, N. & Offermanns, S. Mammalian G Proteins and Their Cell Type Specific Functions. Physiological Reviews 85, 1159-1204 (2005).
14. Neer, E. J. Heterotrimeric C proteins: Organizers of transmembrane signals. Cell 80, 249-257 (1995).
15. Milligan, G., Canals, M., Pediani, J. D., Ellis, J. & Lopez-Gimenez, J. F. The Role of GPCR Dimerisation/Oligomerisation in Receptor Signalling. in GPCRs: From Deorphanization to Lead Structure Identification (eds. Bourne, H., Horuk, R., Kuhnke, J. & Michel, H.) vol. 2006/2145-162 (Springer Berlin Heidelberg, 2007).
16. Yang, D. et al. G protein-coupled receptors: structure- and function-based drug discovery. Sig Transduct Target Ther 6, 7 (2021).
17. Gurevich, E. V. & Gurevich, V. V. Arrestins: ubiquitous regulators of cellular signaling pathways. Genome Biol 7, 236 (2006).
18. Shukla, A. K., Xiao, K. & Lefkowitz, R. J. Emerging paradigms of β-arrestin-dependent seven transmembrane receptor signaling. Trends in Biochemical Sciences 36, 457-469 (2011).
19. Hermans, E. Biochemical and pharmacological control of the multiplicity of coupling at G-protein-coupled receptors. Pharmacology & Therapeutics 99, 25-44 (2003).
20. Masuho, I. et al. A Global Map of G Protein Signaling Regulation by RGS Proteins. Cell 183, 503-521.e19 (2020).
21. Abramow-Newerly, M., Roy, A. A., Nunn, C. & Chidiac, P. RGS proteins have a signalling complex: Interactions between RGS proteins and GPCRs, effectors, and auxiliary proteins. Cellular Signalling 18, 579-591 (2006).
22. Fajardo-Serrano, A. et al. Association of Rgs7/Gβ5 complexes with girk channels and GABA B receptors in hippocampal CA1 pyramidal neurons: Subcellular Localization OF Rgs7 and Gβ5 in the Hippocampus. Hippocampus 23, 1231-1245 (2013).
23. Celver, J., Sharma, M. & Kovoor, A. D2-Dopamine receptors target regulator of G protein signaling 9-2 to detergent-resistant membrane fractions: D2R targets RGS9-2 to DRM. Journal of Neurochemistry 120, 56-69 (2012).
24. Psigfogeorgou, K. et al. A Unique Role of RGS9-2 in the Striatum as a Positive or Negative Regulator of Opiate Analgesia. Journal of Neuroscience 31, 5617-5624 (2011).
25. Orlandi, C. et al. GPR158/179 regulate G protein signaling by controlling localization and activity of the RGS7 complexes. The Journal of Cell Biology 197, 711-719 (2012).
26. Stockert, J. A. & Devi, L. A. Advancements in therapeutically targeting orphan GPCRs. Front. Pharmacol. 6, (2015).
27. Laschet, C., Dupuis, N. & Hanson, J. The G protein-coupled receptors deorphanization landscape. Biochemical Pharmacology 153, 62-74 (2018).
28. Song, C., Orlandi, C., Sutton, L. P. & Martemyanov, K. A. The signaling proteins GPR158 and RGS7 modulate excitability of L2/3 pyramidal neurons and control A-type potassium channel in the prelimbic cortex. J. Biol. Chem. 294, 13145-13157 (2019).
29. Sutton, L. P. et al. Orphan receptor GPR158 controls stress-induced depression. eLife 7, e33273 (2018).
30. çetereisi, D. et al. Gpr158 Deficiency Impacts Hippocampal CA1 Neuronal Excitability, Dendritic Architecture, and Affects Spatial Learning. Front. Cell. Neurosci. 13, 465 (2019).
31. Khrimian, L. et al. Gpr158 mediates osteocalcin's regulation of cognition. Journal of Experimental Medicine 214, 2859-2873 (2017).
32. Patil, D. N. et al. Cryo-EM structure of human GRP158 receptor coupled to RGS7-Gβ5 signaling complex. 375:86-91 (2022).
33. Jeong, E., Kim, Y., Jeong, J. & Cho, Y. Structure of the class C orphan GPCR GPR158 in complex with RGS7-Gβ5. Nat Commun 12, 6805 (2021).
34. Hong, C., et al. Global trends and regional differences in the burden of anxiety disorders and major depressive disorder attributed to bullying victimisation in 204 countries and territories, 1999-2019: an analysis of the Global Burden of Disease Study. Epidemiol Psychiatr Sci 31, e85 (2022).
35. Zhdanava, M., et al. The Prevalence and National Burden of Treatment-Resistant Depression and Major Depressive Disorder in the United States. J Clin Psychiatry 82 (2021).
36. Marwaha, S., et al. Novel and emerging treatments for major depression. Lancet 401, 141-153 (2023).
37. Dwyer, J. B., et al. Hormonal Treatments for Major Depressive Disorder: State of the Art. Am J Psychiatry 177, 686-705 (2020).
38. Chung, Y. E., et al. Exploration of microbiota targets for major depressive disorder and mood related traits. J Psychiatr Res 111, 74-82 (2019).
39. Lang, U. E. & Borgwardt, S. Molecular mechanisms of depression: perspectives on new treatment strategies. Cell Physiol Biochem 31, 761-777 (2013).
40. Krishnan, V. & Nestler, E. J. The molecular neurobiology of depression. Nature 455, 894-902 (2008)
41. Yang, L., et al. The Effects of Psychological Stress on Depression. Curr Neuropharmacol 13, 494-504 (2015).
42. Belleau, E. L., Treadway, M. T. & Pizzagalli, D. A. The Impact of Stress and Major Depressive Disorder on Hippocampal and Medial Prefrontal Cortex Morphology. Biol Psychiatry 85, 443-453 (2019).
43. Davidson, J. R. Major depressive disorder treatment guidelines in America and Europe. J Clin Psychiatry 71 Suppl E1, e04 (2010).
44. Duman, R. S., Sanacora, G. & Krystal, J. H. Altered Connectivity in Depression: GABA and Glutamate Neurotransmitter Deficits and Reversal by Novel Treatments. Neuron 102, 75-90 (2019).
45. Li, W., et al. Major Depressive Disorder and Kappa Opioid Receptor Antagonists. Transl Perioper Pain Med 1, 4-16 (2016).
46. Gundersen, R. Y., Vaagenes, P., Breivik, T., Fonnum, F. & Opstad, P. K. Glycine—an important neurotransmitter and cytoprotective agent. Acta Anaesthesiol Scand 49, 1108-1116 (2005).
47. Kim, S., Hong, K. B., Kim, S., Suh, H. J. & Jo, K. Creatine and taurine mixtures alleviate depressive-like behaviour in Drosophila melanogaster and mice via regulating Akt and ERK/BDNF pathways. Sci Rep 10, 11370 (2020).
48. Laboute, T., et al. Orphan receptor GPR158 serves as a metabotropic glycine receptor: mGlyR. Science 379, 1352-1358 (2023).
49. Patel, N., Itakura, T., Gonzalez, J. M., Jr., Schwartz, S. G. & Fini, M. E. GPR158, an orphan member of G protein-coupled receptor Family C: glucocorticoid-stimulated expression and novel nuclear role. PLOS One 8, e57843 (2013).
50. Orlandi, C., Sutton, L. P., Muntean, B. S., Song, C. & Martemyanov, K. A. Homeostatic CAMP regulation by the RGS7 complex controls depression-related behaviors. Neuropsychopharmacology (2018).
51. Haubrich, J., et al. A nanobody activating metabotropic glutamate receptor 4 discriminates between homo- and heterodimers. Proc Natl Acad Sci USA 118 (2021).
52. Scholler, P., et al. Allosteric nanobodies uncover a role of hippocampal mGlu2 receptor homodimers in contextual fear consolidation. Nat Commun 8, 1967 (2017).
53. An, D., Wei, C., Wang, J. & Wu, A. Intranasal Ketamine for Depression in Adults: A Systematic Review and Meta-Analysis of Randomized, Double-Blind, Placebo-Controlled Trials. Front Psychol 12, 648691 (2021).
54. Veronesi, M. C., et al. Imaging of intranasal drug delivery to the brain. Am J Nucl Med Mol Imaging 10, 1-31 (2020).
55. Hanson, L. R. & Frey, W. H., 2nd. Intranasal delivery bypasses the blood-brain barrier to target therapeutic agents to the central nervous system and treat neurodegenerative disease. BMC Neurosci 9 Suppl 3, S5 (2008).
56. Rakshit, S. & Molina, J. R. Immunotherapy in patients with autoimmune disease. J Thorac Dis 12, 7032-7038 (2020).
57. Weiner, L. M., Surana, R. & Wang, S. Monoclonal antibodies: versatile platforms for cancer immunotherapy. Nat Rev Immunol 10, 317-327 (2010).
58. Yu, Y. J. & Watts, R. J. Developing therapeutic antibodies for neurodegenerative disease. Neurotherapeutics 10, 459-472 (2013).
59. Slomski, A. Monoclonal Antibody Overcomes Migraine Treatment Failure. Jama 328, 700 (2022).
60. Bateman, R. J., et al. Gantenerumab: an anti-amyloid monoclonal antibody with potential disease-modifying effects in early Alzheimer's disease. Alzheimers Res Ther 14, 178 (2022).
61. Miller, B. J. & Buckley, P. F. Monoclonal antibody immunotherapy in psychiatric disorders. Lancet Psychiatry 4, 13-15 (2017).
62. Zheng, F., et al. Applications of nanobodies in brain diseases. Front Immunol 13, 978513 (2022).
63. Gulati, S., et al. Targeting G protein-coupled receptor signaling at the G protein level with a selective nanobody inhibitor. Nat Commun 9, 1996 (2018).
64. Irannejad, R., et al. Conformational biosensors reveal GPCR signalling from endosomes. Nature 495, 534-538 (2013).
65. Heukers, R., De Groof, T. W. M. & Smit, M. J. Nanobodies detecting and modulating GPCRs outside in and inside out. Curr Opin Cell Biol 57, 115-122 (2019).
66. De Groof, T. W. M., Bobkov, V., Heukers, R. & Smit, M. J. Nanobodies: New avenues for imaging, stabilizing and modulating GPCRs. Mol Cell Endocrinol 484, 15-24 (2019).
67. Sun, S., et al. Nanobody: A Small Antibody with Big Implications for Tumor Therapeutic Strategy. Int J Nanomedicine 16, 2337-2356 (2021).
68. Tsitokana, M. E., Lafon, P. A., Prezeau, L., Pin, J. P. & Rondard, P. Targeting the Brain with Single-Domain Antibodies: Greater Potential Than Stated So Far? Int J Mol Sci 24 (2023).
69. Gao, Y., Zhu, J. & Lu, H. Single domain antibody-based vectors in the delivery of biologics across the blood-brain barrier: a review. Drug Deliv Transl Res 11, 1818-1828 (2021).
70. Rotman, M., et al. Enhanced glutathione PEGylated liposomal brain delivery of an anti-amyloid single domain antibody fragment in a mouse model for Alzheimer's disease. J Control Release 203, 40-50 (2015).
71. Farrington, G. K., et al. A novel platform for engineering blood-brain barrier-crossing bispecific biologics. Faseb J 28, 4764-4778 (2014).
72. Meier, S. R., Sehlin, D. & Syvanen, S. Passive and receptor mediated brain delivery of an anti-GFAP nanobody. Nucl Med Biol 114-115, 121-127 (2022).
73. Haga, K., et al. Nasal delivery of single-domain antibody improves symptoms of SARS-CoV-2 infection in an animal model. PLOS Pathog 17, e1009542 (2021).
74. Gomes, J. R., et al. Delivery of an anti-transthyretin Nanobody to the brain through intranasal administration reveals transthyretin expression and secretion by motor neurons. J Neurochem 145, 393-408 (2018).
75. Dudek, K. A., et al. Molecular adaptations of the blood-brain barrier promote stress resilience vs. depression. Proc Natl Acad Sci USA 117, 3326-3336 (2020).
76. Masuho, I., Martemyanov, K. A. & Lambert, N. A. Monitoring G Protein Activation in Cells with BRET. Methods Mol Biol 1335, 107-113 (2015).
77. Haley, T. J. & McCormick, W. G. Pharmacological effects produced by intracerebral injection of drugs in the conscious mouse. Br J Pharmacol Chemother 12, 12-15 (1957).
78. Condomitti, G., et al. An Input-Specific Orphan Receptor GPR158-HSPG Interaction Organizes Hippocampal Mossy Fiber-CA3 Synapses. Neuron 100, 201-215 e209 (2018).
79. Scheres, S. H. RELION: implementation of a Bayesian approach to cryo-EM structure determination. Journal of structural biology 180, 519-530 (2012).
80. Punjani, A., Rubinstein, J. L., Fleet, D. J. & Brubaker, M. A. cryoSPARC: algorithms for rapid unsupervised cryo-EM structure determination. Nature methods 14, 290 (2017).
81. Zheng, S. Q., et al. MotionCor2: anisotropic correction of beam-induced motion for improved cryo-electron microscopy. Nature methods 14, 331-332 (2017).
82. Bepler, T., Kelley, K., Noble, A. J. & Berger, B. Topaz-Denoise: general deep denoising models for cryoEM and cryoET. Nat Commun 11 (2020).
83. Pettersen, E. F., et al. UCSF Chimera—a visualization system for exploratory research and analysis. Journal of computational chemistry 25, 1605-1612 (2004).
84. Emsley, P. & Cowtan, K. Coot: model-building tools for molecular graphics. Acta Crystallogr D Biol Crystallogr 60, 2126-2132 (2004).
85. Afonine, P. V., et al. Towards automated crystallographic structure refinement with phenix.refine. Acta Crystallogr D 68, 352-367 (2012).
86. Pettersen, E. F., et al. UCSF ChimeraX: Structure visualization for researchers, educators, and developers. Protein Sci 30, 70-82 (2021).
87. Goddard, T. D., et al. UCSF ChimeraX: Meeting modern challenges in visualization and analysis. Protein Sci 27, 14-25 (2018).
88. Afonine, P. V., et al. Towards automated crystallographic structure refinement with phenix.refine. Acta Crystallogr D Biol Crystallogr 68, 352-367 (2012).
89. Guilloux, J. P., Seney, M., Edgar, N. & Sibille, E. Integrated behavioral z-scoring increases the sensitivity and reliability of behavioral phenotyping in mice: relevance to emotionality and sex. J Neurosci Methods 197, 21-31 (2011).

What is claimed is:

1. An isolated monoclonal antibody that competes for binding to human GPR158 with antibody Nb20.

2. The antibody of claim 1 that binds to the same epitope on human GPR158 as antibody Nb20.

3. The antibody of claim 1 or claim 2, comprising three heavy chain CDRs of antibody Nb20, wherein Nb20 is a llama antibody characterized by a heavy chain variable region having an amino acid sequence comprising SEQ ID NO:2.

4. The antibody of claim 3, wherein the three heavy chain CDRs are as defined by IMGT (SEQ ID NOs: 3-5).

5. The antibody of claim 4, wherein the heavy chain variable region comprises an amino acid sequence of SEQ ID NO:2.

6. The antibody of any one of claims 1-4 that is Nb20 or a chimeric, veneered, or humanized form thereof.

7. The antibody of any one of claims 1-4 and 6, wherein the antibody is a humanized antibody.

8. The antibody of claim 6, that is a humanized Nb20 antibody that specifically binds to human GPR158, wherein Nb20 is a llama antibody characterized by a mature heavy chain variable region of SEQ ID NO:2.

9. The humanized antibody of claim 8 comprising a humanized mature heavy chain variable region comprising the three heavy chain CDRs of Nb20.

10. The humanized antibody of claim 9, wherein the CDRs are of a definition selected from the group of Kabat, Chothia, Kabat/Chothia Composite, AbM, Contact, and IMGT.

11. The antibody of any one of claims 1-10 that is an intact antibody.

12. The antibody of any one of claims 1-10 that is a binding fragment.

13. The antibody of claim 12, wherein the binding fragment is a single-chain antibody, Fab, or F(ab′)₂ fragment.

14. The antibody of any one of claims 1-10 that is a Fab fragment, or single chain Fv.

15. The antibody of any one of claims 1-10 that is a nanobody.

16. The antibody of any one of the preceding claims, wherein the isotype is human IgG1.

17. The humanized antibody of any one of claims 7-11 and 16 wherein the mature heavy chain variable region is fused to a heavy chain constant region.

18. The humanized antibody of claim 17, wherein the heavy chain constant region is a mutant form of a natural human heavy chain constant region which has reduced binding to a Fcγ receptor relative to the natural human heavy chain constant region.

19. The humanized antibody of claim 17 or claim 18, wherein the heavy chain constant region is of IgG1 isotype.

20. The antibody of any one of the preceding claims having at least one mutation in the constant region.

21. The antibody of claim 20, wherein the mutation reduces complement fixation or activation by the constant region.

22. The antibody of claim 21 having a mutation at one or more of positions 241, 264, 265, 270, 296, 297, 318, 320, 322, 329 and 331 by EU numbering.

23. The antibody of claim 22 having alanine at positions 318, 320 and 322.

24. The antibody of any one of claims 1-18 wherein the antibody is of human IgG2, IgG3, or IgG4 isotype.

25. The antibody of any one of claims 1-24, wherein the antibody is at least 95% w/w pure.

26. The antibody of any preceding claim, wherein the antibody is conjugated to a therapeutic, cytotoxic, cytostatic, neurotrophic, or neuroprotective agent.

27. A pharmaceutical composition comprising an antibody as defined in any of claims 1-26 and a pharmaceutically-acceptable carrier.

28. A nucleic acid encoding the heavy chain of an antibody as described in any one of claims 1-26.

29. A recombinant expression vector comprising a nucleic acid of claim 28.

30. A host cell transformed with the recombinant expression vector of claim 29.

31. A method of humanizing a nanobody, the method comprising:
(a) selecting one or more acceptor humanized nanobody scaffolds;
(b) identifying amino acid residues of the nanobody to be retained;
(c) synthesizing a nucleic acid encoding a humanized heavy chain comprising CDRs of the nanobody heavy chain; and
(d) expressing the nucleic acids in a host cell to produce a humanized nanobody;
wherein the nanobody is Nb20, wherein Nb20 is characterized by a mature heavy chain variable region of SEQ ID NO:2.

32. A method of producing a humanized, chimeric, or veneered antibody, the method comprising:
(a) culturing cells transformed with a nucleic acid encoding the heavy chain of the antibody, so that the cells secrete the antibody; and
(b) purifying the antibody from cell culture media;
wherein the antibody is a humanized, chimeric, or veneered form of an antibody characterized by a mature heavy chain variable region of SEQ ID NO:2.

33. A method of producing a cell line producing a humanized, chimeric, or veneered antibody, the method comprising:
(a) introducing a vector encoding a heavy chain of an antibody and a selectable marker into cells;
(b) propagating the cells under conditions to select for cells having increased copy number of the vector;
(c) isolating single cells from the selected cells; and
(d) banking cells cloned from a single cell selected based on yield of antibody;
wherein the antibody is a humanized, chimeric, or veneered form of an antibody characterized by a mature heavy chain variable region of SEQ ID NO:2.

34. The method of claim 33 further comprising propagating the cells under selective conditions and screening for cell lines naturally expressing and secreting at least 100 mg/L/10⁶ cells/24 h.

35. A method of treating or effecting prophylaxis of an affective disorder, a mood disorder, or a brain disorder in a subject, comprising administering to the subject an effective regime of an antibody as defined by any one of claims 1-26 and thereby treating or effecting prophylaxis of the affective disorder, mood disorder, or brain disorder in the subject.

36. The method of claim 35, wherein the affective disorder, mood disorder, or brain disorder is depression, disruptive mood dysregulation disorder, major depressive disorder (MDD), dysthymia, stress induced depression, a generalized mood disorder, chronic stress disorder, anhedonia, or an anxiety disorder.

37. A method of detecting of GPR158 in a biological sample from a subject, comprising contacting the biological sample with an effective amount of the antibody of any one of claims 1-25.

38. The method of claim 37, further comprising detecting the binding of antibody to GPR158.

39. The method of claim 37 or claim 38, further comprising comparing binding of the antibody to the biological sample with binding of the antibody to a control sample.
